SEQUENCE LISTING (139 sequences)

SEQ ID NO: 1 - DNA, 110 bp, synthetic construct
  source 1..110: mol_type = other DNA
gattcgtcagtagggttgtaaaggtttttcttttcctgagaaaacaaccttttgttttctcaggttttgctttttggcctttccctagctttaaaaaaaaaaaagcaaaa

SEQ ID NO: 2 - DNA, 59 bp, synthetic construct
  source 1..59: mol_type = other DNA
aaaggtttttcttttcctgagaaatttctcaggttttgctttttaaaaaaaaagcaaaa

SEQ ID NO: 3 - DNA, 50 bp, synthetic construct
  source 1..50: mol_type = other DNA
ctcaccgaggcagttccataggatggcaagatcctggtattggtctgcga

SEQ ID NO: 4 - DNA, 64 bp, synthetic construct
  source 1..64: mol_type = other DNA
gacgctggtggctggcactcctggtttccaggacggggttcaagtccctgcggtgtctttgctt

SEQ ID NO: 5 - DNA, 174 bp, synthetic construct
  source 1..174: mol_type = other DNA
gattcgtcagtagggttgtaaaggtttttcttttcctgagaaaacaaccttttgttttctcaggttttgctttttggcctttccctagctttaaaaaaaaaaaagcaaaagacgctggtggctggcactcctggtttccaggacggggttcaagtccctgcggtgtctttgctt

SEQ ID NO: 6 - DNA, 123 bp, synthetic construct
  source 1..123: mol_type = other DNA
aaaggtttttcttttcctgagaaatttctcaggttttgctttttaaaaaaaaagcaaaagacgctggtggctggcactcctggtttccaggacggggttcaagtccctgcggtgtctttgctt

SEQ ID NO: 7 - RNA, 22 nt, synthetic construct
  source 1..22: mol_type = other RNA
gttttagagctatgctgttttg

SEQ ID NO: 8 - RNA, 12 nt, synthetic construct
  source 1..12: mol_type = other RNA
gttttagagcta

SEQ ID NO: 9 - RNA, 70 nt, synthetic construct
  source 1..70: mol_type = other RNA
aaacagcatagcaagttaaaataaggctagtccgttatcaacttgaaaaagtggcaccgagtcggtgctt

SEQ ID NO: 10 - RNA, 42 nt, synthetic construct
  source 1..42: mol_type = other RNA
gttttagagctagaaatagcaagttaaaataaggctagtccg

SEQ ID NO: 11 - RNA, 78 nt, synthetic construct
  source 1..78: mol_type = other RNA
gttttagagctagaaatagcaagttaaaataaggctagtccgttatcaacttgaaaaagtggcaccgagtcggtgctt

SEQ ID NO: 12 - RNA, 20 nt, synthetic construct
  source 1..20: mol_type = other RNA
aatttctactaagtgtagat

SEQ ID NO: 13 - RNA, 19 nt, synthetic construct
  source 1..19: mol_type = other RNA
aatttctactcttgtagat

SEQ ID NO: 14 - RNA, 19 nt, synthetic construct
  source 1..19: mol_type = other RNA
aatttctactgttgtagat

SEQ ID NO: 15 - RNA, 19 nt, synthetic construct
  source 1..19: mol_type = other RNA
aatttctactattgtagat

SEQ ID NO: 16 - RNA, 20 nt, synthetic construct
  source 1..20: mol_type = other RNA
aatttctactgtttgtagat

SEQ ID NO: 17 - RNA, 19 nt, synthetic construct
  source 1..19: mol_type = other RNA
aatttctactgttgtagat

SEQ ID NO: 18 - RNA, 19 nt, synthetic construct
  source 1..19: mol_type = other RNA
aatttctactattgtagat

SEQ ID NO: 19 - RNA, 36 nt, synthetic construct
  source 1..36: mol_type = other RNA
gtaagagactacctctatatgaaagaggactaaaac

SEQ ID NO: 20 - RNA, 36 nt, synthetic construct
  source 1..36: mol_type = other RNA
gatatagaccaccccaatatcgaaggggactaaaac

SEQ ID NO: 21 - RNA, 35 nt, synthetic construct
  source 1..35: mol_type = other RNA
atttagaccaccccaaaaatgaaggggactaaaac

SEQ ID NO: 22 - RNA, 30 nt, synthetic construct
  source 1..30: mol_type = other RNA
gaccaccccaaaaatgaaggggactaaaac

SEQ ID NO: 23 - RNA, 36 nt, synthetic construct
  source 1..36: mol_type = other RNA
gatttagactaccccaaaaacgaaggggactaaaac

SEQ ID NO: 24 - RNA, 32 nt, synthetic construct
  source 1..32: mol_type = other RNA
gtcacaactcccatgtaggcggagactgcaac

SEQ ID NO: 25 - RNA, 37 nt, synthetic construct
  source 1..37: mol_type = other RNA
ggatttagagtaccccaaaaatgaaggggactaaaac

SEQ ID NO: 26 - protein, 1368 aa, Streptococcus pyogenes [sequence omitted]

SEQ ID NO: 27 - protein, 1087 aa, Geobacillus stearothermophilus
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 28 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 29 - protein, 1121 aa, Streptococcus thermophilus
MSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTLYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINDKGKEVPCNPFLKYKEEHGYIRKYSKKGNGPEIKSLKYYDSKLGNHIDITPKDSNNKVVLQSVSPWRADVYFNKTTGKYEILGLKYADLQFDKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 30 - protein, 1082 aa, Neisseria meningitidis
MAAFKPNPINYILGLDIGIASVGWAMVEIDEEENPIRLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVANNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSSELQDEIGTAFSLFKTDEDITGRLKDRVQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLVRLNEKGYVEIDHALPFSRTWDDSFNNKVLVLGSENQNKGNQTPYEYFNGKDNSREWQEFKARVETSRFPRSKKQRILLQKFDEDGFKECNLNDTRYVNRFLCQFVADHILLTGKGKRRVFASNGQITNLLRGFWGLRKVRAENDRHHALDAVVVACSTVAMQQKITRFVRYKEMNAFDGKTIDKETGKVLHQKTHFPQPWEFFAQEVMIRVFGKPDGKPEFEEADTPEKLRTLLAEKLSSRPEAVHEYVTPLFVSRAPNRKMSGAHKDTLRSAKRFVKHNEKISVKRVWLTEIKLADLENMVNYKNGREIELYEALKARLEAYGGNAKQAFDPKDNPFYKKGGQLVKAVRVEKTQESGVLLNKKNAYTIADNGDMVRVDVFCKVDKKGKNQYFIVPIYAWQVAENILPDIDCKGYRIDDSYTFCFSLHKYDLIAFQKDEKSKVEFAYYINCDSSNGRFYLAWHDKGSKEQQFRISTQNLVLIQKYQVNELGKEIRPCRLKKRPPVR

SEQ ID NO: 31 - protein, 1081 aa, Geobacillus thermodenitrificans
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRP

SEQ ID NO: 32 - protein, 1084 aa, Corynebacterium diphtheriae
MKYHVGIDVGTFSVGLAAIEVDDAGMPIKTLSLVSHIHDSGLDPDEIKSAVTRLASSGIARRTRRLYRRKRRRLQQLDKFIQRQGWPVIELEDYSDPLYPWKVRAELAASYIADEKERGEKLSVALRHIARHRGWRNPYAKVSSLYLPDGPSDAFKAIREEIKRASGQPVPETATVGQMVTLCELGTLKLRGEGGVLSARLQQSDYAREIQEICRMQEIGQELYRKIIDVVFAAESPKGSASSRVGKDPLQPGKNRALKASDAFQRYRIAALIGNLRVRVDGEKRILSVEEKNLVFDHLVNLTPKKEPEWVTIAEILGIDRGQLIGTATMTDDGERAGARPPTHDTNRSIVNSRIAPLVDWWKTASALEQHAMVKALSNAEVDDFDSPEGAKVQAFFADLDDDVHAKLDSLHLPVGRAAYSEDTLVRLTRRMLSDGVDLYTARLQEFGIEPSWTPPTPRIGEPVGNPAVDRVLKTVSRWLESATKTWGAPERVIIEHVREGFVTEKRAREMDGDMRRRAARNAKLFQEMQEKLNVQGKPSRADLWRYQSVQRQNCQCAYCGSPITFSNSEMDHIVPRAGQGSTNTRENLVAVCHRCNQSKGNTPFAIWAKNTSIEGVSVKEAVERTRHWVTDTGMRSTDFKKFTKAVVERFQRATMDEEIDARSMESVAWMANELRSRVAQHFASHGTTVRVYRGSLTAEARRASGISGKLKFFDGVGKSRLDRRHHAIDAAVIAFTSDYVAETLAVRSNLKQSQAHRQEAPQWREFTGKDAEHRAAWRVWCQKMEKLSALLTEDLRDDRVVVMSNVRLRLGNGSAHKETIGKLSKVKLSSQLSVSDIDKASSEALWCALTREPGFDPKEGLPANPERHIRVNGTHVYAGDNIGLFPVSAGSIALRGGYAELGSSFHHARVYKITSGKKPAFAMLRVYTIDLLPYRNQDLFSVELKPQTMSMRQAEKKLRDALATGNAEYLGWLVVDDELVVDTSKIATDQVKAVEAELGTIRRWRVDGFFSPSKLRLRPLQMSKEGIKKESAPELSKIIDRPGWLPAVNKLFSDGNVTVVRRDSLGRVRLESTAHLPVTWKVQ

SEQ ID NO: 33 - protein, 983 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFK

SEQ ID NO: 34 - protein, 1065 aa, Clostridium perfringens
MNKDINYALGLDIGITSVGWAVINLDLNRIEDLGVRIFNAAENLKDGSSLALPRRLARGRRRLLRRKAYRVERVRKLILKNNILTKEELEGLFKNKEVIDVWEARVKGLDYKLSREEWAKILINFCKRRGFKSNRKNEAKDKEAGQILSSISYNIEKMKETNSRTIGEYIYNEVKNSEDSYAPLRNKFGEYKMCVSRDVIREEIHTLFEKQRELGNNFASDEIEDKYFEIFNSQRPFSNFEDLEKLVGFCTFERKKHKRAPKHCISSEEFTLYEGINKLSIIKDGEKRKLSEEERTLVVNEAFNKKEIKYTTLRKLLGLSEEELFSTLTYSIDKDISKTENTKFVSLKGYHEIKRAIEKGVSKKAWQDIENNRNLLNDIAYVLTLGKTDDEIEKQLRLRNVPEELFESLLDMSFNKFNNLSIYALEKILPFMKEGYQYNEACEKAGYNFKAIYEGVRTKKLPVIEIDEIVNPVVNRALAQTRKVINSVIDKYGSPVRINIELARDLAKNFKDRKLIEKEQKENRANLDKIRENLRELMDKEPTVVEVLKYRLWEQQRGECAYTQSQIPLERLFSPGYCEIDHIIPFSRSFDDSLSNKILVLGSENQRKGNRTPYEYFGEDSERWNSFEIWVKGSYLNYKKKANLLKKKFSEEEQRDWKARNLQDTKYICRYISNFVNNRLEFKESENKQKVITVNGRATAYLRSRWGLNKVREDGDKHHALDAAVVGVTTQGMVQKISKYSKAHELYTVRKNDEFVDVETGECVDVDEYKEIRKDILPRPWRGFSEELKLRLSDNPLEELKKSPIETYNEEFIKENVKPIFVSRVPFRKISGKLFKETIYSERAFKDGYFVSKKSLVDLKRGDLDNFYNYECDKKLYDAISERMKEFNYNGKKAFESPFRKPTKSGKEGPVVRSVKIKSAVPFKDGIPMNEGLVAKEGMVRIDIYEKEGKYFVVPVYRYQLAKGIVPKRAAIAKKDELKWPIMDQTYNFKFSIYKNDLIEVKYDKKGVYFGYYDGFDRNSASLTIEKHDNSERYRSIGLKVGVLEINKYEVDVLGNYHKVKLGGN

SEQ ID NO: 35 - protein, 1060 aa, Kingella kingae
MAAKSLNYILGLDLGIASCGWAVVEMDEQENPLRLIDVGVRTFEEAENPKDGSSLAETRRLARAQRRLISRRANRMNKLCRLLKQENVLQANDYDEKGLIIGLPNQAWELRAQGLDRKLEPKEWAAVLIHLVKHRGYLSQRKNESQTADKELGALLSGMNHNHQLLQSQSNQYRTPAELAIKQFATQDGHIRNQRGAYTHTFNRLDLQNELNQLFDAQTRLGNPHVSGSLKTAINDLLMNQKAALSGDAILEMLGRCTFEPSEYKAAKNTYSAERFVWLGKLTNLRIQDNGLESALSREQRELLLNEPYNKTKLTYQQVRKLLRLPETAFFKGLRYGKDNAESATFMEMKSYHAIRKSLENKSLKSEWEKLKTQPKLLDLIGTAFSIYKTDDEIAGCLKNQLPENVIIALQGQLNFSNFMELSLKALEKLLPLMEQGLRYDEACTQIYGNRHEKIADKTEKFLPNIPADKLRNPVVLRTLTQARKVINAIIRRYGSPARVHIETGREVGKSFSDRDKLKKQQEENQEQRRRAIDEWKQFFPNAAHEPKAVDILKLRLYQLQQGKCLYSGLPIDVRRLPEKGYVEIDHALPFSRTWDDSFNNKILVLASENQNKGNQTPYEYLRGADNSESWRLFVENVQNCQFSPNKKQKIMAKQLDEKGFLERNLNDTRYVSRFLCQFISEHLHLTGKGKKRVFASNGQITALLRGRWGLSKNREENDRHHAVDAVVVACSTVAMQQKITQFVRYQKGNVFTGERIDKETGEVISIHFPTPWEHFREEVMIRVFDDNPLRTLPEKLPNRPEALHEYVMPLFVSRMPTRKMTGQGHKETIKSAKRLDEKISVLRVPLTQLKLNDLENMVNREREPALYEALKARLEQFKNDPAKAFAEPFFKQGGQQVKAVRVEIVQKSGMLLRKINGVADNGSMVRVDVFEKSGKFYLVPIYAWQVAEGILPNYAVVAHKDESDWILMDESYHFKFALYKNDLLEVQTKKGGIMGYFASLDRATGAITIREHDRDKKKCKDGEHRSIGVKMASSFKKLQVDELGKNIHETRSRKRPNIG

SEQ ID NO: 36 - protein, 1049 aa, Tistrella mobilis
MINERLTFGIDLGIGSCGWAVLRHADAAGEPGVIEGLGSWCFDVPETSKERIPTNQVRRSNRLLRRVIRRRRNRMAEIRRLFHAQGLLPSADEDALKRPRLDPWELRARGLDQCLSAEEFAVALGHIARHRGFRSSAKRKAANTSGEDSKMLSALLVTRERTARYRTVGEMFARDPEYSARRRNRDGIFDRTVARDDLIHEVGKLFEAQRARGSAFASPDLEADFNAIAFRQAEMQDSERLVGNCPFERDEKRAARWAPSFERFRLLTRLINLRIATAEGERRFTPEDLARVSAELGATAKLPVSRVRALIGLLPDERFTTIRVEDEDRDIVARTGEAAPGTWRLRKTLGEALWAEMQARPDQLDAIAHVLSFFEATAKIRSELDRLGLPAAVIEALDRGLEDGAFAKFKGAGHISAKACRNLLPHLQQGHRYDQACRLAGYDHAASTLSHTAQVVSKAGFNALVTELGESIANPIARKALTEGLKQIRAMCARWGLPGAIHIELAREVGNSIEKRRELESQRETTTKRRQTERDEIRGLLALEDVDDDTLLRYRLWKEQGGKCLYTGKDIHVRQLIATDNSVQVDHILPWSRFGDDSFNNKTLCLASANQRKRGQTPWEWFSLTGTPEAWEVFASRVETSKDLRGFKKRNLLLKNADEVAQRFRSRNLNDTRYAARLLAEAAKLFYPAGDRQEKDGRRRVLTRPGPLTAALRHAWGLESLKKADGKRVSDDRHHALDALAVAAISEREVQRLTRSFQEWEQQGLARPLRHVAPPWPGFRGDVLAAYAQAFVARPERRRARGKGHDASIRQVVGEGPEAQVFERRGLADLKEADLDRVKDPERNQAIIEAVRAWILAGRPADAPPRSPRGDVIVKLRTAPAKKIKPAVPVRGGTADRGDMVRIDVFTRPNRKGRDEWYLVPIYPHQVMNRKAWPHPPMRSVVAYKDEAGWTEIGAEHSFRFSLYPRSYVQLVKSNGDVIEGYLVSMDRSTGAFRVFVHNNPAEMAKGIGAKTLLNLRKFNVDRFGVRTEVKSEVRTWHGEVCTSPVPPD

SEQ ID NO: 37 - protein, 1064 aa, Bacillus niameyensis
MEKLRRVLGLDIGVSSVGWGIIDQDSGDVIDAGVRLFEEAARNANEDRRSFRSSRRLKRRRTHRLERAKSLFEYNGLPSESIGKMDPYQARYNAIFGEVSKEELVAGLYHLVKRRGTTLDSPEDEKSSGNELSTKEQLSRNRKFLENRYICEVQLERLSNGHEKIRNHENRFRTEDYVKEARAILIRQQQRYSEITDEFIEDFINILKSKREYFDGPGSEKSPTPYGQYYLDKDGKLAYTSMINKMRGICTYFPDHLRIAKMSYTADLYNLLSGDLNKLLINGEYLTYEDKLYLIDHFIKKGKNITLTQILKYKGISDDSDVSGYRIDLKTDKPIFTEFTGFKELKKIVDKNDLPSSILEDIDLIDDIIEILTAEKSYYRREEQLKPLFSEYDQETIDRIVNAFKESTSFKGYHALSKKAIELILEDLWYTNKNQMELFAERKLEEKRLSDLKTQTKVMFDGDAILSTVAKRAHREAIKIVNEVRKRYGEMDAIVIETAREKNSEEKRKQYSDFQREIGKHEKEMAKLLGVKSLKDLRLNSKQHLALKLWVSQDRKCIYSGRSISINDIVQDPSKFEIDHILPVSLSFDDSQANKVLCYHRENQKKGQKTPFQYFSTGQASRSFDQFKVEVLNLYKSRKINNKKKDYLLEIRDIAHNEELQKEFINRNLVDTQYAMRSFSMSLRTFFNSHNIDTKVLSIRGSFTAAFRRRARLNKDREESHAHHAIDALIVAAIGRMPIFEFLKEFDMSEEGVVVNRETGEILEENEFFKHKYLSFIRNLMNYESKVKYSHKVDRKVNRTMSNQTIYGTREKDGDTYYLGKVSNIYNLDKHGVEPLLKRLEKSPNSFLIAEHNPKLFELIQLIVKEYKNADNPFKAYYNDHGYVMKDGKVPVKTLRYRDRKLGVHMKITDKYPGAKSDVVLLSIKGVRVDLYKNAEGKYKYLGVPYHWFKQQGNKYVLDMEKYNEEKMKDYKKIDDTFEFQMSLYKNDLFSFERKEGSYFRIFRGDAMPRDNKIEVDFIDKKKQDRKDGFLSPSTFKNIVKYNVDVLGNTYKVEKENFKNTLQL

SEQ ID NO: 38 - protein, 1134 aa, Enterococcus cecorum
MKYIESVSIGFDIGVGSVGYAVINSKTKQILESGVSIFPAAVAQKNVERRESRQSRRLRRRRVNRLNDLKKLLKKNGFIPSPNKNPYEIRVRGLEHQLSRDEIAIAVLHIAKRRGISYSLDDVVDENVTDFKGKIAKNQEALKNKTPGEIQYERLLKYGKVRGTVTNYVEYDEQPEYFLNVFPNSAYVDEAKRILTCQKQYYPEITDDLIENIVKIISRKRAYYIGPGNEKSRTDYGIYRTNGETLENIFEILIGKDSVNPDELRAPANSLTAQVYNLLNDLTNIRYNGSERLTPEQKQDILSELLTTEKTVQMLNVIAKKVNVKKEEISGFRTKKDDKPDFHTLEVYRKVRKHMLNKGIDINEWPTECFDELAFIFNLNTEIGEIRNRISEKVVPKYDFITNQIVDDLLEDPKIFAISTSQKWHRFSIKTMNVLIPELLNGYNEQMTILTRMGLLEDSKNDYSNIDKIDIKNVTENIYNPVVRKSIKKCLKIFNELYKKYPNISDVVIELPREDNPEDARKLIKKMQKENLVEKDTSLEAFRKEANLSENGLIDFLRKGKNRALKIRLWYQQEGKCLYTGEPILAEDLCKNEANYDIDHIIPISVSFDDSINNKVLCSSHFNRFEKEKKTPFEYFSNKSSHLGRYSNFDEFRASVNANKRISDVKRKNLLSKELLSDLDTRKKFIARNLVDTRYASRVVFNELTQFFASKKADTKVAVIRGKFTSQLRRAWQLNKSRDTYHHHAVDAALIGVTPFLKIWRSGKTIYPRKMTEAQTDFELNEITTNDEFKESYYQLPCPQFLDYLTQMQENLKINFEVDKKMNRKISDAMIYSSRKKKLSKDKKEKEYSLGTINDIYSDAGYSKFKNIYKKDKEKFLMYHLDHKSFEILEDIISQYPDKEEYIDSEGKVKQRAVSPFEIYRRKYGYIRKYSKKNNGPIIKNMKYYNENLPKIYIDITPKQSIDKKVYLTNISPWRTDVYYNRSQNSYHIMGIKYSDLRYIDGKYGIPKNVYEKIKLREKIPSESEFCFSLYKNDRVKIIDPNKNEEIELRFNSRTLPSVLNYVEFKPLFQESYNSLTLLPVFSKTTSEGRIIKKFTREDNIVYKVNTDELGNPYYILKESQNPKNIIDNSNKTE

SEQ ID NO: 39 - protein, 1091 aa, Amedibacillus dolichus
MGRLVLGLDIGITSVGFGIIDLDESEIVDYGVRLFKEGTAAENETRRTKRGGRRLKRRRVTRREDMLHLLKQAGIISTSFHPLNNPYDVRVKGLNERLNGEELATALLHLCKHRGSSVETIEDDEAKAKEAGETKKVLSMNDQLLKSGKYVCEIQKERLRTNGHIRGHENNFKTRAYVDEAFQILSHQDLSNELKSAIITIISRKRMYYDGPGGPLSPTPYGRYTYFGQKEPIDLIEKMRGKCSLFPNEPRAPKLAYSAELFNLLNDLNNLSIEGEKLTSEQKAMILKIVHEKGKITPKQLAKEVGVSLEQIRGFRIDTKGSPLLSELTGYKMIREVLEKSNDEHLEDHVFYDEIAEILTKTKDIEGRKKQISELSSDLNEESVHQLAGLTKFTAYHSLSFKALRLINEEMLKTELNQMQSITLFGLKQNNELSVKGMKNIQADDTAILSPVAKRAQRETFKVVNRLREIYGEFDSIVVEMAREKNSEEQRKAIRERQKFFEMRNKQVADIIGDDRKINAKLREKLVLYQEQDGKTAYSLEPIDLKLLIDDPNAYEVDHIIPISISLDDSITNKVLVTHRENQEKGNLTPISAFVKGRFTKGSLAQYKAYCLKLKEKNIKTNKGYRKKVEQYLLNENDIYKYDIQKEFINRNLVDTSYASRVVLNTLTTYFKQNEIPTKVFTVKGSLTNAFRRKINLKKDRDEDYGHHAIDALIIASMPKMRLLSTIFSRYKIEDIYDESTGEVFSSGDDSMYYDDRYFAFIASLKAIKVRKFSHKIDTKPNRSVADETIYSTRVIDGKEKVVKKYKDIYDPKFTALAEDILNNAYQEKYLMALHDPQTFDQIVKVVNYYFEEMSKSEKYFTKDKKGRIKISGMNPLSLYRDEHGMLKKYSKKGDGPAITQMKYFDGVLGNHIDISAHYQVRDKKVVLQQISPYRTDFYYSKENGYKFVTIRYKDVRWSEKKKKYVIDQQDYAMKKAEKKIDDTYEFQFSMHRDELIGITKAEGEALIYPDETWHNFNFFFHAGETPEILKFTATNNDKSNKIEVKPIHCYCKMRLMPTISKKIVRIDKYATDVVGNLYKVKKNTLKFEFD

SEQ ID NO: 40 - protein, 1142 aa, Facklamia hominis
MYSVGLDIGIASCGYSIINDVNGNIVELGTLLFKSRNSDNNEERRTNRGSRRLINRRRTRLDDGKKLLAEKGFKQDSKLKHVCPYELRVKGLHEKLTQGEIYKVVLHILKKRGISYLDESDENSSGIDGASYKDQVNLNLNLQKELTPGQIQLKRLKENGRIKSGVTFENGYQLNVFSVSAYANELNKILTKQSEYYSEIDQIWIDRFTKNGYRKEAGLVYRKRPYYDGPGNEKNNSIYGRWANFKEDGHPKENIFDQLIGLDSVDSTQVRASASTLTAQKYNLLNDLNNLTIIEDDRKFTTDEKVSIINYLMTQDITRFGPKELAKFYQLEVDQIRGWRIDNKGNPEIHSLKVYRDWKKIFNTSSIDLTSIQTWVVDELANVITLNTEIPSTLATLKIKIPDIDQQILNFVENNFYELKKKTSKNSWHSFSLKTLNILIPELLNTSEEQNTILERLNLKFDLKNKYAEKNQIPVKELLEQIYNPTVTKSVHQSIKLFNEIIEKYGKNNISYVTIEMPRDKNDQDQKDTIKRINKLNSERKNDSEKYFLERSGWDTIRFESALKKPRFAEKLLFYYEQKGKCAYSGQDILAESLLNESCEVDHIIPLSISFDDSIHNKVLVTASSNQEKGQRTPYMAFIDGAKLGQSWDDFVAWVNSCGFHRKKVQNLLYEENIFNPDVQRGFVSRNLNDTRYASRIVLNAIQSFFYKQKDLTKVKVVNGSFTHTLRKKWGSSLDKNRDTYHHHAVDATLCAVTPFINIVKYEFFEEDGIKYMVDLTTGEKVAYSDYKKMKFYDKRAYVPIWDGFIKQLTPANLYPKIKFSHQVDSKWNRKISDATIYSTRKIVETKINKKGVAKETVKDIVIDKIKDIYTPEGWRKFKEKQDKLLMKNFDEKTYNKLIKIAKEYPEKKEVQTSNGKVSLVEVSPFKMYCEEQNVPAIRKYSKKGNGPYIRSLKFYDKELGKHINITKDSYGNDIERTIRNKKIILKNQNSWRTDVYYNSVNNCYELLGIKYCHLKFYRGKYGIPKSIYDELKKEDEISSESVFKFSLYRHDRVRFEKDGEYIEALFGSKNESNKNYLELSPIDKNKWLSKENVKFFGEVAASGRFIKGLKPGIKVIKQSTDYMGHVYAIHQEKLKFVLSDA

SEQ ID NO: 41 - protein, 1063 aa, Massilibacterium senegalense
MLENYILGLDIGIGSVGYGLIDENQNIIDAGVRLFPEADVNNNNGRREKRGARRLKRRKQHRVARVKHLLVKNRIMAEDAQPISNKPYEIRVKGLSKELTDEELAIALIHIAKRRGIHNVETTLDEQKDDNELSTKEQISKNEKLLRNKFVCELQLQRLTRNKENEIPYKVRGYQNRFKTSDYIKEVQQILNTQRQYNKKITDEFIERYIELIKKRREYFEGPGEGSEYGWGGDPRKWYEQLMGKCSYFPEELRAVKASYSAQLFNALNDLNNIVIMREENTKLSAEEKRRLIEKVFKIRKKPTLKQIAKEVGVNFEDIKGYRVNAKGEPIFTSFDFYHSIKTITQRHEIIENDKILDQMARILTVMQTPSEKRERLKQEIPELTTSEISEISELRFTSTHSLSLKLIYLLLPELWSTTKNHMQILSEMKLKPKRISLKGRNQIPVSIVDEWILSPVVKRAFTQSIRIVNAIIKRYGEPSEIVIELARERNSEDRKKFLSKLNRKNAAMNEEVKEKIEQLGVYDTKGLFEMIKLWHLQDGHCMYSLTPIPLNDLFLNPNHYEIDHIIPRSISFDDSINNKVLVKYIENQKKGNLTPYQYMNRKIDGVSYDKFKQHVLQLAKSSDKMSRKKREYLLEERDINKFEVQKEFINRNLVDTRYVTRELMNLLKTFFSENNKNVKIKSINGSFTHYLRKLWRFKKDRNIDYKHHAEDALIIAMAGYLFSKKEVLAKQNLILSDPHIVNKETGEILSEDDFTKTFIDKYEKVATIKKYQNYKYSHRVDQKPNRQLMNDTICSTRTVDNNEYVIKKIKDIYAKDNTDLVNKIRKSPKKLLMYHNDRQTYEKIKSIMEQYGDAKNPLYKYKEETGEFVTKYSKKDNGPPILSVKYYSGKLGEHKDITHKFHNSKKRVVNLSIKPFRMDVYKEDGIYKFVTVRFNDLKEKGDNYEICKKIYEEKLKKKKINNKSNFLFSLNKYDIFELDNEEFLFIGVNHDKTNRIECNWINQNYKNFAELNNLKNKQLRKSISKNIIQFEKFATDVLGNRYKVTNEKLIFSYPKKSMRGVR

SEQ ID NO: 42 - protein, 1136 aa, Streptococcus gordonii
MNGLVLGLDIGIASVGVGILEKDTGKIIHASSRLFPAATADNNVERRSNRQGRRLNRRKKHRSVRLQDLFEGYGLLTDFSKVSMNLNPYQLRVQGMENQLTNEELFVALKNIVKRRGISYLDDASEDGGTVSSDYGKAVEENRKLLAEKTPGQIQLERFEKYGQLRGDFTVEENGEKHRLINVFSTSAYRKEAERILRKQQEFNSKITDEFIEDYLIILTGKRKYYHGPGNEKSRTDYGRFRTDGTTLDNIFGILIGKCTFYTEEYRASKASYTAQEFNLLNDLNNLTVPTETKKLSEEQKKLIIEYAKSAKTLGASTLLKYIAKMIDASVDQIRGYRVDVNNKPEMHTFEVYRKMQSLETIKVEELPRKVLDELAHILTLNTEREGIEEAINSKLKDIFNRDQVLELVQFRKNNSSLFSKGWHNFSIKLMMELIPELYETSEEQMTILTRLGKQRSKETSKRTKYIDEKELTEEIYNPVVAKSVRQAIKIINEATKKYGIFDNIVIEMARENNEEDAKKDYIKRQKANQDEKNAAMEKAAFQYNGKKELPDNIFHGHKELTTKIRLWHQQGEKCLYTGKNIPISDLIHNQYKYEIDHILPLSLSFDDSLSNKVLVLATANQEKGQRTPFQALDSMDDAWSYREFKSYVKDSKLLSNKKKDYLLTEEDISKIEVKQKFIERNLVDTRYSSRVVLNALQDFYKSHQLDTTISVVRGQFTSQLRRKWGIEKSRETYHHHAVDALIIAASSQLRLWKKHSNPLIAYKEGQFVDSETGEIVSLSDEEYKELVFKAPYDHFVDTLRSKKFEDSILFSYQVDSKYNRKISDATIYATRKAKLDKEKKEYTYTLGKIKDIYALGTKTPSKTGFYKFLDLYKTDKSQFLMYQKDRKTWDEVIEKIIEQYRPFKEYDKNGKEVDFNPFEKYRIGNGPIRKYSKKGNGPEIKSLKYYDILLGKHKNITPDGSRNTVALLSLNPWRTDVYYNSETKKYEFLGLKYADLCFEEGGAYGISEVKYKKIREKEGIGKNSEFKFTLYKNDLILIKDTETNCQQFFRFWSRTGKDNPKSFEKHKIELKPYEKAKFEKGEELKVLGKVPPSSNQFQKNMQIENLSIYKVKTDILGNKHFIKKEGDEPKLKFKK

SEQ ID NO: 43 - protein, 1130 aa, Streptococcus macedonicus
MTNGKILGLDIGIASVGVGIIEAKTGKVVHANSRLFSAANAENNAERRGFRGSRRLNRRKKHRVKRVRDLFEKYGIVTDFRNLNLNPYELRVKGLTEQLTNEELFAALRTISKRRGISYLDDAEDDSTGSTDYAKSIDENRRLLKTKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCSFYPDEYRASKASYTAQEYNFLNDLNNLKVPTETGKLSTEQKEALVEFAKSTATLGPAKLLKEIAKILDCKVDEIKGYREDDKGKPDLHTFEPYRKLKFNLDSVNIDYLSREVLDKLAGILTLNTEREGIEDAIKRNLPNQFTEEQISEIINVRKSQSTAFNKGWHSFSAKLMNELIPELYATSDEQMTILTRLEKFKVKKKSSKNIKTIDEKEITDEIYNPVVAKSVRQTIKIINAAVKKYGDFDKIVIEMPRDKNADDEKKFINKRNKENKKEKDDALKRAAYLYNGTDKLPDEVFHGNKQLETKIRLWYQQGERCLYSGKPIPIQELVHNSNNFEIDHILPLSLSFDDSLANKVLVYSWTNQEKGQKTPYQVIDSMDAAWSFREMKEYVLKQKGLGKKKRDYLLTTENIDKIEVKKKFIERNLVDTRYASRVVLNSLQSALRELGKDTKVSVVRGQFTSQLRRKWKIDKSRETYHHHAVDALIIAASSQLKLWEKQDNPMFVDYGNNQVVDKQTGEILSVSDNEYKELVFQPPYQGFVNTISSKGFEDEILFSYQVDSKYNRKVSDATIYSTRKAKIGKDKKEETYVLGKIKDIYSQNGFDTFVKKYNKDKTQFLMYQKDPLTWENVIEVILGDYPTTKKSEDGKNDVKCNPFEEYRRENGLICKYSKKGKGTPIKSLKYYDKKLGNCIDITPEESRNKVILQSINPWRADVYFNPETLKYELMGLKYSDLSFEKGTGNYHISQEKYDAIKEKEGIGKKSEFKFTLYRNDLILIKDIASGEQEIYRFLSRTMPNVNHYVELKPYDKEKFDNVQELVEALGEADKVGRCIKGLNKPNISIYKVRTDVLGNKYFVKKEGDKPKLDFKNNKK

SEQ ID NO: 44 - protein, 1127 aa, Streptococcus salivarius
MSDLVLGLDIGIGSVGVGILNKVTGEIIHKNSRIFPAAQAENNVERRINRQGRRLTRRKKHRRVRLNHLFEESGLITDFTNVSINLNPYQLRVKGLTDELSNEELFIALKNMVKHRGISYLDDASDDGNSSVGDYAQIVKENSKQLETKTPGQIQLERYQKYGQLRGDFTVEEDGKKHRLINVFPTSAYRAEALRILQTQQEFNPQITDEFINSYLQILTGKRKYYHGPGNEKSRTDYGRYTTKKDSEDEYITLDNIFGILIGKCTFYPEEYRAAKASYTAQEFNLLNDLNNLTVPTETKKLSEEQKNQIITNVKNEKAMGPAKLFKYIAKLLSCDVADIKGYRIDKSDKAEIHTFEAYRKMKTLATIDIEQMERENLDKLAYVLTLNTEKEGIQEALEHEFADGTFSQEQIDELVQFRKANSSIFGKGWHSFSVKLMMELIPELYATSEEQMTILTRLGKQKTTSSSNKTKYIDEKQLTEEIYNPVAAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEIDHILPLSITFDDSLANKVLVYATANQEKGQRTPYQALDSMDDAWSFRELKAFVRDSKALSNKKKEYLLTEEDISKFDVRKKFIERNLVDTRYASRVVLNALQEHFRVHKTDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKKDETYVLGKIKDIYSQTGYDAFIKIYKKDKSKFLMYRHDPQTFEKVIEPILENYPNKELNEKGKEVPCNPFLKYKEDHGYIRKYSKKGNGPEIKSLKYYDSKLGNHIDITPKNSNNKVVLQSVSPWRADVYFNKTTGKYEILGLKYADLKFEKGTGTYKISEEKYNDIKIKEGVDSDSEFKFTLYKNDLLLIKDTETKEQQLFRFLSRTMPNVKHYVELKPYDKQKFDDNEELIKILGIVAKGGQCKKGVSKPNISIYKIRTDVLGNQHIIKNEGDKPKLDF

SEQ ID NO: 45 - protein, 1122 aa, Streptococcus sinensis
MNGKILGLDIGVASVGVGILDKKTGEIIHASSRIFPAATADSNVERRGFRQGRRLGRRKKHRKVRLADLFSDTGLITDFSKVSINLNPYELRIKGLNEKLTNEELFIALKNIVKRRGISYLDDANEDGESSSSEYGKAVEENRKLLADKTPGQIQLERFEKYGQVRGDFTIEENGEKHRLLNVFSTSAYKKEAERILTKQQDYNQDITDEFIQAYLTILTGKRKYYHGPGNEKSRTDYGRFRTDGTTLDNIFGILIGKCTFYPEEYRAAKASYTAQEFNLLNDLNNLTVPTETKKLSEEQKRQIIEYAKGAKTLGAATLLKYIAKLVDGSVEDIKGYRIDKSEKPEMHTFDIYRKMQTLETVDVEKLSREVLDELAHILTLNTEREGIEEAIKVSFIKREFEQDQIAELVSFRKSNSSLFGKGWHNFSIKLMTELIPELYETSEEQMTILTRLGKQKTKARSKRTKYIDEKELTDEIYNPVVAKSVRQAIKIINLATKKYGVFDNIVIEMARENNEEDAKKDYVKRQKANEDEKNAAMEKAAHQYNGKKELPDNVFHGHKELATKIRLWHQQGEKCLYTGKNIPISDLIHNQYKYEIDHILPLSLSFDDSLANKVLVLATANQEKGQRTPFQALDSMDDAWSYREFKAYVRGARALSNKKKDYLLNEEDINKIEVKQKFIERNLVDTRYSSRVVLNALQDFYKLNDFDTKISVVRGQFTSQLRRKWRIDKSRETYHHHAVDALIIAASSQLRLWKKQGNPLISYKENQFVDSETGEIISLTDDEYKELVFRAPYDHFVDTVSSKKFEDRILFSYQVDSKYNRKISDATIYSTRKAKLGKDKSEETYVLGKIKDIYTQTGYDAFIKLYKKDKSKFLMYHKDPITFEKVIEEILKTYPDKEINEKGKEVACNPFEKYRQENGPLRKYSKKGKGPEIKSLKYYDNKLGNHIDITPDNSENQVILQSLKPWRTDVYFNHKTKIYELMGLKYSDLSFEKGSGKYRISLDKYNVIKKKEGVHKESEFKFTLYKNDLILIKDLEKSEQQLFRYNSRNDTSKHYVELKPYDKAKFEGNQPLMALFGNVAKGGQCLKGLNKANISIYKVQTDVLGNKRFIKKEGDAPKLEF

SEQ ID NO: 46 - protein, 1122 aa, Streptococcus suis
MSNGKILGLDIGIASVGVGVIDAQTGEIIHASSRIFPSANAANNAERRTFRGSRRLIRRKKHRIKRLDDLFNDFHINLDGEMSTDNPYVLRVKGLSQKLTVEELYISIKNIMKRRGISYLDDAESDNEAGRSDYAKAIERNRQLLTSKTPGEIQLERLEKYGQLRGNFTIIDEEGQSQQIINVFSTSDYVKEVEKILDCQKMYHKFISDEFCDKLIELLREKRKYYVGPGNEKSRTDYGIYRTDGTTLENLFGILIGKCTFYPDQYRSSRASYTAQEFNFLNDLNNLTVPTETKKLSQEQKEFLVNYAKETSVLGAGKILQQIAKLADCKVEDIRGYRLDNKDKPELHTFETYRAMKGLVPLVDIGVLSREQLDILADILTLNTDFEGIREALKKQLPNVFDEKQVKGLASFRKSKSQLFAKGWHNLSQKIMLEVIPELYATSDEQMTILTRLGKFEKSSVAEYPSSINVDEITDEIYNPVVAKSIRQTIKIINASIKKWDEFDQIVIEMPRDRNEDEEKKRIADGQKANAKEKADSILRAAELYCAGKVLPDYVYNGHNQLATKIRLWYQQGERCIYTGQPISIHDLIHNQNQYEIDHILPLSLTFDDSLSNKVLVLATANQEKAQRTPYNYLKSATSAWSYREFKDYVTKRKGIGKKKCEYLTFEEDINGFEVRSKFIQRNLVDTRYASKVILNALQDYFKISGIQTKVSVVRGQFTSQLRHKWGIEKTRETYHHHAVDALIIAASSQLRLWKKQESPLVVDYQEGRQVDLETGEILELTDEQYKELVYQPPYQGFVNTISSSAFDNEILFSYQVDSKVNRKISDATIYATRNAQLGKDKTEGIYVLGKIKDIYTQAGYEAFLKRYTKDKTSFLMYHKDLDTWEKVIEIILRDYREYDEKGKEIGNPFERYRRENGYVKKYSRKGNGTAIKSLKYYDNKLGNHIDITPENSRNAVVLQSLKPWRTDVYFNKETGKYEFLGIKYSDLSFEKGTGEYGISQEKYDSIKIAEGVAKKSIFKFTLYKQDLLFIKDIENNFGKLLRFTSKNDTSKHYVELKPYDKNKFGTEEPLLPVLGNVAKSGQCIKGLNKSNISIYKVRTDILGYRHFIKQEGEHPQLKFKK

SEQ ID NO: 47 - protein, 1107 aa, Turicibacter sp.
MGYTLGLDIGIASVGFGLIDNVDGKILESGVRLFSSADPDNNQTRRSSRQARRLNRRKVHRLERVAHLLEEAGFEKGEVLAPTPYHLRVKGLTEKLSKPEVYAALYNLMKHRGISYLEDVELDEADSSSVSQNVELLKEYYPCEIQLERYEEYGQIRGIIETYNKDGEKESLVNIFPTGAYKKEAEAIILKQGQFYPELDKVFLEKFTEILTVKRPYYVGPGNKKSRTDYGIYRENGETLENLFDILIGKCTIYPQEARAAKFSYTAQLFDFLNDMNNISIAGEKLTEVQKREIYHQIMSSKTVNMMKIITKVTGCLKEEVTGYRVDSKYKAEFHSFASYRQFRNKMLDYGFDVQTLNVEMFDKLAAKLTLNTEYHQIITQLESLNLGLSKELIEKIADFRKKNRSLFSKWHSLSLKAMNEVVDELWSTSKNQMQIFSELGGFKSNIEVFKGLKYIPLDAMVDEIYNPVVRCSIRESIKIINALLKKYGALDKVVIEMPRDSNEKEEKDRITKLNKENDNKKKQAIEKARNEYGFGEEAYRQQNDLNTKLRLWYEQKQRCLYSGEIIKIFDLIKNPHLFDIDHIIPKSISYDDSLSNKVLCYATENREKGNLTPYRYFKRKASLTWNYEAYKSYVIELYKDGKGDLSRTKLELLLFEEDINKYEVRQRFINRNLVDTRYASKVVLNTLQDFMKENYPTTTVEVVRGKFTAQLRRHWGIRKNRDENHAHHAIDALIIACVPHLSLWKEQKKVFSTDEFRVDETTGEIIEQSYNQLTYQEPFSQFFYQLEKVSIDCRYSYKVDRKFNRQVADATIYSTRQGIVKQTSGDKSFTLDSNSKEHYVIAKIKNIYEDVDAKRFMDRYKKDKTQFLMYHHDPQTFSILEDVMKTYPDKKNPFQAYRLEYGPIRKYSKKGNGPQIQSIKYYDSKLGQHIELKQANQSTKKVVLQSLNPWRCDIYYNKKDKRYIAAGLKYADLSYQKGTGLYGISLQRYQQILNSEKIELSIEQIELLLEGRLNESNFEFCFSLYKNDFIQWNDENNQTFQYRFLARNLSSPNRIEVKPINKSKYDKQAEGLKTLKKGLNRFYKISVDVLGYQHFIKKEKLKLTFK

SEQ ID NO: 48 - protein, 1074 aa, Halalkalibacter okhensis
MTDTRDYRIGLDIGTNSIGWAIIELEYNGEQYDKKGLIDAGVRMFDKAEIPKTGASLAEPRRLARSTRRRLNRRADRKKSIRRLIVEQDILNEAELQVLYQEKELLDIWTIRVEALDRRLSRTEWARLLIQLAQRRGYRSARKSEENDKETGVVLQNIRENKKLIDGYRTVGEMWVKDPRFQGRRHNSRSEYVFNVTREMLEEEIRTLFHTQRELGLSFASKEMEESYLSIWGKQLPFASGKDILKRVGFCTLEEQERRFPKATFTFQYFMVLDKLNRLRIKPSMRPLTEEERKSTLHYLFDRDDLSKKKTPPKVMYKEIRKQLNLAATETFNELTYDPNESMTKNENVLFVNLDAYYRIKQQCDLHNERYGPRDYDALGYALTVYKTDKDIRQYLRNKTNLANRIYSEELISNLLSQSYATFGHISKKALLAILPSMEKGDTFKVAADKMGYDTTGLQLVKKELLLPVIPNDITNPVVMRALTQARKVVNAIIKRLGEQPLSIHIELARDLLKGHDERRKISNEYKQNHKRNAGAYTILNENGIINPRGYDIVRYKLWQEQKQKCAYSGKVIPSETFFGELKKERGQGAPTLDVDHIIPYSKCFNDSYSNKVLVYSDENRKKGNHLPFDYLKESPSRWEEFETYVLTNEFPRAKRERLLKKQFSEDEESVMKERHLNDTRYASRYFKQFVEKSLQFKKSKNQFHKKRVETVAGRVTAHFRQRWGFEKHREGTHLHHALDAIVVACTDAAMIQALSLYHKQKEEGKKGYIKRFPLPWITFRDDVLNKLSESAIPEQIYSALVSGTTLPSYMLVSRMPRRSVTGPVHKETISKDGGVDEKGKTLIVKKVQLQDIKFNKNGDFLMYMEETDPATYKAIKERYLSFNKDAKKAFQDPLYKPSKKGKPNQIKSVKVIVEKKAHVRKVNGGVAENGDLIRIDFFQKEGKFFMVPLYTYDTIQSDLPNRYVVSGKGYQLWPQLTNEHKFIQSVHPYELLRVVKRSGDDRFLYLASINISNNVLKFIEPNFPHNDYIEISLGTIKHMEKYDVNVLGQVSLKATGPRLGFNNMKAQKNKVKG

SEQ ID NO: 49 - protein, 1001 aa, Campylobacter coli
MKILGFDIGINSIGWAFVEDNQLQDCGVRLFTKAEDPKTRESLALPRRNARSNRRRLGRRRSRLIALKHIISKELKLNYQDYIANNGELPKAYEGRLISPYELRYKALNEKIEPKDLARVILHIAKHRGYMNKNEKKSSDNEKGKILSALKTNALKLEKYQSVGEYFYKEFFQKYRENTKDFINIRNKEGSYENCVLASDLEKELKLILEKQKEWGYSYNDNFIKEILKVAFFQRPLKDFSYLVGACTFFEDEKRACKNSYSAWEFVALTKIINELKSLEKESGELVSSQIINEILNHVLDKGSITYKKFREYIKLHESMKFKSLKYDKDNVESTKLIEFRKLVEFKKALGEHSLSREELDQIATYITLIKDNEKLKITLEKYSLNNEQIKNLLEIDFNDHINLSFKALNLILPLMKEGKRYDEACKLLNLKTKSNNQKFDFLPAFCDSIFAQELTNPIVNRAISEYRKVLNALLKKYGKMHKIHIELARDIGLSKKLRAKIEKEQKENYENNIWALNECENFGLKANAKNILKLKLWKEQKEFCIYSGKKISIEHLRDEKALEVDHIYPYSRSFDDSFLNKVLVFTKENQEKLNKTPFEAFGANEERWSKIQALAQNLPYKKKNKILDEAFKGKQQQDFISRNLNDTRYISTLIVKYTKEYLDFLPLDEKEDINLKSGEKGSKIHVQTINGMLTSVLRHTWGFAQKDRNNHLHHALDATIVAYSTNAIIKAFSDFKKEQELLKAKLYAKELTNDSYKHQAKFFEPFEGFREQILNQINKLFVSKPPRKRARGALHKETFYPKDEMIKKYNSQEGVEIALNCGKIRKIGTKYVENDTMVRIDIFKKQNKFYAIPIYTMDFALGVLPNKIVIAGKDKKGNPKQWQEIDESYEFCFSLHKDDLVLIQKKDMQEPEFAYYNSFDIGNSSICVEKHDNKFENLTDNQKLLFVNAKEGSVKAEKLGIQGLKIFEKYIITPLGEKIKADFKPREDIALKTSKKHGIR

SEQ ID NO: 50 - protein, 1115 aa, Dechloromonas denitrificans
MSATYTIGLDIGIASVGWCAFGETHILDLGVRAFDKAETAKEGESLNLARRNARLMRRRLFRRTWRLKKLARQLKSEGLIADTKLFKPELPFSKSLWQLRVDGLDRRLNDEEWARVIYHLCKHRGFHWISRAEAKQAEGDSKSEGGKVKQGLAGTAKLMDEKGYRSAAEMVLAEFPQAQRNKQGEYQKALSRELLAKELALLFAQQREYGNLHTPGALETAILGNGDKKSGLFWQQKPALAGTDLLKMLGTCTFEKGEYRAPKASFTAERHVWLTRLNNLRIVTDGVTRQLTEQERQLCLPLPYRQAGDLTYKQLGSALAKAGLLEAGGFKFTGLAYPTEAQKAEGKAKDPESATLVKIPAWHELRQTLTKGGLNDEWEKLAGDALCGQPEIFDKIAWVLSVYKDDDEVRAELAKLALPNPQALTEALLNIRFDKFHSLSLKALRAIVPHMEQGLRYDEACVQAGYHHSQLLKVGEGEHKYLPPLYDGRDGDGRMIFAEDADIPRNPVVLRSLNQARKVVNAIIRRYGSPSAVHIEMARDLSRPFDERNKIKKDQDEYQARNQRDREQFSELFGHPPKGLEFEKWRLYREQNGQCAYSQRPLTASGNVAEIFLDGSTEIDHALPYSRSFDDSKNNKVLVLTRENRDKGNRTPYEYLGGAENSESWQRFVSFVESNKAYRLAKRTRLLRKDFGAKESEEFRERNLNDTRYVCRFFKNYVEQHLKLAEGSETKRCVVVSGQLTNFLRARWGLLKVRSDSDRHHALDAAVVAACSHSMVQRLSNYSRTRELEQVRDGFVDIETGEIVNPAMHQQLREHFPDPWPYFRHELEARLKIDSPELLREEMARLGSYSEEELKVLKPLFVSRAPQRRNGGAAHKDTIYGQPEKLKKNGSVTQKVALTSLTLKDIDLLADPHRNAKLYGAIRERLEAHGGKGDKAFPATNPLRKPDKEGNPTGPIVRTVTKVIDKLSGIPVRGGIAKNDTMLRVDVFTKGGKFHLVPVYVHHRVTGLPNRAIIAFKDEDEWTLIDDSFDWCFSLYPNDLLRIKLKKESHSGYYSSCNRSTGAVNLWAHDRNVNVGKDGFIESIGVKMAVSIEKLHVDTLGNIYPAPPEKRRGLA

SEQ ID NO: 51 - protein, 978 aa, Campylobacterota bacterium
MSKMILGLDLGITSIGWALVNVDDEDAQNNKIIDSGVRIFTIAEHPKDGKSLALPRREARGARRTTKRKAQKIKAVKRLLIKEKVVTANELNNLFIGNKKQKDVWDLRREALYRELNNKEFARIMVHLAKHRGYASNRKSEEPTDSEGKAVLSGITHNKTILQEKSYLTIGEYISTKTKKRNGKDTEGKLNYENSVSREMLINEITTIFERQKEFSNPHAKEELLEEYKNIAFTQRALKSVEGMVANCPFETNKKRAPKASSTFETFRALQKLKNLRMISNEGESALTKEQILQAIQKAKTAKSFTYKTLKTLFALPKEVQFKGLTYFDHKTGEAKEPESAKLLDFSAYQKIKDAIQSSDNLYWNKIEESVNLLDEIAKILSTHKDDKKSLEELNKIIDNPSVCNALISLSFSGFGHISVEAMRKINPYLLEGIDYDKACEMAGYDFKAIFKGDKSTLLPPLSEQENLEMTNPVVKRAIAQMRLVYNAITRKYGAIDAVHVEFTRDIKKSHKDRNDIKKSQEEFQALKEEAKAEAIKILGKEPSAKELLKFRLWKEQNGECIYSGAYISPSILADPYATEVDHILPYSRSLDDSLNNKVLCLTKQNQDKGNKTPFEYFGADKDSKEWQEFTGRVEGLKNIKGAKKGRLTKTNFDESSENAFKERNKNDTSYISKFVKNYIESHVEFKESKMKRHVYTMNGMLTSQLRYKWGVGDKNRENHLHHAEDAIILAFATQAQVQKLSTTSAKREGFSFKNKEEKAKELRFEPPMENFNEKVKESIENIFVSHMPRRKVSGSAHKETVYSNKTHSVKKADGKVEYLKGNSVNNNVKLKHGIALNDSMPRVDLFQNKKTNKYYLVPIYVSDFVQETLPNKAIVAGNKPWLEMDEEYEFKFSFYKSDLIEVKTKKTATKESVQVLGYFNTAVSSTAQVVIDTHDGNEEYRYGTQNLIFVKKYQVDALGNYIEVKNEKRQGTKKQNR

SEQ ID NO: 52 - protein, 1087 aa, Geobacillus thermopakistaniensis
MRYKIGLDIGITSVGWAVINLDIPRIEDLGVRIFDRAENPQTGESLALPRRLARSARRRLRRRKHRLERIRRLLIREGVLTKEELDKLFEEKHEIDVWQLRVEALDRKLNNDELVRVLLHLAKRRGFKSNRQSERSNKENSTMLKHIEENRAILSGYRTVGEMIVKDPKFALHKRNKGENYTNTIARDDLEREIRLIFSKQREFGNMSCTEEFENEYIAIWASQRPVASKDDIEKKVGFCTFEPKEKRAPKATYTFQSFIAWEHINKLRLISPSGTRGLTDEERRLLYEQAFQKNKITYHDIRTLLHLPDDTYFKGIVYDWGESLKQNENIRFLELDAYHQIRKAIDKVYGKGKSSSFLPIDFDTFGYALTLFKDDADIRSYLRNEYEQNGKRIANLANKVYDNELIEELLNLSFTKFGHLSLKALRNILPYMEQGEVYSSACEKAGYTFTGPKKKQKTVLLPTIPPIVNPVVMRALTQARKVVNAIIKKYGSPVSIHIELARDLSQTFDERRKTKREQDENRKRNETAIRQLMEYGLTLNPTGLDIVKFKLWSEQNGRCAYSLQPIEIERLLEPGYVEVDHVIPYSRSLDDSYTNKVLVLTKENREKGNRIPAEYLGVGTERWQQFETFVLTNKQFSKKKRDRLLRLHYDENEEMEFKSRNLNDTRYISRFFANFIREHLKFAESDDKQKVYTVNGRVTSHLRSRWDFNKNREESDLHHAVDAAIVACTTPSDIAKVTAFYQRREQNKELAKKTEPQFPQPWPHFADELRARLSKHPKESIKALNLGNYDDQKLESLQPVFVSRMPKRSVTGAAHQETLRRYIGIDERSGKIQTVVKTKLSEIKLDASGHFPMYGKESDPRTYEAIRQRLLEHNNDPKKAFQEPLYKPKKNGEPGPIIRTVKIIDTKNQVIPLNDGKTVAYNSNIVRIDVFEKDGKYYCVPVYTMDIMKGVLPNKAIEPNKPYSEWKEMTEDYTFRFSLYPNDLIRIELPREKTVKTAAGEEINIKDMFVYYKTIDSANGGLELISHDNSFSLRGVGSRTLKRFEKYQVDVLGNIYKVRGEKRVGLASSAHSKTGETIRPLQSTRD

SEQ ID NO: 53 - protein, 1069 aa, Neisseria sp. 83E34
MKKQPQRYILGLDLGIASVGWALILVDGAENPVGLLDCGVRTFERAEVPKTGDSLALARRQARSVRRLIRRRAHRLLRLRRLLKREGILSAADFNSDGLPNLPIDAWQLRVAGLERKLTNQEWAAVLLHLVKHRGYLSQRKSETQTENKELGRLLAGVAENSKLLSESSEQYRTPAELAVKLFAEQSGRLRNKGGDYTHTFNRLDLQKELHLLFERQCALGNPFASAELQSKVDELLMTQRSALQGEAVLKMLGKCTFEPSEYKAAKNTYSAERFIWLTKLNNLRILYNGEDRALDEGERKQLLDEPYKKAKLTYKQVRSILNLPESAVFKGLRYGKDSDGLKAENGALMEMKAYHKIRTVLEKAGLKGEWENRIRNPEILDQIGTAFSVYKTDEDIKAELAALRLPENVLDALLAGLNFDKFIQLSLKALSNILPLMERGIRYDEACKEVYGDHRALAESKNQMLLPQIPANEIRNPVVLRTLTQARKVINAIIRRYGSPQRVHIETARELGKSFKDRKEIEKRQEENRKERERAAEHFKELFPRFTGEPKSGDILKLRLYQQQQGKCLYSGIPIDLGRLNEKGYVEIDHALPFSRTWDDSFNNKVLVLGSENQKKRNQTPYEYLDGARDSQRWREFQARVQGCRFPYAKKQRIQTAKLDSKTEQGFLERNLNDTRYIARFLCGFIEKSLHLQGAGKRRVFASNGQITALLRGLWGLKKVREDNDRHHALDAIVVACSTVSMQQKITKAMQRRETLETINTETGEVKTRIPQPWDFFRQEVMIRVFSDAPREELAEKLNSRPEALHDFVSPLFVSRAPNRKMTGQGHLETVKSAKRLSENISVVKKPLTALKLKDIENIVGYPDREPALYRALQERLQAYKDDPAKAFAEPFYKPAKDGGQGSLVKAVRIIENTKHGLKVRNGITDTNGDMVRADVYQKADKKGKRQYFIVPVYAWQVEKNELPTVDCKGYEIDDTYEFCFSLYKYDIIEIQNPDNMSKELRVYYINCDSSDGRFWLAHHDKSAKEQQFRISTQNQPIIKKYQVDPLGKEIRLCKAEKRTELNGKKKK

SEQ ID NO: 54 - protein, 1137 aa, Nitratifractor salsuginis
MEDRIVKKILGVDLGITSFGYAILQETGKDLYRCLDNSVVMRNNPYDEKSGESSQSIRSTQKSMRRLIEKRKKRIRCVAQTMERYGILDYSETMKINDPKNNPIKNRWQLRAVDAWKRPLSPQELFAIFAHMAKHRGYKSIATEDLIYELELELGLNDPEKESEKKADERRQVYNALRHLEELRKKYGGETIAQTIHRAVEAGDLRSYRNHDDYEKMIRREDIEEEIEKVLLRQAELGALGLPEEQVSELIDELKACITDQEMPTIDESLFGKCTFYKDELAAPAYSYLYDLYRLYKKLADLNIDGYEVTQEDREKVIEWVEKKIAQGKNLKKITHKDLRKILGLAPEQKIFGVEDERIVKGKKEPRTFVPFFFLADIAKFKELFASIQKHPDALQIFRELAEILQRSKTPQEALDRLRALMAGKGIDTDDRELLELFKNKRSGTRELSHRYILEALPLFLEGYDEKEVQRILGFDDREDYSRYPKSLRHLHLREGNLFEKEENPINNHAVKSLASWALGLIADLSWRYGPFDEIILETTRDALPEKIRKEIDKAMREREKALDKIIGKYKKEFPSIDKRLARKIQLWERQKGLDLYSGKVINLSQLLDGSADIEHIVPQSLGGLSTDYNTIVTLKSVNAAKGNRLPGDWLAGNPDYRERIGMLSEKGLIDWKKRKNLLAQSLDEIYTENTHSKGIRATSYLEALVAQVLKRYYPFPDPELRKNGIGVRMIPGKVTSKTRSLLGIKSKSRETNFHHAEDALILSTLTRGWQNRLHRMLRDNYGKSEAELKELWKKYMPHIEGLTLADYIDEAFRRFMSKGEESLFYRDMFDTIRSISYWVDKKPLSASSHKETVYSSRHEVPTLRKNILEAFDSLNVIKDRHKLTTEEFMKRYDKEIRQKLWLHRIGNTNDESYRAVEERATQIAQILTRYQLMDAQNDKEIDEKFQQALKELITSPIEVTGKLLRKMRFVYDKLNAMQIDRGLVETDKNMLGIHISKGPNEKLIFRRMDVNNAHELQKERSGILCYLNEMLFIFNKKGLIHYGCLRSYLEKGQGSKYIALFNPRFPANPKAQPSKFTSDSKIKQVGIGSATGIIKAHLDLDGHVRSYEVFGTLPEGSIEWFKEESGYGRVEDDPHH

SEQ ID NO: 55 - protein, 1095 aa, Corynebacterium camporealensis
MQKNYRVGIDVGTHSIGFAAIEVDDHDRPIHILSAMSLIHDSGLDPDSNKRALTRLAESGVARRTRRLYRRRRKRMVRLEKFLQHQGWETKPFEDYSDPYIPWNARVALVNGYIEDDQQRGEYLSIALRHIANHRGWRNPYWKTRSLYSPEGPSDAFETIRKDLEKRAGRPVPKDITVGQLITFARIGTDRIRGEAQNKKTGHNNPAILSARLHQLDHAREINEIAKVQKLDDDLRKHIIDLVFAAESPKGAQLGRVGKDPLQPGKDRALKATDAFQRYRIAALTGNLRIRGTQGNDRLTREQRQLIFDHLVNLPHTEEPSWLNIAEILGIDRGDLLGTATMTDDGERAGSKPPVHDTNRAILSSKVKPLVHWWKAANFDEQKAMLKSLSNAEVDDFDSPAGASVQAFFADLDEKQHDKLDSLHLPMGRAAYSEDTLVRLTERMLNNNQDLYEARLAEFNIAKDWTPPKPRIGEPVGNPAVDRVLKATARWLEAAHKEWGAPKSIIIEHVRDGFISEAKAREIDRDNNRRAQRNRELFGKMQSKLGIEGKPRSADLWRFQSVQRQNCCCAYCGDTITFQNCEMDHIVPRAGEGSTNTRDNLVAVCHRCNLAKKNIPFAVWAKNANIPGVSLKEALNRTRHWVEDTGMSSKDFQRFVSNVRARLKRTSVDEPLDNRSIESVAWMANELRSRVAQHFGEYGTKVGVYRGELTAEARNAAGIRRKLRFIDGTGKSRLDRRHHAVDAAVICFTQNYVAEVLAKRSSMRRAAQLNREPEQWREFTGSTDAHRAAWNAWVNKMHLLANLLQTALDEDRIVVTSNLRLRLGNGRAHEDTIGELKKIRVGDAISVTDIDRASTEALWCALTRDPDFDPKEGLPENPHRRIRIHGTWYDADDHIEVFPVGAGAIKVRGGYAELSRFHHARVYKITSGKKPTYAMLRVYTTDLTAYRNEDLFSVEIKPQTMSMRQAEPKLRKAIAEGQAEYLGWIVIDDELLIDTSSFNSGQIAEMQAEFGMVRRWRLDGFVSPSKLRLRPLQMSGEGLAPGASAGSRKIVDQPGWRPAVNKLLSAGNVTIIRRNSLGQVRLNSNAHLPTTWKVD

SEQ ID NO: 56 - protein, 1028 aa, Ferrovibrio sp.
MRIWGFDVGTTSIGFAVIDYDSDRKAGRIERLGVRIFPEGVTEDKGEPRNAQRRAKRMMRRQLRRKRWRRRALHEILAEAGLLPTDDPKQWPAELAQADPYDLRDRGLSQKLSPHEVGRALFHILKRRGFQSARIAAVEPSDPAKEKEDEGIKAEISQIAAELQGRTLGQWLKSQSKRRGHHTARTMTQEEFERFWTAQVKYHPDLLTDNLRAALAETAFRQRPIFWRLKTLGTCLLEKDAPLCPKGAWIGQRFEMLQTLNNLRLAGGNARPLDEDERRLLLAALETSPSLSFPAARKLLKPVWQRNGTSLKTKFNFEVEGGKKALPGNALEAALIDAFGEAWNTHPKKDAIRAELQDRLWAIQYRKLGEKRVEIRDGNSAEAERQRFIAFAQAEWNASRKTAETLSRIEPPAAWLRHSIKAIQKLLPHMEAGLTYAEARKLVYPEQFEKSDPIEKLPSDPRQFEDLRNPTVKRALTELRKVVNNLLATYGKPERIRIELARNVALTGKDKANYMRTLAERERQRKQAAKELKASLGREPTAGDIEKWLLWQECEKTCPYTFDSIGFADLFGNMPRYDIEHIWPRSLSLDDSFANKTLCRRDVNNKKGQRTPFELYASNHGIADLPDWEEVKIHLRNKVDAGHFPESKWKRFTREQPFAAADDRSDDRLIGDTAYIAREARAWLGQLMPRGIFDVEPRNGRIVSRLRKLWHLDRLLGEDGRKNRDDHRHHALDAAVTALFDTRAMQDIVRWYKAQEDRPEEKPPAIDLPWPAFRDDLRTALEAIVVSHRVRRKVNGPLHDETRLGDSGEPPIREGGKEYRFYVKRKALAQMSKGEIDAIRDERIRTLAQERLAAHDGDVKKAFATPLMLPGRAGKPDREVKKARVIVKSQPHLVTALNGGKAYAAASDNHHMAIYRSIDGKVSYRTVSRIEAARRTARKEPVVQRVLPDGAQLLFTLTPGTTLAFPNNNGRPIYRVVTSVWDSGQIVLVDHTEAGGTVWKRPNASSLVAMGAKKVSVDPIGRVRPARD

SEQ ID NO: 57 - protein, 1044 aa, Mucispirillum schaedleri
MAEKKFKYRLGLDLGTNSIGWCMLRLNKDNEPAAVIKSGVRIFHDGRNEKTKEPLAVVRRNARSIRRNLDRKISRRNHLLNTLVKYFLLPSNEIERKALAVLNPYELRSRAVNERLELFELGRLLMHLSKRRGFKSNRKVDKEDSSGKIKPAIERLKNKMQETNKKTAGEYLYSLMQNGKPVRARLGRIDGSNGYEIYLDRALIEEEYNLIMNEQKKHHKELTDDIINEIFNIIFYQRPLKKQIIGKCSLTGEDTKLKKAHVLAQDFILLQKVQDLRVFDDNQLEFRPLSNDEKDIIKRELRKKKEVSYENIRKLLKKYYENVKYGMFSHETLYKKLQGNKTNALMSDKEIIGDSWYSLSPEVQYDIIEELYSDKDNDELSKLYSEKFNLNESQINGLLNKAIYRLDSGYIRYGKTAVTNLNSIMDKDNLNLHDAREEAGYEKENKQDASEYLEYYGKVLPDSVVDPQIENPKNDEERYGKIANPTVHAALNQLRKLVNELIKLYGKPEQIVIELARDLKNSRKAKDVINKKIAENRKLNEEVDKTLQEYSEKYNTHIVKNKLNRDKVKLWRELGKDPLERKCIYTGEQINIAKLFSDEVQIEHIVPYSKTLDDSFNNKTLSMKQANYYKGNKTPYEAFKDSKDGYNYDDIIARAKLLNNDNKFKRFTKDAMDIYNRDGKDFIARQLSDTQYLSTRALIYLQSLYEKAHHSSIWTIPGQLTALIRNYLGLNTLISNDKNKNRNDHRHHAVDAFVVAVTSRSLLQKISYAASLQEDLDNRNVSSYRNKLLEKMPEPFPNYRKSLELAIDNIKTSHKADRSISGKLHEDTAYGIVENDDKYNVVTRWTVDKFKEQKHFETIRDNELKEIACSDKELFAKIVETRKIKSIRKLAKENPVIKIKDKSGRDYKAFAGGNNICVEIYEVDGVRKSEVIQLFDAAQKGFMPEWMKKYPNGKLLMRLFKGDVIGFIENRKYNYYTIKGISIADQNLKLLAINKAEGIFKKGLNILFDSNNKNSLNAKQFNISVTGIVTKKKTADVNFWKNRK

SEQ ID NO: 58 - protein, 1070 aa, Sulfitobacter donghicola
MRLGLDIGSTSIGWWIYETTDGQITSVIDGGVRIFSDGRDPKSKGSLAVDRRVARAQRRRRDRYLRRKAALMKRMAEVGLMPSDPKQAKQLEALDPYELRARGLDETLELTELGRALFHLNQRRGFKSNRKTDAGDNESGKIIDATARLDWAMRDHRARTYGEFLHMRRQKADTPRTTPAVRTRLTIAARDGAEKEESGYDFYPDRQHLRDEFDALWEAQAEYHGVLTEDLRDEIGTIIFHQRPLKTPKVGKCLFTNEERVASAHPLNQRRILVETVNALRVAQLGEVARALTLEERDTILHALDNKAHTKSLSGMSIKLKALGKVIKLRSGQYFTLETANRDAIACDPVRASLSKPDRIGSYWSTLDHNAQWEVVNRIRAVQSDADHRELVAWLVSQHRLAAEHAEGAAYAPLPDGYSRLGITATKRILAALEANVIPYSKAVESCGWHHSDERTGEVLDRLPYYGEILDRHVIPGTYDDSDDDITRFGRITNPTVHIGLNQLRRLVNRIIEVHGKPDEIVVELARDLKLSQDQKADVERTIKKNTQAAIRRGEKLEEMGVPNTGGNRMVLRLWEDLNDDDVMKRFCPYTGQRISAGMLFDGSCDVDHILPFSRTLDDGFTNKTLCLKEANRKKRNYAPHEAAEKGLLDRDAMDSVAKYLPDNKAWRFAPDAIERFEGERDFTARALKDTQYLSRLARTYLDALYDGGDDKSHVWVVPGRLTEMLRRHWGLNGLEALADSDEKTVKRKNRTDHRHHAIDAAVVAATDRSLIQKISKIAASDEQAGAEDTLKSTPPPWDSFRDDIAAQLQNITVSHRADHGRTDSLAKGRGEDTTSGALHEATALGIIDETHVTVRIPIESLKSGHLIEGSRNGYIRDPYLRDQLTLATKNGSDKDFTTVLQKFFDRSKRYGGTRRVRIIKTLQDTARISVPHNAPIKAYKGGSNHAFEIWRLPNGKIESQVITTWEHHNLTEPKRPHPAAKRIIRLHKTDMVAIERDGKTVICFVQKTDPSNGAFLVPHFEANADGRKRKNEPVEWYQMSANSLIKADLRRVGVDEMGRVRDPGPPPIR

SEQ ID NO: 59 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHRETLRRYVGIDERSGKIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 60 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKAINSANGGLELISHDHRFSLRGVGSRTLKRFEKYQVDVLGNIYKVRGEKRVGLASSAHSKPGKTIRPLQSTRD

SEQ ID NO: 61 - protein, 1082 aa, synthetic construct
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEGNQSILSSYRTVAEMVVKDPKFSLHKRNKEDNYINTVARDDLEREIKLIFAKQREYGDIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIQPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHRETLRRYIGIDERSGKIQTVVKKKLSKIQLDKTGHFPMYGKESDPRTYEAIRQRLLEHNNDPKKAFQGPLYKPKKNGELGPIIRTIKIIDTRNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 62 - skipped

SEQ ID NO: 63 - protein, 1441 aa, synthetic construct
MKSSHHHHHHGSSENLYFQSRAAGSSGSPAAKKKKLDNATSDKKYSIGLAIGTNSVGWAVITDEYKVPSKKFKVLGNTDRHSIKKNLIGALLFDSGETAEATRLKRTARRRYTRRKNRISYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIESFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGDAGSPKKKRKVDGSGSKRPAATKKAGQAKKKKLE

SEQ ID NO: 64 - protein, 7 aa, synthetic construct
PKKKRKV

SEQ ID NO: 65 - protein, 16 aa, synthetic construct
KRPAATKKAGQAKKKK

SEQ ID NO: 66 - protein, 9 aa, synthetic construct
PAAKRVKLD

SEQ ID NO: 67 - protein, 11 aa, synthetic construct
RQRRNELKRSP

SEQ ID NO: 68 - protein, 38 aa, synthetic construct
NQSSNFGPMKGGNFGGRSSGPYGGGGQYFAKPRNQGGY

SEQ ID NO: 69 - protein, 42 aa, synthetic construct
RMRIZFKNKGKDTAELRRRRVEVSVELRKAKKDEQILKRRNV

SEQ ID NO: 70 - protein, 8 aa, synthetic construct
VSRKRPRP

SEQ ID NO: 71 - protein, 8 aa, synthetic construct
PPKKARED

SEQ ID NO: 72 - protein, 8 aa, synthetic construct
PQPKKKPL

SEQ ID NO: 73 - protein, 12 aa, synthetic construct
SALIKKKKKMAP

SEQ ID NO: 74 - protein, 5 aa, synthetic construct
DRLRR

SEQ ID NO: 75 - protein, 7 aa, synthetic construct
PKQKKRK

SEQ ID NO: 76 - protein, 10 aa, synthetic construct
RKLKKKIKKL

SEQ ID NO: 77 - protein, 10 aa, synthetic construct
REKKKFLKRR

SEQ ID NO: 78 - protein, 20 aa, synthetic construct
KRKGDEVDGVDEVAKKKSKK

SEQ ID NO: 79 - protein, 17 aa, synthetic construct
RKCLQAGMNLEARKTKK

SEQ ID NO: 80 - protein, 1228 aa, unidentified
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 81 - protein, 1307 aa, Acidaminococcus sp. (strain BV3L6)
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 82 - protein, 1300 aa, Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 83 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 84 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 85 - protein, 1259 aa, Moraxella bovoculi
MGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 86 - protein, 1269 aa, Moraxella bovoculi
MGIHGVPAALFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 87 - protein, 1306 aa, Thiomicrospira sp.
MGIHGVPAATKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 88 - protein, 1214 aa, Butyrivibrio sp.
MGIHGVPAAYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 89 - protein, 1296 aa, Brumimicrobium aurantiacum
MKNQINLFTNKFQLSKTLRFELKPQGKTLEHINSKGFIKNDEKRADSYKKMKATIDAFHRDFIDLAMSNVKLTNLIDFEEIYNASNADKKDEKYKTKLSKIQEILRKEIAKGFKGEEVKDIFSKIDKKDLITKLLEEWIIENKIEDIHFDPEFKNFTTYFSGFHQNRKNMYTDQEQSTAIAYRLIHENLPRFIDNINIFQKINKVPDLEENLKKLYQEIEEYLGINAINEAFELEYFNETLSQKGIDIYNLILGGRTAEEGKQKIQGLNEYINLYNQKQDKKNRVPKLKVLYKQILSDRTRTSFLPDTFEDDEESSASQKVLDSINNFYLENLIDYLPNDKNSTINVLENLKLLLAELINFELDKVYIKNDTSITNISMKIFKNYSVIREALNYFYENKIDPNFAHNFNNANTDKKREKLEKEKAKITKQTYLSISFIEEAIHLYINENSNGNQYKNTYKPNCIANYFKDFFIAENKEGSNKEFDFISKIKARYNTIKGVLNTPFPDNKRLHQEKNNIDNIKHFLDSIMEYLHFAKPLVLSGSFAFEKDEQFYTNFDELYNQLELIIPLYNKVRNYATQKPYSTEKFKLNFENSTLLNGWDVNKEEANTSILFIKNGFYYLGIMDKNHNKIFRNTPKSTNTDIYKKVNYKLLPGASKMLPKVFFGKKNLDYYKPSKDILRIRNHGTHTKGGKPQSGFDKLDFNLNDCHKLIDFFKDSIQKHPDWSKFKFKFSDTQIYESIDQFYRELEPQAYSITYTNIDSSFIEEQINEGKLYLFQIYNKDFSKFSNGKPNLHTLYWKALFDEQNLKDVTYKLNGEAEIFYRKKSIQHDRQIIHKRNQPIINKNPNNEKKESIFKYNIIKDKRYTIDKFQFHVPITLNFKAKGTDYINYDVLDYLKENPDVKIIGLDRGERHLIYLTLIDQKGKILEQISLNEIVNKKHNITTSYHNLLETKEIERDKARKNWGTVETIKELKEGYISQVVHKISKMMIEHNAIVVMEDLNMGFKRGRFKVEKQVYQKLEKMLIDKLNYLVLKDRQPNEPAGIYNALQLTNKFESFQKLGKQSGFLFYVPAWNTSKIDPTTGFVNLFHVKYESVRKSQEFFNKFNSIKYNPKEAIFEFDFDYNEFTTRAEGTKTNWTVCTYGDRIKTFRNPEKLNQWDNKEINITTAFEDFFGRHNITYGNGSDIKSQLISREEKDFFSELIHLFRLTLQMRNSKTNSEIDYLISPVKNENGFFYDSRHADKNLPKDADANGAYHIAKKGLQWIKEIQSFEGNEWKKLKLDKTNKGWLKFVQENQ

SEQ ID NO: 90 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 91 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVISTAVLEYITQQVAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEHNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 92 - protein, 1259 aa, Eubacterium ventriosum
MESNNKIFTETIGTSSIAKTMRNSLVPTESTKRNIEKNGIIIDDQLRAEKRQQLKEIMDEYYRAYIDSKLSNVALTRTIDWKELFQAIENNYKQNTTKTKNELEKKQKEKRTEIYKILSDDEEFKQLFNAKLLTNILPEFIKNQNIDNEEKQEKISTVELFQRFTSSFTDFFKNRKNVFSKDEISTSICYRVVQENAWIFYQNLLAFEEIKKTAEQEIKKIEAENRDSISDYSLKEIFDFDFYGLLLNQGGIRFYNDVCGKINYHMNLYGQKHNIKSNKFKMKRMHKQILSIDESTFEVPTMFENDKEVYQVLNEFLSDLASKKILERVEKIGENVSEYEINKIYIQSKNFENFSSFMCGNWQIINDSLKTYYNEKIKSKGKAKVEKVKKAIKAIEYKSLADINQLVERYNHDELNRKAEEYISAINEKIKDLYVNEIEFDEKTNLIENETKSEEIKSKLDSIMEIMHWTKMFIIEEEIEKDVNFYNEIEEIYDELQPLVTIYNRIRNYVTQKPYSEEKIKLNFGIPTLANGWSKTKEYDNNAIIMIRDGKYYLGIFNAKNKPDKKIMEGHQSEENGDYKKMIYRLLPGPNKMLPKVFMSKTGIAEYKPSQYILECYEQNKHIKSDKNFDIKFCRDLIDFFKTSINRHPEWSKFNFKFSETSEYEDISTFYREVEKQGYKIEWTYISEKEIKELDENGQLYLFQIYNKDFSEKSKGKENLHTMYLKNLFSEENLKNIVLKLNGEAEVFFRKSSIKKPIIHKKGSVLVNKTYNENGERKSIPEEQYTEIYKYLNSIGTNELSEKSKKLMEEGKVEYYKANYDIVKDYRYSVDKFFIHLPMTINFKAAGFSPINNIALKSIALKEDMHIIGIDRGERNLIYVSVIDTKGNIVEQRNFNIVNGIDYKEKLKQKELDRDNARKNWKEIGKIKDLKEGYLSLVVHEIAKLVVKYNAIITMEDLNQGFKRGRFKVERQVYQKFETMLINKLNYLVDKDLAVDQEGGLLRGYQLTYIPESLKVLGRQCGYIFYVPAAYTSKIDPTTGFVAIFNYKGMTDKDFVTSFDSIKYDDERGLFAFEFDYENFVTHKVEMARNKWTVYTYGERIKRKFKNGLWDTAEKVDLTYQMRSILEKYEIEYNKGQDILEQIEELDEKAQNGICKEIKYLVKDIVQMRNSLPDNAVEDYDAIISPVINNNGEFFDSTRGDEDKPLDADANGAYCIALKGLYEVMQIKKNWNEETEFPRKELKIRHQDWFDFIQNKRYL

SEQ ID NO: 93 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 94 - protein, 1182 aa, Leptotrichia wadei
MYMKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKFEILENEFLENVKAMLEYSE

SEQ ID NO: 95 - protein, 1385 aa, Leptotrichia sp.
MGNLFGHKRWYEVRDKKDFKIKRKVKVKRNYDGNKYILNINENNNKEKIDNNKFIGEFVNYKKNNNVLKEFKRKFHAGNILFKLKGKEEIIRIENNDDFLETEEVVLYIEVYGKSEKLKALEITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDNKIDVILTNFMEIREKIKSNLEIMGFVKFYLNVSGDKKKSENKKMFVEKILNTNVDLTVEDIVDFIVKELKFWNITKRIEKVKKFNNEFLENRRNRTYIKSYVLLDKHEKFKIERENKKDKIVKFFVENIKNNSIKEKIEKILAEFKINELIKKLEKELKKGNCDTEIFGIFKKHYKVNFDSKKFSNKSDEEKELYKIIYRYLKGRIEKILVNEQKVRLKKMEKIEIEKILNESILSEKILKRVKQYTLEHIMYLGKLRHNDIVKMTVNTDDFSRLHAKEELDLELITFFASTNMELNKIFNGKEKVTDFFGFNLNGQKITLKEKVPSFKLNILKKLNFINNENNIDEKLSHFYSFQKEGYLLRNKILHNSYGNIQETKNLKGEYENVEKLIKELKVSDEEISKSLSLDVIFEGKVDIINKINSLKIGEYKDKKYLPSFSKIVLEITRKFREINKDKLFDIESEKIILNAVKYVNKILYEKITSNEENEFLKTLPDKLVKKSNNKKENKNLLSIEEYYKNAQVSSSKGDKKAIKKYQNKVTNAYLEYLENTFTEIIDFSKFNLNYDEIKTKIEERKDNKSKIIIDSISTNINITNDIEYIISIFALLNSNTYINKIRNRFFATSVWLEKQNGTKEYDYENIISILDEVLLINLLRENNITDILDLKNAIIDAKIVENDETYIKNYIFESNEEKLKKRLFCEELVDKEDIRKIFEDENFKFKSFIKKNEIGNFKINFGILSNLECNSEVEAKKIIGKNSKKLESFIQNIIDEYKSNIRTLFSSEFLEKYKEEIDNLVEDTESENKNKFEKIYYPKEHKNELYIYKKNLFLNIGNPNFDKIYGLISKDIKNVDTKILFDDDIKKNKISEIDAILKNLNDKLNGYSNDYKAKYVNKLKENDDFFAKNIQNENYSSFGEFEKDYNKVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFRLIGNNDILERLMKPKKVSVLELESYNSDYIKNLIIELLTKIENTNDTL

SEQ ID NO: 96 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 97 - protein, 1197 aa, Leptotrichia wadei
MKVTKIDGLSHKKFEDEGKLVKFRNNKNINEIKERLKKLKELKLDNYIKNPENVKNKDKDAEKETKIRRTNLKKYFSEIILRKEDEKYILKKTKKFKDINQEIDYYDVKSKKNQQEIFDVLKEILELKIKETEKEEIITFDSEKLKKVFGEDFVKKEAKIKAIEKSLKINKANYKKDSIKIGDDKYSNVKGENKRSRIYEYYKKSENLKKFEENIREAFEKLYTEENIKELYSKIEEILKKTHLKSIVREFYQNEIIGESEFSKKNGDGISILYNQIKDSIKKEENFIEFIENTGNLELKELTKSQIFYKYFLENEELNDENIKFAFCYFVEIEVNNLLKENVYKIKRFNESNKKRIENIFEYGKLKKLIVYKLENKLNNYVRNCGKYNYHMENGDIATSDINMRNRQTEAFLRSIIGVSSFGYFSLRNILGVNDDDFYETEEDLTKKERRNLEKAKEDITIKNTFDEVVVKSFQKKGIYNIKENLKMFYGDSFDNADKDELKQFFVNMLNAITSIRHRVVHYNMNTNSENIFNFSGIEVSKLLKSIFEKETDKRELKLKIFRQLNSAGVFDYWENRKIDKYLENIEFKFVNKNIPFVPSFTKLYNRIDNLKGNNALNLGYINIPKRKEARDSQIYLLKNIYYGEFVEKFVNNNDNFEKIFREIIEINKKDGTNTKTKFYKLEKFETLKANAPIEYLEKLQSLHQINYNREKVEEDKDIYVDFVQKIFLKGFINYLQGSDLLKSLNLLNLKKDEAIANKKSFYDEKLKLWQNNGSNLSKMPEEIYDYIKKIKINKINYSDRMSIFYLLLKLIDHKELTNLRGNLEKYVSMNKNKIYSEELNIVNLVSLDNNKVRANFNLKPEDIGKFLKTETSIRNINQLNNFSEIFADGENVIKHRSFYNIKKYGILDLLEKIVDKADLKITKEEIKKYENLQNELKRNDFYKIQERIHRNYNQKPFLIKNNEKDFNDYKKAIENIQNYTQLKNKIEFNDLNLLQSLLFRILHRLAGYTSLWERDLQFKLKGEYPENKYIDEIFNFDNSKNKIYNEKNERGGSVVSKYGYFLVEKDGEIQRKNARDKKKNKIIKKEGLEIRNYIAHFNYIPDATKSILEILEELRNLLKYDRKLKNAVMKSIKDIFKEYGLIIEFKISHVNNSEKIEVLNVDSEKIKHLKNNGLVTTRNSEDLCELIKMMLEYKKS

SEQ ID NO: 98 - protein, 913 aa, Leptotrichia sp.
MKELIEKVPNVSELKKSQVFYKYYLNKEKLNDENIKYVFCHFVEIEMSKLLKNYVYKKPSNISNDKVKRIFEYQSLKKLIENKLLNKLDTYIRNCGKYSFYLQDGEIATSDFIVGNRQNEAFLRNIIGVSSAAYFSLRNILETENENDITGKMRGKTVKNKKGEEKYISGEIDKLYDNNKQNEVKKNLKMFYSYDFNMNSKKEIEDFFSNIDEAISSIRHGIVHFNLELEGKDIFTFKNIVPSQISKKMFHDEINEKKLKLKIFKQLNSANVFRYLEKYKILNYLNRTRFEFVNKNIPFVPSFTKLYSRIDDLKNSLCIYWKIPKANDNNKTKEITDAQIYLLKNIYYSEFLNYFMSNNGNFFEIIKEIIELNKNDKRNLKTGFYKLQKFENLQEKTPKEYLANIQSFYMIDAGNKDEEEKDAYIDFIQKIFLKGFMTYLANNGRLSLMYIGNDEQINTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKISDEAKYKISIEELKNYSNKKNEIEKNHTNQENLHRKYARPRKDEKFNDEDYKKYEKAIRNIQQYTHLKNKVEFNELNLLQSLLLRILHRLVGYTSIWERDLRFRLKGEFPENQYIEEIFNFNNSKNVKYKNGQIVEKYISFYKELYKDDTEKISIYSDKKVKELKKEKKDLYIRNYIAHFNYIPNAEISLLEVLENLRKLLSYDRKLKNAIMKSIVDILKEYGFVVTFKIEKDKKIRIESLKSEEVVHLKKLKLKDNDKKKEPIKTYRNSKELCELVKVMFEYKMKEKKSEN

SEQ ID NO: 99 - protein, 1437 aa, unidentified
MKISKVREENRGAKLTVNAKTAVVSENRSQEGILYNDPSRYGKSRKNDEDRDRYIESRLKSSGKLYRIFNEDKNKRETDELQWFLSEIVKKINRRNGLVLSDMLSVDDRAFEKAFEKYAELSYTNRRNKVSGSPAFETCGVDAATAERLKGIISETNFINRIKNNIDNKVSEDIIDRIIAKYLKKSLCRERVKRGLKKLLMNAFDLPYSDPDIDVQRDFIDYVLEDFYHVRAKSQVSRSIKNMNMPVQPEGDGKFAITVSKGGTESGNKRSAEKEAFKKFLSDYASLDERVRDDMLRRMRRLVVLYFYGSDDSKLSDVNEKFDVWEDHAARRVDNREFIKLPLENKLANGKTDKDAERIRKNTVKELYRNQNIGCYRQAVKAVEEDNNGRYFDDKMLNMFFIHRIEYGVEKIYANLKQVTEFKARTGYLSEKIWKDLINYISIKYIAMGKAVYNYAMDELNASDKKEIELGKISEEYLSGISSFDYELIKAEEMLQRETAVYVAFAARHLSSQTVELDSENSDFLLLKPKGTMDKNDKNKLASNNILNFLKDKETLRDTILQYFGGHSLWTDFPFDKYLAGGKDDVDFLTDLKDVIYSMRNDSFHYATENHNNGKWNKELISAMFEHETERMTVVMKDKFYSNNLPMFYKNDDLKKLLIDLYKDNVERASQVPSFNKVFVRKNFPALVRDKDNLGIELDLKADADKGENELKFYNALYYMFKEIYYNAFLNDKNVRERFITKATKVADNYDRNKERNLKDRIKSAGSDEKKKLREQLQNYIAENDFGQRIKNIVQVNPDYTLAQICQLIMTEYNQQNNGCMQKKSAARKDINKDSYQHYKMLLLVNLRKAFLEFIKENYAFVLKPYKHDLCDKADFVPDFAKYVKPYAGLISRVAGSSELQKWYIVSRFLSPAQANHMLGFLHSYKQYVWDIYRRASETGTEINHSIAEDKIAGVDITDVDAVIDLSVKLCGTISSEISDYFKDDEVYAEYISSYLDFEYDGGNYKDSLNRFCNSDAVNDQKVALYYDGEHPKLNRNIILSKLYGERRFLEKITDRVSRSDIVEYYKLKKETSQYQTKGIFDSEDEQKNIKKFQEMKNIVEFRDLMDYSEIADELQGQLINWIYLRERDLMNFQLGYHYACLNNDSNKQATYVTLDYQGKKNRKINGAILYQICAMYINGLPLYYVDKDSSEWTVSDGKESTGAKIGEFYRYAKSFENTSDCYASGLEIFENISEHDNITELRNYIEHFRYYSSFDRSFLGIYSEVFDRFFTYDLKYRKNVPTILYNILLQHFVNVRFEFVSGKKMIGIDKKDRKIAKEKECARITIREKNGVYSEQFTYKLKNGTVYVDARDKRYLQSIIRLLFYPEKVNMDEMIEVKEKKKPSDNNTGKGYSKRDRQQDRKEYDKYKEKKKKEGNFLSGMGGNINWDEINAQLKN

SEQ ID NO: 100 - protein, 1334 aa, unidentified
MKISKVDHTRMAVAKGNQHRRDEISGILYKDPTKTGSIDFDERFKKLNCSAKILYHVFNGIAEGSNKYKNIVDKVNNNLDRVLFTGKSYDRKSIIDIDTVLRNVEKINAFDRISTEEREQIIDDLLEIQLRKGLRKGKAGLREVLLIGAGVIVRTDKKQEIADFLEILDEDFNKTNQAKNIKLSIENQGLVVSPVSRGEERIFDVSGAQKGKSSKKAQEKEALSAFLLDYADLDKNVRFEYLRKIRRLINLYFYVKNDDVMSLTEIPAEVNLEKDFDIWRDHEQRKEENGDFVGCPDILLADRDVKKSNSKQVKIAERQLRESIREKNIKRYRFSIKTIEKDDGTYFFANKQISVFWIHRIENAVERILGSINDKKLYRLRLGYLGEKVWKDILNFLSIKYIAVGKAVFNFAMDDLQEKDRDIEPGKISENAVNGLTSFDYEQIKADEMLQREVAVNVAFAANNLARVTVDIPQNGEKEDILLWNKSDIKKYKKNSKKGILKSILQFFGGASTWNMKMFEIAYHDQPGDYEENYLYDIIQIIYSLRNKSFHFKTYDHGDKNWNRELIGKMIEHDAERVISVEREKFHSNNLPMFYKDADLKKILDLLYSDYAGRASQVPAFNTVLVRKNFPEFLRKDMGYKVHFNNPEVENQWHSAVYYLYKEIYYNLFLRDKEVKNLFYTSLKNIRSEVSDKKQKLASDDFASRCEEIEDRSLPEICQIIMTEYNAQNFGNRKVKSQRVIEKNKDIFRHYKMLLIKTLAGAFSLYLKQERFAFIGKATPIPYETTDVKNFLPEWKSGMYASFVEEIKNNLDLQEWYIVGRFLNGRMLNQLAGSLRSYIQYAEDIERRAAENRNKLFSKPDEKIEACKKAVRVLDLCIKISTRISAEFTDYFDSEDDYADYLEKYLKYQDDAIKELSGSSYAALDHFCNKDDLKFDIYVNAGQKPILQRNIVMAKLFGPDNILSEVMEKVTESAIREYYDYLKKVSGYRVRGKCSTEKEQEDLLKFQRLKNAVEFRDVTEYAEVINELLGQLISWSYLRERDLLYFQLGFHYMCLKNKSFKPAEYVDIRRNNGTIIHNAILYQIVSMYINGLDFYSCDKEGKTLKPIETGKGVGSKIGQFIKYSQYLYNDPSYKLEIYNAGLEVFENIDEHDNITDLRKYVDHFKYYAYGNKMSLLDLYSEFFDRFFTYDMKYQKNVVNVLENILLRHFVIFYPKFGSGKKDVGIRDCKKERAQIEISEQSLTSEDFMFKLDDKAGEEAKKFPARDERYLQTIAKLLYYPNEIEDMNRFMKKGETINKKVQFNRKKKITRKQKNNSSNEVLSSTMGYLFKNIKL

SEQ ID NO: 101 - protein, 1340 aa, unidentified
MQISKVNHKHVAVGQKDRERITGFIYNDPVGDEKSLEDVVAKRANDTKVLFNVFNTKDLYDSQESDKSEKDKEIISKGAKFVAKSFNSAITILKKQNKIYSTLTSQQVIKELKDKFGGARIYDDDIEEALTETLKKSFRKENVRNSIKVLIENAAGIRSSLSKDEEELIQEYFVKQLVEEYTKTKLQKNVVKSIKNQNMVIQPDSDSQVLSLSESRREKQSSAVSSDTLVNCKEKDVLKAFLTDYAVLDEDERNSLLWKLRNLVNLYFYGSESIRDYSYTKEKSVWKEHDEQKANKTLFIDEICHITKIGKNGKEQKVLDYEENRSRCRKQNINYYRSALNYAKNNTSGIFENEDSNHFWIHLIENEVERLYNGIENGEEFKFETGYISEKVWKAVINHLSIKYIALGKAVYNYAMKELSSPGDIEPGKIDDSYINGITSFDYEIIKAEESLQRDISMNVVFATNYLACATVDTDKDFLLFSKEDIRSCTKKDGNLCKNIMQFWGGYSTWKNFCEEYLKDDKDALELLYSLKSMLYSMRNSSFHFSTENVDNGSWDTELIGKLFEEDCNRAARIEKEKFYNNNLHMFYSSSLLEKVLERLYSSHHERASQVPSFNRVFVRKNFPSSLSEQRITPKFTDSKDEQIWQSAVYYLCKEIYYNDFLQSKEAYKLFREGVKNLDKNDINNQKAADSFKQAVVYYGKAIGNATLSQVCQAIMTEYNRQNNDGLKKKSAYAEKQNSNKYKHYPLFLKQVLQSAFWEYLDENKEIYGFISAQIHKSNVEIKAEDFIANYSSQQYKKLVDKVKKTPELQKWYTLGRLINPRQANQFLGSIRNYVQFVKDIQRRAKENGNPIRNYYEVLESDSIIKILEMCTKLNGTTSNDIHDYFRDEDEYAEYISQFVNFGDVHSGAALNAFCNSESEGKKNGIYYDGINPIVNRNWVLCKLYGSPDLISKIISRVNENMIHDFHKQEDLIREYQIKGICSNKKEQQDLRTFQVLKNRVELRDIVEYSEIINELYGQLIKWCYLRERDLMYFQLGFHYLCLNNASSKEADYIKINVDDRNISGAILYQIAAMYINGLPVYYKKDDMYVALKSGKKASDELNSNEQTSKKINYFLKYGNNILGDKKDQLYLAGLELFENVAEHENIIIFRNEIDHFHYFYDRDRSMLDLYSEVFDRFFTYDMKLRKNVVNMLYNILLDHNIVSSFVFETGEKKVGRGDSEVIKPSAKIRLRANNGVSSDVFTYKVGSKDELKIATLPAKNEEFLLNVARLIYYPDMEAVSENMVREGVVKVEKSNDKKGKISRGSNTRSSNQSKYNNKSKNRMNYSMGSIFEKMDLKFD

SEQ ID NO: 102 - protein, 1344 aa, Eubacterium rectale
MLRRDKEVKKLYNVFNQIQVGTKPKKWNNDEKLSPEENERRAQQKNIKMKNYKWREACSKYVESSQRIINDVIFYSYRKAKNKLRYMRKNEDILKKMQEAEKLSKFSGGKLEDFVAYTLRKSLVVSKYDTQEFDSLAAMVVFLECIGKNNISDHEREIVCKLLELIRKDFSKLDPNVKGSQGANIVRSVRNQNMIVQPQGDRFLFPQVYAKENETVTNKNVEKEGLNEFLLNYANLDDEKRAESLRKLRRILDVYFSAPNHYEKDMDITLSDNIEKEKFNVWEKHECGKKETGLFVDIPDVLMEAEAENIKLDAVVEKRERKVLNDRVRKQNIICYRYTRAVVEKYNSNEPLFFENNAINQYWIHHIENAVERILKNCKAGKLFKLRKGYLAEKVWKDAINLISIKYIALGKAVYNFALDDIWKDKKNKELGIVDERIRNGITSFDYEMIKAHENLQRELAVDIAFSVNNLARAVCDMSNLGNKESDFLLWKRNDIADKLKNKDDMASVSAVLQFFGGKSSWDINIFKDAYKGKKKYNYEVRFIDDLRKAIYCARNENFHFKTALVNDEKWNTELFGKIFERETEFCLNVEKDRFYSNNLYMFYQVSELRNMLDHLYSRSVSRAAQVPSYNSVIVRTAFPEYITNVLGYQKPSYDADTLGKWYSACYYLLKEIYYNSFLQSDRALQLFEKSVKTLSWDDKKQQRAVDNFKDHFSDIKSACTSLAQVCQIYMTEYNQQNNQIKKVRSSNDSIFDQPVYQHYKVLLKKAIANAFADYLKNNKDLFGFIGKPFKANEIREIDKEQFLPDWTSRKYEALCIEVSGSQELQKWYIVGKFLNARSLNLMVGSMRSYIQYVTDIKRRAASIGNELHVSVHDVEKVEKWVQVIEVCSLLASRTSNQFEDYFNDKDDYARYLKSYVDFSNVDMPSEYSALVDFSNEEQSDLYVDPKNPKVNRNIVHSKLFAADHILRDIVEPVSKDNIEEFYSQKAEIAYCKIKGKEITAEEQKAVLKYQKLKNRVELRDIVEYGEIINELLGQLINWSFMRERDLLYFQLGFHYDCLRNDSKKPEGYKNIKVDENSIKDAILYQIIGMYVNGVTVYAPEKDGDKLKEQCVKGGVGVKVSAFHRYSKYLGLNEKTLYNAGLEIFEVVAEHEDIINLRNGIDHFKYYLGDYRSMLSIYSEVFDRFFTYDIKYQKNVLNLLQNILLRHNVIVEPILESGFKTIGEQTKPGAKLSIRSIKSDTFQYKVKGGTLITDAKDERYLETIRKILYYAENEEDNLKKSVVVTNADKYEKNKESDDQNKQKEKKNKDNKGKKNEETKSDAEKNNNERLSYNPFANLNFKLSN

SEQ ID NO: 103 - protein, 1228 aa, Lachnospiraceae bacterium; identical to SEQ ID NO: 80

SEQ ID NO: 104 - protein, 1307 aa, Acidaminococcus sp.BV3L6; identical to SEQ ID NO: 81

SEQ ID NO: 105 - protein, 1300 aa, Francisella novicida; identical to SEQ ID NO: 82

SEQ ID NO: 106 - protein, 1246 aa, Porphyromonas macacae; identical to SEQ ID NO: 83

SEQ ID NO: 107 - protein, 1373 aa, Moraxella bovoculi; identical to SEQ ID NO: 84

SEQ ID NO: 108 - protein, 1259 aa, Moraxella bovoculi; identical to SEQ ID NO: 85

SEQ ID NO: 109 - protein, 1269 aa, Moraxella bovoculi; identical to SEQ ID NO: 86

SEQ ID NO: 110 - protein, 1306 aa, Thiomicrospira sp.XS5; identical to SEQ ID NO: 87

SEQ ID NO: 111 - protein, 1214 aa, Btyrivibrio sp.NC3005; identical to SEQ ID NO: 88

SEQ ID NO: 112 - protein, 1129 aa, Unknown
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 113-125 - skipped (13 entries)

SEQ ID NO: 126 - protein, 4 aa, synthetic construct
GGSG

SEQ ID NO: 127 - protein, 5 aa, synthetic construct
GGSGG

SEQ ID NO: 128 - protein, 5 aa, synthetic construct
GSGSG

SEQ ID NO: 129 - protein, 5 aa, synthetic construct
GSGGG

SEQ ID NO: 130 - protein, 5 aa, synthetic construct
GGGSG

SEQ ID NO: 131 - protein, 5 aa, synthetic construct
GSSSG

SEQ ID NO: 132 - protein, 5 aa, synthetic construct
GGGGS

SEQ ID NO: 133 - protein, 5 aa, synthetic construct
GSGGS

SEQ ID NO: 134 - protein, 6 aa, synthetic construct
GGSGGS

SEQ ID NO: 135 - protein, 5 aa, synthetic construct
GGGGS

SEQ ID NO: 136 - protein, 4 aa, synthetic construct
GGGS

SEQ ID NO: 137 - RNA, 589 nt, Woodchuck hepatitis B virus
  source 1..589: mol_type = other RNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 138 - DNA, 267 bp, synthetic construct
  source 1..267: mol_type = other DNA
aattcgattcgtcagtagggttgtaaaggtttttcttttcctgagaaaacaaccttttgttttctcaggttttgctttttggcctttccctagctttaaaaaaaaaaaagcaaaactcaccgaggcagttccataggatggcaagatcctggtattggtctgcgaaatttctactcttgtagatggagacgcaaattctaatccagcttcaacagctatttcacaagctttcatgatttctcgtctctaatttctactcttgtagat

SEQ ID NO: 139 - RNA, 20 nt, synthetic construct
  source 1..20: mol_type = other RNA
taatttctactcttgtagat

(19) United States
(12) Patent Application Publication
(10) Pub. No.: US 2026/0297565 A1
(43) Pub. Date: Oct. 1, 2026

(54) COMPOSITIONS AND METHODS FOR MULTIPLEX CRISPR-CAS SCREENING AND GRNA IDENTIFICATION

(71) Applicant: The Regents of the University of California, Oakland, CA (US)

(72) Inventors: Amy Yi-Tyng Chang, Oakland, CA (US); Rachel Mace, Oakland, CA (US); Adam Litterman, Oakland, CA (US)

(21) Appl. No.: 19/629,745

(22) Filed: Mar. 26, 2026

Related U.S. Application Data
(60) Provisional application No. 63/781,631, filed on Apr. 1, 2025.

(51) Int. Cl. C12N 15/10

(52) U.S. Cl. CPC C12N 15/1065

(57) ABSTRACT

Methods and compositions are provided for performing multiplex CRISPR screens and for identifying CRISPR-Cas guide RNAs produced from gRNA arrays in a single cell. The gRNAs are produced from a DNA molecule of the present disclosure, comprising, e.g., in 5′ to 3′ order, nucleotide sequences encoding a Pol-II promoter, and, operably linked to the Pol II promoter, a gRNA array, a 5′ PCR handle site, and a barcode. In some instances, the 5′ PCR handle site is positioned 5, 10, or 15 nucleotides or more 3′ of the 3′-most direct repeat of the gRNA array. In some cases, the DNA molecule does not include a Pol III promoter. In some embodiments, identifying the gRNAs includes capturing (e.g., using a poly(dT) primer), amplifying (e.g., using a 5′ PCR primer that binds to a 5′ PCR handle), and sequencing a barcode associated with the gRNA array.

### CROSS-REFERENCE

This application claims the benefit of U.S. Provisional Patent Application No. 63/781,631 filed Apr. 1, 2025, which application is incorporated herein by reference in its entirety.

### INCORPORATION BY REFERENCE OF SEQUENCE LISTING PROVIDED AS AN XML FILE

A Sequence Listing is provided herewith as a Sequence Listing XML, “UCSF-852PRV_seq-listing.xml” created on Mar. 31, 2025, and having a size of 192,762 bytes. The contents of the Sequence Listing XML are incorporated by reference herein in their entirety.

### I. INTRODUCTION

In single cell technologies, molecules of interest are captured through sequence complementary oligos, which are designed to introduce both the 5′ and 3′ handles needed for PCR amplification and sequencing library generation. In existing methods, guide RNA capture is achieved in one of two ways. In the first approach, termed CROP-seq (Datlinger et al., 2017), a single Cas9 guide driven by an hU6 promoter is integrated into the 3′ LTR of a lentiviral vector, located downstream of an EF1a promoter driving a puromycin resistance marker. Pol II-driven transcription from the EF1a promoter produces a transcript bearing both the puromycin resistance marker and the hU6 promoter and guide. Notably, this transcript is polyadenylated, facilitating capture of this guide-identifying transcript alongside other polyadenylated mRNAs.

A common strategy for capture of poly(A) transcripts uses primers containing a poly(dT) homopolymer coupled to a defined constant sequence. The poly(dT) homopolymer binds to the poly(A) sitail of transcripts, attaching the defined constant sequence and allowing it to act as a 3′ PCR handle. However, given no analogous constant sequence at the 5′ terminus of poly(A) transcripts, a similar strategy for introducing a 5′ PCR handle is not readily available. To address this challenge, single cell technologies employ a reverse transcriptase that, upon reaching the 5′ terminus of the transcript, appends an untemplated ‘CCC’ to the first strand cDNA product. A template switch oligo (TSO) bearing a ‘GGG’ at the 3′ end is then annealed to the untemplated ‘CCC’, allowing the 5′ defined constant sequence of the TSO to act as a 5′ PCR handle. Once both 5′ and 3′ PCR handles are generated, the molecule undergoes PCR amplification and sequencing library generation. Though this technology has been widely adopted, use of a TSO harbors several drawbacks. Firstly, template switching cannot occur unless the reverse transcriptase reaches the 5′ terminus of the transcript; this leads to length- and sequence-dependent biases in template switching. Additionally, template switching at the 5′ terminus is estimated to be 30% efficient. These combined challenges result in reduced transcript capture and can pose challenges for single cell transcript assignment.

In the second approach, Perturb-seq is a single cell CRISPR technology that captures non-polyadenylated Cas9 guides through platform-specific capture sequences (Replogle et al., 2020). In Perturb-seq, a platform-specific capture sequence is integrated into the guide constant sequence located 3′ to the guide targeting sequence. On the 10× Genomics platform, these are termed Capture Sequence 1 (CS1) and Capture Sequence 2 (CS2). This capture sequence is then captured by CS1-complementary oligos on the 10× single cell platform. In Cas9 Perturb-seq, a CS1 is incorporated into each guide constant region, with each guide transcript containing a CS1 element. Although this strategy has proven powerful for Cas9-based single cell screens, this approach has been deemed unfavorable for Cas12a guide arrays due to commercial oligo synthesis length limitations.

Thus, there is a need for guide capture technologies that allow simultaneous transcriptome and guide array assignment to single cells, and such is provided herein.

### II. SUMMARY

The inventors have discovered a CRISPR-Cas effector protein (e.g., Cas12a) vector architecture that encompasses several advancements in CRISPR-Cas technology, namely the ability to drive uniform transcription of multiplexed guide arrays and the ability to maintain functional expression of selection markers, and the ability to assign transcriptomic profiles and Cas12a guide arrays to single cells. The work described in the experimental examples below led to the surprising finding by the inventors that uniform transcription of multiplexed guide arrays can be achieved by expressing guide arrays under a Pol II promoter rather than the Pol III promoters canonically used for guide expression. Additionally, the inventors have developed a method for capturing a guide RNA array-specific barcode coupled to either a poly(A) tail or a 10× Capture Sequence 1 (CS1) that can be used to identify the gRNA array (and thereby the individual gRNAs of the array) produced in individual cells from, e.g., a CRISPR-Cas effector protein vector, e.g., as provided herein.

In some embodiments, the CRISPR-Cas effector protein vector is a DNA molecule, and the DNA molecule includes, in 5′ to 3′ order, (i) a Pol-II promoter, (ii) the CRISPR-Cas guide RNA (gRNA) array, (iii) a nucleotide sequence encoding the 5′ PCR handle site, and (iv) a nucleotide sequence encoding the barcode; wherein (ii), (iii), and (iv) are operably linked to the Pol-II promoter, and wherein the start of the 5′ PCR handle site is positioned 5 nucleotides or more, 10 nucleotides or more, or 15 nucleotides or more 3′ of the 3′-most direct repeat of the CRISPR-Cas gRNA array. In some embodiments, the DNA molecule does not include a Pol III promoter. In some cases, the DNA molecule includes one or more nucleotide sequences encoding one or more polypeptides (e.g., a selectable marker, e.g., an antibiotic resistance protein, a fluorescent protein) 3′ of the Pol-II promoter and 5′ of the gRNA array. In some instance, the DNA molecule includes a transcript-stabilizing RNA structure (e.g., a MALAT1 triplex sequence) 5′ of the gRNA array.

The methods and compositions of the present disclosure may be used to introduce gene edits and modifications to a cell. As such, in some embodiments, the methods and compositions of the present disclosure include gene editing systems and/or host cells. In some embodiments, a gene editing system includes a CRISPR-Cas effector protein. In some cases, the CRISPR-Cas effector protein is provided as a nucleotide sequence encoding a CRISPR-Cas effector polypeptide. In some instances, a nucleic acid of the present disclosure encodes a CRISPR-Cas effector polypeptide. In some instances, the cell expresses the CRISPR-Cas effector protein. In some embodiments, the CRISPR-Cas effector protein is a Cas12 effector protein (e.g., Cas12a) or a Cas13 effector protein.

In some cases, the compositions and methods provided herein can be used for performing multiplex CRISPR screens. In some embodiments, performing a multiplex CRISPR screen comprises contacting a cell population with a plurality of nucleic acids (e.g., DNA molecules) of the present disclosure. The compositions and methods herein can ensure high expression of the one or more polypeptides while maintaining high expression (e.g., stability) of the guide RNAs encoded by the guide RNA array.

In some embodiments, the compositions and methods provided herein can be used for identifying CRISPR-Cas guide RNAs (gRNAs) produced from a gRNA array in a single cell. As such, provided are methods for capturing a barcoded transcript of a guide RNA array, where the transcript includes a poly(A) tail and/or a 10× Capture Sequence 1 (CS1). Barcode-array associations are identified through deep sequencing of a plasmid library and barcode assignments converted to guide array assignments. Both poly(A) tial and CS1 capturing technologies can produce sufficient barcode capture for single cell guide array assignment, facilitating studies of transcriptome-wide effects of multiplexed genetic perturbations. In some embodiments, a subject method includes capturing a barcoded transcript of a guide RNA array, where the transcript includes a poly(A) tail. As such, in some embodiments, identifying gRNAs of a gRNA array includes capturing transcripts expressed from a DNA molecule as provided herein from a single cell using a poly(dT) as a capture sequence (e.g., where the poly(dT) is coupled to a defined nucleotide sequence that can be used as a 3′ PCR handle), amplifying the barcode using a 5′ PCR primer that binds to the 5′ PCR handle site (and using a 3′ primer that binds to the 3′ PCR handle), and sequencing the barcode.

In some embodiments, the present disclosure provides a host cell. In some cases, a nucleic acid of the present disclosure (e.g., a DNA molecule encoding a gRNA array) is introduced into the host cell. In some instances, a CRISPR-Cas effector protein, or nucleotide sequence encoding said protein, is introduced into the host cell. In some cases, the host cell expresses a CRISPR-Cas effector protein. In some embodiments, the host cell is a mammalian cell. In certain embodiments, the host cell is a human cell.

Reagents, compositions, and kits/systems that find use in practicing the subject methods are provided.

### III. BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 provides a schematic of the guide vector. The mU6-driven cloning site (Pol III) is denoted in purple. The EF1a-driven cloning site (Pol II) is denoted in blue. From 5′ to 3′, these are termed EF1a_1, EF1a_2, EF1a_3, and EF1a_4. The EF1a_4 site located in the 3′ LTR was selected as the preferred guide array cloning site.

FIG. 2 provides a schematic of CD55-targeting guide vectors. Guide vectors were constructed with mU6 driving 1-, 2-, 4-, and 6-guide arrays, and EF1a driving 2-, 4-, and 6-guide arrays out of the EF1a_1, EF1a_2, EF1a_3, or EF1a_4 cloning sites. The 3′ guide of each array is a CD55-targeting guide, while the remaining guides are non-targeting guides.

FIG. 3 illustrates transcriptional repression of CD55 as measured by flow cytometry in K562 cells transduced with guide vector at an MOI of less than 0.3. The % CD55 KD observed in cells with mU6 driving a 1-guide array is denoted by a pink line. % CD55 KD as a function of guide array length and cloning site are displayed.

FIG. 4 provides read counts of individual guides within 6-guide arrays expressed from the EF1a_4 site as determined by small RNA-seq. Reads were aligned to guide sequences and guide read counts normalized by the number of reads within each sample aligning to a snoRNA reference transcriptome.

FIG. 5A-5B provides flow cytometry data of BFP fluorescence (VL1-A) of K562 cells transduced with 6-guide arrays driven out of the (A) mU6 and (B) EF1a_4 cloning sites.

FIG. 6A-6F provides details of the WT, Comp14, and Campa triplex sequences. (A) Alignment of the WT triplex from (Wilusz et al., 2012), the Comp14 triplex from (Wilusz et al., 2012), and the Campa triplex from alignment of two vectors (Campa et al., 2019) published to Addgene: pCE048-SiT-Cas12a (Addgene, #128124) and pCI152-pLenti-SiT-Cas12a (Addgene, #128405). (B) Diagram of WT and Comp14 triplex constructs. The MALAT1-mascRNA element is positioned 3′ to BFP and is separated by ~800 nt and a WPRE element from the Cas12a guide array. (C) Excerpt of the sequence used in Campa et al., Nat Methods 16, 887-893 (2019) (see “Supplementary Note 1” of Campa et al.) The MALAT1 sequence (SEQ ID NO: 1 herein) was followed by a 50 nucleotide (50 nt) sequence (SEQ ID NO: 3) before the first AsCas12a Direct Repeat (as annotated by Campa et al.). (D) Annotated sequence map of the WT triplex vector. AmpR, CMV enhancer, HIV-1 Ψ, LoxP, loxP, MCS (fragment), and ori are present but not shown. (E) Annotated sequence map of the Comp14 triplex vector. AmpR, CMV enhancer, HIV-1 Ψ, loxP, loxP, MCS (fragment), and ori are present but not shown. (F) Annotated sequence map of the Campa triplex vector. HIV-1 Ψ and MCS (fragment) are present but not shown.

FIG. 7A-7E provides flow cytometry data of Cas12a CRISPRi-expressing K562 cells transduced with guide arrays driven out of the (A) mU6, (B) EF1a_4 with no triplex, (C) EF1a_4 with WT triplex, (D) EF1a_4 with Comp14 triplex, and (E) EF1a_4 with Campa triplex cloning sites. (Left) BFP data indicate that the WT and Comp14 triplex constructs partially recover BFP fluorescence. (Right) CD55 KD data indicate that the WT, Comp14, and Campa triplex constructs produce weaker CD55 KD relative to the no triplex construct.

FIG. 8A-8F provides details of the WT_noMASC and Comp14_noMASC triplex sequences. (A) Diagram of WT_noMASC and Comp14_noMASC triplex constructs. The MALAT1 element is positioned 3′ to BFP. The Cas12a guide array is positioned 3′ to the MALAT1 element, with the first guide cleavage site of the array designed to liberate the 3′ of the MALAT1 sequence. (B) Annotated sequence map of the WT_noMASC triplex vector. AmpR, CMV enhancer, HIV-1 Ψ, and loxP are present but not shown. (C) Alignment of WT and WT_noMASC (5′ terminus view). The WT construct harbors an additional ‘TTAA’ after the BFP stop codon, while the WT_noMASC construct harbors an additional ‘T’ after the BFP stop codon. (D) Alignment of WT and WT_noMASC (3′ terminus view). The WT constructs harbors the MASC sequence, ending in ‘TTAGCACTA,’ while the WT-noMASC sequence does not harbor the MASC sequence. The ‘AGCAAAA’ forms the 3′ terminus sequence of the triplex structure, followed directly by the AsCas12a Direct Repeat sequence (highlighted). (E) Annotated excerpt of the WT_noMASC construct used in the present studies. (F) Annotated sequence map of the Comp14_noMASC triplex vector. AmpR, CMV enhancer, HIV-1 Ψ, and LoxP are present but not shown.

FIG. 9A-9D provides flow cytometry data of Cas12a CRISPRi-expressing K562 cells transduced with guide arrays driven out of the (A) mU6, (B) EF1a_4 with no triplex, (C) EF1a_4 with WT_noMASC triplex, (D) and EF1a_4 with Comp14_noMASC triplex cloning sites. (Left) BFP data indicate that the WT_noMASC and Comp14_noMASC triplex constructs partially recover BFP fluorescence. (Right) CD55 KD data indicate that the WT_noMASC and Comp14_noMASC triplex constructs produce equivalent, or greater CD55 KD relative to the no triplex construct.

FIG. 10 demonstrates cell viability of K562 cells transduced with no (blue), mU6 1-guide (red), EF1a_4 with no triplex (green), and EF1a_4 with WT_noMASC triplex (purple) constructs. Measurements were taken at puromycin doses ranging from 0-10 μg/mL across a 7 day time course.

FIG. 11A-B provides diagrams of the Cas12a poly(A)-based guide capture strategy. Primers 1a, 2a, and 3a are positioned 5′ to the guide array and primers 5a, 6a, and 7a are positioned 3′ to the guide array. These primers produce a 5′ PCR handle, while a 3′ PCR handle is produced by binding of the poly(A) sitail binds to the poly(dT) oligo of the 10× bead.

FIG. 12 provides an alignment of sequencing reads produced from Cas12a poly(A)-based guide capture against guide vector map of a 6-guide array with a 3′ CD55-targeting guide driven out of the EF1a_4 site. Of primers positioned 5′ to the guide array (1a, 2a, 3a), primer 2a produced the highest number of sequencing reads. Of primers positioned 3′ to the guide array (5a, 6a, 7a), primer 7a produced the highest number of sequencing reads.

FIG. 13A-C provides example histograms of barcode UMI counts produced with the poly(A)-based Cas12a guide capture strategy with primer 7a. For a given 24 nt barcode, UMI counts per cell were tabulated and plotted as a histogram. Data indicate clear separation of cells with low UMI and high UMI counts of a given barcode, indicating sufficiently high signal-to-noise to allow single cell barcode assignments.

FIG. 14 provides example plots of gene expression levels in cells assigned non-targeting arrays and guide array_201, produced with the poly(A)-based Cas12a guide capture strategy with primer 7a. Single cell assignments produced 834 cells assigned non-targeting guide arrays and 66 cells assigned targeting guide array_201. Gene expression levels of the six genes targeted by guide array_201 were plotted and p-values and avgLFC calculated. Data indicated statistically significant transcriptional repression of CD55, targeted in position 1 of array_201, and B2M, targeted in position 4 of array_201.

FIG. 15A-B provides diagrams of Cas12a CS1-based guide capture strategies. (A) An architecture in which CS1 is at the 3′ terminus of the transcript. (B) An architecture in which an RNA motif is positioned 3′ to CS1. A custom primer at the 5′ priming site produces a 5′ PCR handle. The CS1 element binds to the CS1-complementary oligo of the 10× bead, producing a 3′ PCR handle.

FIG. 16 provides histograms of 8 nt barcode UMI counts produced with the CS1-based Cas12a guide capture strategy. For a given 8 nt barcode, UMI counts per cell were tabulated, plotted as a histogram, and labeled with the corresponding vector architecture. Data indicated that the evopreQ1 and tevopreQ1 architectures resulted in a higher capture rate than CS1-3′. These architectures produced clear separation of cells with low UMI and high UMI counts, indicating sufficiently high signal-to-noise to allow single cell barcode assignments.

FIG. 17 provides a diagram depicting the use of a MALAT1 triplex sequence to stabilize polypeptide expression and increase gRNA production in combination with the Cas12a poly(A)-based guide capture strategy.

FIG. 18A-B shows transcriptional repression of CD55 as measured by flow cytometry in K562 cells transduced with Cas12a machinery at an MOI <0.3 and with guide arrays driven out of the (A) EF1a_4 with no triplex and (B) EF1a_4 with WT_noMASC cloning sites. CD55 KD data indicate that the WT_noMASC triplex construct produces greater CD55 KD relative to the no triplex construct.

FIG. 19 provides read counts of individual guides within 6-guide arrays expressed from the mU6 site as determined by small RNA-seq. Reads were aligned to guide sequences and guide read counts normalized by the number of reads within each sample aligning to a snoRNA reference transcriptome. The sequences of mU6-driven guide arrays A and C are identical to corresponding EF1a4-driven guide arrays.

### IV. DEFINITIONS

The terms “polynucleotide” and “nucleic acid,” used interchangeably herein, refer to a polymeric form of nucleotides of any length, either ribonucleotides or deoxynucleotides or combinations thereof. Thus, this term includes, but is not limited to, single-, double, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA) base pairing with a guide RNA) is considered complementary to both a U and to C. For example, when a G/U base-pair can be made at a given nucleotide position of a protein-binding segment (e.g., dsRNA duplex) of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a guide RNA and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine. Coded amino acids (followed in parentheses by their corresponding three-letter codes and one-letter codes) include: alanine (Ala; A), arginine (Arg; R), asparagine (Asn; N), aspartic acid (Asp; D), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F); proline (Pro; P), serine (Ser; S), threonine (Thr; T), tryptophan (Trp; W), tyrosine (Tyr; Y), or valine (Val; V)

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See J. Mol. Biol. 48:443-453 (1970).

The term “naturally-occurring” or “unmodified” or “wild type” as used herein as applied to a nucleic acid, a polypeptide, a cell, or an organism, refers to a nucleic acid, polypeptide, cell, or organism that is found in nature.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, above). Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may have a naturally occurring amino acid sequence.

“Heterologous,” as used herein, refers to a nucleotide or polypeptide sequence that is not found in the native nucleic acid or protein, respectively. For example, relative to a CRISPR-Cas effector protein of the present disclosure, a heterologous polypeptide comprises an amino acid sequence from a protein other than the CRISPR-Cas effector protein. As another example, a CRISPR-Cas effector protein of the present disclosure can be fused to an active domain from a non-CRISPR-Cas effector protein (e.g., a histone deacetylase), and the sequence of the active domain could be considered a heterologous polypeptide (it is heterologous to the CRISPR-Cas effector protein polypeptide). As another example, a guide sequence of a guide RNA that is heterologous to a protein-binding sequence (a constant region) of a guide RNA is a guide sequence that is not found in nature together with the protein-binding sequence. Likewise, a guide RNA that is not found in nature with a given CRISPR-Cas effector protein can be considered heterologous to the CRISPR-Cas effector protein.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

As used herein, a “promoter sequence” is a DNA regulatory region capable of binding RNA polymerase and initiating transcription of a downstream (3′ direction) coding or non-coding sequence. Eukaryotic promoters will often, but not always, contain “TATA” boxes and “CAT” boxes. Various promoters, including inducible promoters, may be used to drive the various nucleic acids (e.g., vectors) of the present disclosure. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

“Operably linked” refers to a juxtaposition wherein the components so described are in a relationship permitting them to function in their intended manner. For instance, a promoter is operably linked to a coding sequence if the promoter affects its transcription or expression. The coding sequence can also be referred to as operably linked to the promoter.

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression (the coding sequence can also be said to be operably linked to the promoter).

The terms “recombinant expression vector,” or “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “guide RNA” (gRNA) and the like refer to an RNA that guides a CRISPR-Cas effector polypeptide (or a fusion protein comprising a CRISPR-Cas effector polypeptide) to a target sequence in a target nucleic acid.

As used herein, the term “targets” can be used to describe complementarity between nucleic acid molecules. As a non-limiting example, a guide RNA that hybridizes to a particular target sequence within a target nucleic acid (the guide sequence of the guide RNA hybridizes to the target sequence of a target nucleic acid) can be said to “target” that nucleic acid. Likewise, the guide RNA can be said to “target” a particular sequence. For example, a guide RNA that ‘targets’ a particular target sequence has a guide sequence that hybridizes to the target nucleic acid such that the CRISPR-Cas effector protein it is complexed with is ‘guided’ to the target sequence. In other words, a guide RNA can be said to target a particular gene or transcript. For example, if multiple guide RNAs are said to target the same particular gene or transcript, some may target one particular target sequence while others may target a different target sequence. Thus guide RNAs can be referred to as targeting a particular gene or transcript, and can also be referred to as targeting a particular sequence on a gene or transcript.

As used herein, the terms “CRISPR-Cas effector polypeptide” and “CRISPR-Cas effector protein” are used interchangeably and refer to the one or more proteins that bind a guide RNA (forming a ribonucleoprotein complex, i.e., an RNP) in a given a CRISPR-Cas system. CRISPR-Cas systems are subdivided into two major classes. The classes are further divided into types comprising different combinations of proteins. In Class 1 CRISPR-Cas systems, a CRISPR-Cas effector protein comprises multiple subunits, wherein the subunits comprise different Cas proteins. For example, a Class 1 CRISPR-Cas system may be a Type III-A CRISPR-Cas effector polypeptide comprising Cas10/Csm1, Csm2, Csm3, Csm4, and Csm5 polypeptides, each of which is a subunit of the CRISPR-Cas effector polypeptide. In Class 2 CRISPR-Cas systems, a CRISPR-Cas effector protein is a single protein that combines all activities required for interference, such as, e.g., Cas9 as a Type II CRISPR-Cas effector polypeptide or Cas12 as a Type V CRISPR-Cas effector polypeptide. A “CRISPR-Cas effector protein” may also refer to a CRISPR-Cas effector protein that comprises one or more modifications. For example, a CRISPR-Cas effector protein may have one or more amino acid substitutions relative to a corresponding wild-type CRISPR-Cas effector protein or an amino acid sequence from a protein other than the CRISPR-Cas effector protein. In some cases, a CRISPR-Cas effector protein of the present disclosure can be fused to a non-CRISPR-Cas protein. The one or more modifications may also modify the activity of the CRISPR-Cas effector protein relative to the wild type CRISPR-Cas effector protein.

As used herein, the term “perturbation” refers to the effects on one more transcripts, genes, or gene products (including protein) as a result of a mutation or modification of a target sequence. Mutation or modifications include, e.g. small nucleotide insertions or deletions (indels) or a larger deletion, insertion, or inversion. In certain embodiments, the introduction a mutation or modification is referred to as “editing” or “gene editing”.

The term “transformation” is used interchangeably herein with “genetic modification” and refers to a permanent or transient genetic change induced in a cell following introduction of new nucleic acid (e.g., DNA exogenous to the cell) into the cell. Genetic change (“modification”) can be accomplished either by incorporation of the new nucleic acid into the genome of the host cell, or by transient or stable maintenance of the new nucleic acid as an episomal element. Where the cell is a eukaryotic cell, a permanent genetic change is generally achieved by introduction of new DNA into the genome of the cell.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. A stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include viral infection (transduction), transfection, electroporation, particle gun technology, calcium phosphate precipitation, direct microinjection, and the like. The choice of method is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

As used herein, “reverse transcription” refers to the process of copying the nucleotide sequence of an RNA molecule into a DNA molecule. Reverse transcription can be done by contacting an RNA template with an RNA-dependent DNA polymerase, also known as a reverse transcriptase. A reverse transcriptase is a DNA polymerase that transcribes single-stranded RNA into single-stranded DNA. Depending on the polymerase used, the reverse transcriptase can also have RNase H activity for subsequent degradation of the RNA template.

As used herein, “complementary DNA” or “cDNA” can refer to a synthetic DNA reverse transcribed from RNA through the action of a reverse transcriptase. The cDNA may be single-stranded or double-stranded and can include strands that have either or both of a sequence that is substantially identical to a part of the RNA sequence or a complement to a part of the RNA sequence.

As used herein, a “primer” can refer to a short polynucleotide, generally with a free 3′—OH group, that binds to a target or template polynucleotide present in a sample by hybridizing with the target or template, and thereafter promoting extension of the primer to form a polynucleotide complementary to the target or template. For the purposes of this disclosure primers (e.g., for use in PCR reactions, transcription reactions, and the like) include polynucleotides ranging in length from 15 to 100 nucleotides (nt) (e.g., 15-80, 15-70, 15-60, 15-50, 15-40, 15-35, 15-30, 15-25, 15-22, 17-100, 17-80, 17-70, 17-60, 17-50, 17-40, 17-35, 17-30, 17-25, 17-22, 18-100, 18-80, 18-70, 18-60, 18-50, 18-40, 18-35, 18-30, 18-25, 18-22, 19-100, 19-80, 19-70, 19-60, 19-50, 19-40, 19-35, 19-30, 19-25, or 19-22 nt). In some cases a primer has a length of from 18-35 nt (e.g., 18-30, 18-25, 18-22).

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. As such, the articles “a” and “an” are used herein to refer to one or to more than one (i.e., to at least one) of the grammatical object of the article. By way of example, “an element” means one element or more than one element. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, it is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### V. DETAILED DESCRIPTION

As noted above, provided are compositions and methods for guide capture technologies that facilitate simultaneous transcriptome and guide array assignment to single cells. In some embodiments, the methods and compositions include a DNA molecule encoding a CRISPR-Cas guide RNA (gRNA) array, a nucleotide sequence encoding a 5′ PCR handle, and a nucleotide sequence encoding a barcode. In some cases, the gRNA array, the nucleotide sequence encoding the 5′ PCR handle, and the nucleotide sequence encoding a barcode are operably linked to a Pol II promoter. In some instances, the DNA molecule includes, in 5′ to 3′ order, (i) a Pol-II promoter, (ii) the CRISPR-Cas guide RNA (gRNA) array, (iii) a nucleotide sequence encoding the 5′ PCR handle site, and (iv) a nucleotide sequence encoding the barcode; wherein (ii), (iii), and (iv) are operably linked to the Pol-II promoter. In some cases, the start of the 5′ PCR handle site is positioned 5 nucleotides or more 3′ of the 3′-most direct repeat of the CRISPR-Cas gRNA array. In some embodiments, the DNA molecule does not include a Pol III promoter.

In some embodiments, the methods include identifying CRISPR-Cas gRNAs produced in a single cell. In some cases, the methods include expressing a CRISPR-Cas gRNA array transcript from a DNA molecule as provided herein. In some cases, the methods include capturing a transcript of the CRISPR-Cas guide RNA array using a poly(dT) capture sequence (in some cases, the poly(dT) is coupled to a defined nucleotide sequence that can be used as a 3′ PCR handle), amplifying the barcode using a 5′ PCR primer that binds to the 5′ PCR handle site (and using, e.g., a 3′ primer that binds to the 3′ PCR handle), and sequencing the barcode.

In some embodiments, the methods include performing multiplex CRISPR screens. In some cases, the methods include contacting a cell population with a plurality of DNA molecules as provided herein. In some instances, performing multiplex CRISPR screens includes identifying CRISPR-Cas gRNAs produced in a single cell. In embodiments, performing multiplex CRISPR screens includes sequencing a plurality of transcripts from each cell, e.g., sequencing the barcode and/or the transcriptome of each cell.

CRISPR-Cas gRNA Arrays

A nucleic acid molecule (e.g., a natural crRNA) that binds to a CRISPR-Cas effector protein, forming a ribonucleoprotein (RNP) complex, and targets the complex to a specific target sequence within a target nucleic acid is referred to herein as a “guide RNA” (gRNA). Guide RNAs of the present disclosure are capable of binding or otherwise interacting with a subject CRISPR-Cas effector polypeptide to facilitate targeting to a target nucleic acid. A guide RNA can be said to include two segments, a protein-binding segment and a targeting segment.

As will be known to one of ordinary skill in the art, in some embodiments, a guide RNA includes two separate nucleic acid molecules: an “activator” (e.g., tracrRNA) and a “targeter” (e.g., crRNA) and is referred to as a “dual guide RNA”, a “double-molecule guide RNA”, a “two-molecule guide RNA”, or a “dgRNA.” The protein-binding segment (i.e., constant region) is present on the tracrRNA, while the targeting segment (i.e., spacer) is present on the crRNA molecule. In some embodiments, the guide RNA is one molecule (e.g., for some class 2 CRISPR-Cas proteins, the corresponding natural guide RNA is a single molecule; and in some cases, an activator and targeter can be covalently linked to one another, e.g., via intervening nucleotides), and the guide RNA is referred to as a “single guide RNA”, a “single-molecule guide RNA,” a “one-molecule guide RNA”, or simply “sgRNA.”

The protein-binding segment interacts with (binds to) the CRISPR-Cas effector protein. The protein-binding segment of a guide RNA comprises two stretches of nucleotides (the duplex-forming segment of the activator and the duplex-forming segment of the targeter) that are complementary to one another and hybridize to form a double stranded RNA duplex (dsRNA duplex). Thus, the protein-binding segment includes a dsRNA duplex.

Because this region does not need to change each time a new target sequence is selected, this region is also referred to as the “constant region”, “handle”, “repeat”, “direct repeat”, or “scaffold” sequence of the guide RNA. The constant region in some cases is located 5′ of the targeting segment and in some cases located 3′ of the targeting segment (depending on which CRISPR-Cas effector protein is used). A guide RNA can be referred to by the protein to which it corresponds. For example, when a CRISPR-Cas effector protein is a Cas9 protein, the corresponding guide RNA can be referred to as a “Cas9 guide RNA.” Likewise, as another example, when a CRISPR-Cas effector protein is a Cas12a protein, the corresponding guide RNA can be referred to as a “Cas12a guide RNA.”

Scaffold sequences for various CRISPR-Cas guide RNAs are known in the art. For example, in some cases, the portion of the targeter-RNA (e.g., crRNA) that contributes to the scaffold (i.e., is 3′ of the guide sequence) (e.g., when using an S. pyogenes Cas9 protein) includes: 5′-GUUUUAGAGCUAUGCUGUUUUG-3′ (SEQ ID NO: 7). In some cases, it includes: 5′-GUUUUAGAGCUA-3′ (SEQ ID NO: 8). in some cases, the activator-RNA (e.g., tracrRNA) (e.g., when using an S. pyogenes Cas9 protein) includes: ‘5-AAACAGCAUAGCAAGUUAAAAUAAGGCUAGUCCGUUAUCAACUUGAAAAAGUG GCACCGAGUCGGUGCUU-3’ (SEQ ID NO: 9). In some cases (e.g., when using an S. pyogenes Cas9 protein) a sgRNA includes 5′-GUUUUAGAGCUAGAAAUAGCAAGUUAAAAUAAGGCUAGUCCG-3′ (SEQ ID NO: 10). In some cases (e.g., when using an S. pyogenes Cas9 protein) a sgRNA includes 5′-GUUUUAGAGCUAGAAAUAGCAAGUUAAAAUAAGGCUAGUCCGUUAUCAACUUG AAAAAGUGGCACCGAGUCGGUGCUU-3′ (SEQ ID NO: 11). Mutations/variants of the above sequences can also be used and many suitable examples will be known to one of ordinary skill in the art.

Examples of crRNA repeat sequences (also known as the scaffold) for Cas12a proteins include:

LbCas12a crRNA:
(SEQ ID NO: 12)
5′ AAUUUCUACUAAGUGUAGAU 3′-[spacer] 3′
AsCas12a crRNA:
(SEQ ID NO: 13)
5′ AAUUUCUACUCUUGUAGAU 3′-[spacer] 3′
(SEQ ID NO: 139)
5′ UAAUUUCUACUCUUGUAGAU 3′-[spacer] 3′
FnCas12a crRNA:
(SEQ ID NO: 14)
5′ AAUUUCUACUGUUGUAGAU 3′-[spacer] 3′
PmCas12a crRNA:
(SEQ ID NO: 15)
5′ AAUUUCUACUAUUGUAGAU 3′-[spacer] 3′
MbCas12a/Mb2Cas12a/Mb3Cas12a crRNA:
(SEQ ID NO: 16)
5′ AAUUUCUACUGUUUGUAGAU 3′-[spacer] 3′
TsCas 12a crRNA
(SEQ ID NO: 17)
5′ AAUUUCUACUGUUGUAGAU 3′-[spacer] 3′
BsCas12a crRNA
(SEQ ID NO: 18)
5′ AAUUUCUACUAUUGUAGAU 3′-[spacer] 3′

The following sequences are each an example of a scaffold of a naturally existing Cas13a guide RNA (e.g., a scaffold that is 5′ of the guide sequence) (See, e.g., Feng et al., Anal Chem. 2023 Jan. 10; 95(1):206-217):

(SEQ ID NO: 19)
GUAAGAGACUACCUCUAUAUGAAAGAGGACUAAAAC
(Listeria seeligeri) (“Lse”) (LseCas13a)
(SEQ ID NO: 20)
GAUAUAGACCACCCCAAUAUCGAAGGGGACUAAAAC
(Leptotrichia shahii) (“Lsh”) (LshCas13a)
(SEQ ID NO: 21)
AUUUAGACCACCCCAAAAAUGAAGGGGACUAAAAC
(Leptotrichia buccalis) (“Lbu”) (LbuCas13a)
(SEQ ID NO: 22)
GACCACCCCAAAAAUGAAGGGGACUAAAAC
(Leptotrichia buccalis) (“Lbu”) (LbuCas13a)
(SEQ ID NO: 23)
GAUUUAGACUACCCCAAAAACGAAGGGGACUAAAAC
(LwaCas13a)
(SEQ ID NO: 24)
GUCACAACUCCCAUGUAGGCGGAGACUGCAAC
(TccCas13a)
(SEQ ID NO: 25)
GGAUUUAGAGUACCCCAAAAAUGAAGGGGACUAAAAC
(LtrCas13aa)

The targeting segment of a guide RNA provides target specificity to the complex (the RNP complex) by including a targeting segment, which includes a “guide sequence” (also referred to as a “targeting sequence” or a “spacer”), which is a nucleotide sequence that is complementary to (and hybridizes to) a sequence of a target nucleic acid, e.g., a target DNA (and thereby can be said to “target” a specific sequence or “target” a specific gene). A targeter comprises both the guide sequence of the guide RNA and a stretch (a “duplex-forming segment”) of nucleotides that forms one half of the dsRNA duplex of the protein-binding segment of the guide RNA. A corresponding tracrRNA-like molecule (activator) comprises a stretch of nucleotides (a duplex-forming segment) that forms the other half of the dsRNA duplex of the protein-binding segment of the guide RNA. In other words, a stretch of nucleotides of the targeter is complementary to and hybridizes with a stretch of nucleotides of the activator to form the dsRNA duplex of the protein-binding segment of a guide RNA. As such, each targeter can be said to have a corresponding activator (which has a region that hybridizes with the targeter). The targeter molecule additionally provides the guide sequence. Thus, a targeter and an activator (as a corresponding pair) hybridize to form a guide RNA.

The guide sequence can be modified (e.g., by genetic engineering)/designed to hybridize to any desired target sequence within a target nucleic acid and can be changed each time a new target sequence is selected. As would be readily understood by one of ordinary skill in the art, in some CRISPR-Cas systems (e.g., those that target double stranded DNA) the PAM sequence in the target DNA is also taken into consideration when selecting a guide sequence (see, e.g., as further described below). Approaches for designing CRISPR-Cas guide RNAs (also simply referred to herein as “guide RNAs”), and using CRISPR-Cas systems to increase expression or decrease expression of a target gene (e.g., via CRISPRa or CRISPRi, respectively) or to edit a target gene (e.g., induce a mutation in a target gene) are known in the art and any convenient system can be used. For example, when using CRISPRa or CRISPRi to modulate expression of a target gene, a guide RNA is used that hybridizes at or near a transcription start site to inhibit (CRISPRi) or activate (CRISPRa) expression of the target gene. A guide RNA can be said to “target” the gene, and one of ordinary skill in the art would understand how to design an appropriate guide RNA based on the desired outcome—i.e., they would understand what sequences within the target locus to target.

In some cases, the percent complementarity between the guide sequence and the target site of the target nucleic acid is 60% or more (e.g., 65% or more, 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid is 80% or more (e.g., 85% or more, 90% or more, 95% or more, 97% or more, 98% or more, 99% or more, or 100%). In some cases, the percent complementarity between the guide sequence and the target site of the target nucleic acid is 90% or more (e.g., 95% or more, 97% or more, 98% or more, 99% or more, or 100%). In some cases, the percent complementarity between the guide sequence and the target site of the target nucleic acid is 100%.

In some cases, the percent complementarity between the guide sequence and the target site of the target nucleic acid is 60% or more (e.g., 70% or more, 75% or more, 80% or more, 85% or more, 90% or more, 95% or more, 97% or more, 98% or more, 99% or more, or 100%) over 19-25 contiguous nucleotides. In some cases, the percent complementarity between the guide sequence and the target site of the target nucleic acid is 80% or more (e.g., 85% or more, 90% or more, 95% or more, 97% or more, 98% or more, 99% or more, or 100%) over 19-25 contiguous nucleotides. In some cases, the percent complementarity between the guide sequence and the target site of the target nucleic acid is 90% or more (e.g., 95% or more, 97% or more, 98% or more, 99% or more, or 100%) over 19-25 contiguous nucleotides. In some cases, the percent complementarity between the guide sequence and the target site of the target nucleic acid is 100% over 19-25 contiguous nucleotides.

In some cases, the guide sequence has a length in a range of from 17-30 nucleotides (nt) (e.g., 17-25, 17-22, 17-20, 17-18, 19-30, 19-25, 19-22, 19-20, 20-30, 20-25, or 20-22 nt). In some cases, the guide sequence has a length in a range of from 19-25 nucleotides (nt) (e.g., from 19-22, 19-20, 20-25, 20-25, or 20-22 nt). In some cases, the guide sequence has a length of 19 or more nt (e.g., 20 or more, 21 or more, or 22 or more nt; 19 nt, 20 nt, 21 nt, 22 nt, 23 nt, 24 nt, 25 nt, etc.). In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt.

In some cases, the dsRNA duplex region formed between the activator and targeter (i.e., the activator/targeter dsRNA duplex) (e.g., in dual or single guide RNA format) includes a range of from 8-25 base pairs (bp) (e.g., from 8-22, 8-18, 8-15, 8-12, 12-25, 12-22, 12-18, 12-15, 13-25, 13-22, 13-18, 13-15, 14-25, 14-22, 14-18, 14-15, 15-25, 15-22, 15-18, 17-25, 17-22, or 17-18 bp, e.g., 15 bp, 16 bp, 17 bp, 18 bp, 19 bp, 20 bp, 21 bp, etc.). In some cases, the duplex region (e.g., in dual or single guide RNA format) includes 8 or more bp (e.g., 10 or more, 12 or more, 15 or more, or 17 or more bp). In some cases, not all nucleotides of the duplex region are paired, and therefore the duplex forming region can include a bulge.

Thus, in some cases, the duplex-forming segments of the activator and targeter have 70%-100% complementarity (e.g., 75%-100%, 80%-10%, 85%-100%, 90%-100%, 95%-100% complementarity) with one another. In some cases, the duplex-forming segments of the activator and targeter have 70%-100% complementarity (e.g., 75%-100%, 80%-10%, 85%-100%, 90%-100%, 95%-100% complementarity) with one another. In some cases, the duplex-forming segments of the activator and targeter have 85%-100% complementarity (e.g., 90%-100%, 95%-100% complementarity) with one another. In some cases, the duplex-forming segments of the activator and targeter have 70%-95% complementarity (e.g., 75%-95%, 80%-95%, 85%-95%, 90%-95% complementarity) with one another.

In other words, in some cases, the dsRNA duplex formed between the activator and targeter (i.e., the activator/targeter dsRNA duplex) includes two stretches of nucleotides that have 70%-100% complementarity (e.g., 75%-100%, 80%-10%, 85%-100%, 90%-100%, 95%-100% complementarity) with one another. In some cases, the activator/targeter dsRNA duplex includes two stretches of nucleotides that have 85%-100% complementarity (e.g., 90%-100%, 95%-100% complementarity) with one another. In some cases, the activator/targeter dsRNA duplex includes two stretches of nucleotides that have 70%-95% complementarity (e.g., 75%-95%, 80%-95%, 85%-95%, 90%-95% complementarity) with one another.

As would be understood to one of ordinary skill in the art, the guide RNA can be introduced into a cell as an RNA (or as a DNA/RNA hybrid) or can be introduced as a nucleic acid encoding the RNA (e.g., a DNA such as an expression vector such as a viral, plasmid, or minicircle DNA), in which case the cell transcribes the RNA from the introduced DNA. In some cases, the nucleotide sequence encoding the guide RNA is operably linked to a promoter (e.g., a Pol-III promoter such as U6 or H1, a Pol-II promoter such as EF1a). In some cases, one or more guide RNAs (e.g., 1, 2, 3, 4, 5, 6, 1-10, 1-8, 1-6, 1-5, 1-4, 1-3, 2-10, 2-8, 2-6, 2-5, 2-4, 3-10, 3-8, 3-6, 3-5, two or more, three or more, four or more, or five or more) (or nucleotide sequences that encode said guide RNAs) can be introduced into the same cell (e.g., to target different sequences of the same target nucleic, to target different target nucleic acids, etc.).

In some cases, guide RNAs may be provided as a precursor guide RNA array. Precursor guide RNA arrays may also be referred to as “guide RNA arrays”, “gRNA arrays”, “crRNA arrays”, “CRISPR guide RNA arrays”, “guide arrays”, or “CRISPR arrays”. Generally, unless otherwise described, when referring to a guide RNA array, it is intended to mean a DNA molecule having an array, where transcription of the array generates a precursor RNA molecule which is then cleaved into separate guide RNAs. Whether the DNA form, RNA form, or both are referred to will in general be clear from the context. A CRISPR array in the art generally includes (encodes) two or more guide RNAs (e.g., 3 or more, 4 or more, 5 or more, or 6 or more) (e.g., arrayed in tandem as precursor molecules). In other words, in some cases, two or more guide RNAs, each comprising a repeat and a spacer (a scaffold and a guide sequence), can be present on an array (a precursor guide RNA array).

Generally, a guide RNA array starts/begins (at the 5′ end) with a direct repeat (e.g., a scaffold sequence), which is followed by (i.e., in the 3′ direction) the first spacer sequence (guide sequence). Thus, the phrase “the start of the CRISPR-Cas gRNA array is positioned” refers to the positioning of the beginning of the first direct repeat of the guide RNA array.

In some cases, a subject guide RNA array includes (encodes) 2 or more guide RNAs (e.g., 3 or more, 4 or more, 5 or more, 6, or more, 7 or more, 8 or more, 10 or more, 12 or more, 16 or more, 20 or more, 24 or more). In some embodiments of the present disclosure, a subject guide RNA array includes 10 or more guide RNAs (such an array can also be said to include 10 or more spacers or guide sequences) (e.g., 11 or more, 12 or more, 15 or more, 16 or more, 20 or more, or 22 or more). In some cases, a subject guide RNA array includes 16 or more guide RNAs (such an array can also be said to include 16 or more spacers or guide sequences) (e.g., 17 or more, 18 or more, 20 or more, or 22 or more).

In some cases, each guide RNA of a precursor guide RNA array has a different guide sequence. In some cases, two or more guide RNAs of a precursor guide RNA array have the same guide sequence. In some cases, the precursor guide RNA array comprises two or more guide RNAs that target different target sites within the same target nucleic acid molecule. In some cases, the precursor guide RNA array comprises two or more guide RNAs that target different target nucleic acid molecules.

In some embodiments, the guide RNAs of a given array target (i.e., can include guide sequences that hybridize to) different target sites of the same target nucleic acid. For example, the guide RNAs can be tiled across a region of target nucleic acid (e.g., tile across the 3′ UTR of an mRNA, tiled across a coding region of DNA or an mRNA, tiled across a miRNA, and the like). In some embodiments, the guide RNAs of a given array target different target nucleic acid molecules (e.g., different mRNAs, different miRNAs, different DNAs, and the like). Thus, the guide RNAs of a given array can target (i.e., can include guide sequences that hybridize to) different target sites of the same target nucleic acid molecules and/or can target different target nucleic acid molecules (e.g., single nucleotide polymorphisms (SNPs), different strains of a particular virus, etc.). In some cases, each guide RNA of a CRISPR array has a different guide sequence. In some cases, two or more guide RNAs of a precursor guide RNA array have the same guide sequence.

In some embodiments, a CRISPR-Cas effector protein of the present disclosure (e.g., a Cas12 protein such as Cas12a, Cas12b, Cas12c, Cas12d, Cas12e; a Cas13 protein such as Cas 13a, Cas13b, Cas13c, Cas13d; and the like) can cleave a precursor guide RNA (after transcription into RNA) into a mature guide RNA, e.g., by endoribonucleolytic cleavage of the RNA precursor. In some cases, a CRISPR-Cas effector protein of the present disclosure (e.g., a Cas12 protein such as Cas12a, Cas12b, Cas12c, Cas12d, Cas12e; a Cas13 protein such as Cas 13a, Cas13b, Cas13c, Cas13d) can cleave a precursor guide RNA array (that includes more than one guide RNA arrayed in tandem) into two or more individual guide RNAs. In some embodiments, a different protein (e.g., Cas6, RNAseIII, and the like) can cleave a precursor guide RNA into a mature guide RNA, e.g., by endoribonucleolytic cleavage of the precursor. In some cases, a different protein (e.g., Cas6, RNAseIII, and the like) can cleave a precursor guide RNA array (that includes more than one guide RNA arrayed in tandem) into two or more individual guide RNAs. Similarly, in some embodiments, a variant Cas protein (e.g., a variant of Cas12, a variant of Cas13, a variant of Cas6) of the present disclosure can cleave a precursor guide RNA (after it is transcribed into an RNA) into a mature guide RNA. Thus, in some cases, a precursor guide RNA array (e.g., a CRISPR array) can be said to comprise two or more (e.g., 3 or more, 4 or more, 5 or more, 6, or more, 7 or more, 8 or more, 10 or more, 12 or more) guide RNAs (e.g., arrayed in tandem as precursor molecules). In other words, in some cases, two or more guide RNAs, each comprising a repeat and a spacer, can be present on an array (a precursor guide RNA array).

Guide RNAs and CRISPR guide RNA arrays can be produced by any convenient method (e.g., in vitro transcription, chemical synthesis, overlapping PCR, and the like). In some cases, the guide RNAs can be designed to be expressed from an expression vector and/or generated by encoding them on nucleic acid (e.g., an expression vector)—in which case nucleic acids encoding the guide RNAs can be introduced into cells, and the cells produce the guide RNAs via transcription (e.g., using a subject nucleic acid). As such, in some embodiments, a nucleic acid (e.g., an expression vector) encodes a CRISPR array.

In some cases, expression of a CRISPR array or the guide RNAs of the CRISPR array is driven by a Pol III promoter (e.g., U6, H1, and the like). In some cases, expression of a CRISPR array or the guide RNAs of the CRISPR array is driven by a Pol II promoter (e.g., CAG, CMV, SV40, TRE3 GS, EF1a, and the like).

Nucleic Acid Encoding gRNA Array

As described above, the methods and compositions of the present disclosure include a nucleic acid encoding one or more polypeptides and a gRNA array. Generally, a nucleic acid of the present disclosure is a DNA molecule. In some cases, the DNA molecule is provided as a vector. A “vector” or “expression vector” is a replicon, such as plasmid, phage, virus, or cosmid, to which another DNA segment, e.g., an insert of interest such as a sequence encoding a gRNA array, may be attached so as to bring about the replication and/or expression of the attached segment in a cell. An “expression cassette” comprises a DNA sequence (coding or non-coding) operably linked to a promoter.

In some cases, a subject vector is a viral vector (e.g., AAV, lentivirus, adenovirus). Suitable expression vectors include viral expression vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Human Gene Ther 5:1088 1097, 1999; WO 94/12649, WO 93/03769; WO 93/19191; WO 94/28938; WO 95/11984 and WO 95/00655); adeno-associated virus (AAV) (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822 3828; Mendelson et al., Virol. (1988) 166:154 165; and Flotte et al., PNAS (1993) 90:10613 10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, a subject nucleic acid (e.g., recombinant expression vector) of the present disclosure is a recombinant adeno-associated virus (AAV) vector. In some cases, a subject nucleic acid (e.g., recombinant expression vector) of the present disclosure is a recombinant lentivirus vector. In some cases, a recombinant expression vector of the present disclosure is a recombinant retroviral vector. As such, in some cases a nucleic acid is first delivered to virus-producing cells as a plasmid, and the virus that is produced is then used to contact cells in order to deliver the nucleic acid of the disclosure.

In some embodiments, a nucleic acid of the present disclosure includes a nucleotide sequence encoding a gRNA array. As described above, a subject guide RNA array (i.e., CRISPR array) includes 2 or more guide RNAs (e.g., 3 or more, 4 or more, 5 or more, 6, or more, 7 or more, 8 or more, 10 or more, or 12 or more). In some embodiments, the guide RNAs of a given array target (i.e., can include guide sequences that hybridize to) different target sites of the same target nucleic acid. In some embodiments, the guide RNAs of a given array target different target nucleic acid molecules.

In some embodiments, a nucleic acid of the present disclosure includes a nucleotide sequence encoding a polypeptide. In some cases, the polypeptide is a cell marker, e.g., a nucleotide sequence encoding fluorescent protein such as GFP (Green Fluorescent Protein), YFP (Yellow), RFP (Red), or BFP (Blue). In some cases, the fluorescent protein a nuclear localized BFP such as mTagBFP2. Such cell markers can allow visualization of cells that received the nucleic acid (e.g., virus-transduced cells). Cell markers also include, but are not limited to, selectable markers such as antibiotic resistance genes (e.g. puromycin resistance, hygromycin resistance, blasticidine resistance, etc.) (which can encode antibiotic resistance proteins) which facilitate cell selection, e.g., selecting cells to which guide RNAs were delivered by drug treatment. In some embodiments, the polypeptide is BFP. In some embodiments, the polypeptide is a polypeptide encoded by a puromycin resistance gene.

In some embodiments, the polypeptide is a CRISPR-Cas effector polypeptide. CRISPR-Cas effector polypeptides are known in the art and are further described herein, and any suitable CRISPR-Cas effector protein may be used. In some cases, the CRISPR-Cas effector polypeptide is a type V CRISPR-Cas effector polypeptide, e.g., a Cas12a, a Cas12b, a Cas12c, a Cas12d, or a Cas12e polypeptide. In some cases, the CRISPR-Cas effector polypeptide is a type VI CRISPR-Cas effector polypeptide, e.g., a Cas13a polypeptide, a Cas13b polypeptide, a Cas13c polypeptide, or a Cas13d polypeptide. In some cases, the CRISPR-Cas effector polypeptide is a type II CRISPR-Cas effector polypeptide (e.g., a Cas9 protein).

In some embodiments, the nucleic acid includes two or more nucleotide sequences encoding two or more polypeptides (e.g., 3 or more, 4 or more, 5 or more, 6, or more, 7 or more, 8 or more, 10 or more). In such cases, the nucleic acid may further include a nucleotide sequence encoding a self-cleaving 2A peptide (and or an internal ribosome entry sequence (IRES)) positioned between each nucleotide sequence encoding a polypeptide. In some instances, the nucleic acid includes a fluorescent polypeptide and a nucleotide sequence encoding an antibiotic resistance protein. In some embodiments, the nucleic acid includes nucleotide sequences encoding a BFP and an antibiotic resistance protein. In some embodiments, the nucleic acid includes, in 5′ to 3′ order, a nucleotide sequence encoding an antibiotic resistance protein (e.g., a puromycin resistance protein), a self-cleaving 2A peptide, and a fluorescent polypeptide (e.g., a BFP).

In some cases, a nucleic acid of the present disclosure is codon optimized. This type of optimization can entail a mutation of a nucleotide sequence encoding a polypeptide to mimic the codon preferences of the intended host organism or cell while encoding the same protein. Thus, the codons can be changed, but the encoded protein remains unchanged. For example, if the intended target cell was a human cell, a human codon-optimized polypeptide-encoding nucleotide sequence could be used. As another non-limiting example, if the intended host cell were a mouse cell, then a mouse codon-optimized polypeptide-encoding nucleotide sequence could be generated. As another non-limiting example, if the intended host cell were a plant cell, then a plant codon-optimized polypeptide-encoding nucleotide sequence could be generated. As another non-limiting example, if the intended host cell were an insect cell, then an insect codon-optimized polypeptide-encoding nucleotide sequence could be generated. Codon usage tables are readily available, as are computer programs for generating codon optimized sequences.

In some embodiments, the nucleic acid includes a sequence encoding an RNA stabilizing element (i.e., an RNA stabilization motif). In some cases, addition of the RNA stabilizing element increases or stabilizes expression of a polypeptide (e.g., from a coding sequence that is 5′ (upstream) of the RNA stabilizing element) and/or gRNA production (from the guide RNA array) (e.g., position 3′ (downstream) of the RNA stabilizing element). RNA stabilizing structures will be known to one of ordinary skill in the art, and any convenient RNA stabilizing structure can be used. In some embodiments, the RNA stabilizing element is a MALAT1 triplex structure (see, e.g., Wilusz E. J., Baptiste C. K., Lu L. Y. et al. A triple helix stabilizes the 3′ ends of long noncoding RNAs that lack poly(A) tails. Genes Dev 26, 2392-2407 (2012); Campa, C. C., Weisbach, N. R., Santinha, A. J. et al. Multiplexed genome engineering by Cas12a and CRISPR arrays encoded on single transcripts. Nat Methods 16, 887-893 (2019); and US patent application publication No. US20240124873A1; which are incorporated herein by reference). In some embodiments, the MALAT1 triplex structure is encoded by the sequence GATTCGTCAGTAGGGTTGTAAAGGTTTTTCTTTTCCTGAGAAAACAACCTTTTGTT TTCTCAGGTTTTGCTTTTTGGCCTTTCCCTAGCTTTAAAAAAAAAAAAGCAAAA (SEQ ID NO: 1) (also referred to herein as “WT”). In some embodiments, the MALAT1 triplex structure is encoded by the sequence AAAGGTTTTTCTTTTCCTGAGAAATTTCTCAGGTTTTGCTTTTTAAAAAAAAAGCAA AA (SEQ ID NO: 2) (also referred to herein as “Comp14”).

In some cases, the sequence encoding the MALAT 1 triplex structure (i.e., the MALAT1 triplex sequence) does not include a MALAT1-associated small cytoplasmic RNA sequence (mascRNA sequence). In some cases such a mascRNA has the sequence GACGCTGGTGGCTGGCACTCCTGGTTTCCAGGACGGGGTTCAAGTCCCTGCGG TGTCTTTGCTT (SEQ ID NO: 4).

As such, for example, in some cases, the MALAT1 triplex structure is encoded by the sequence of SEQ ID NO: 1 and does not include SEQ ID NO: 4; in such cases the MALAT1 triplex can be referred to as “WT_noMASC”. As another example, in some cases, the MALAT1 triplex structure is encoded by the sequence of SEQ ID NO: 2 and does not include SEQ ID NO: 4; in such cases the MALAT1 triplex can be referred to as “Comp14_noMASC”.

In some cases, the MALAT1 triplex structure includes a sequence that 90% or more identical (e.g., 93% or more, 95% or more, 97% or more, 98% or more, 99% or more, or 99.5% or more) to SEQ ID NO: 1. In some cases, the MALAT1 triplex structure includes a sequence that 95% or more identical (e.g., 97% or more, 98% or more, 99% or more, or 99.5% or more) to SEQ ID NO: 1. In some cases, the MALAT1 triplex structure includes a sequence that 98% or more identical (e.g., 99% or more, or 99.5% or more) to SEQ ID NO: 1.

In some cases, the MALAT1 triplex structure includes a sequence that 90% or more identical (e.g., 93% or more, 95% or more, 97% or more, 98% or more, 99% or more, or 99.5% or more) to SEQ ID NO: 1 and does not include mascRNA (e.g., does not include SEQ ID NO: 4). In some cases, the MALAT1 triplex structure includes a sequence that 95% or more identical (e.g., 97% or more, 98% or more, 99% or more, or 99.5% or more) to SEQ ID NO: 1 and does not include mascRNA (e.g., does not include SEQ ID NO: 4). In some cases, the MALAT1 triplex structure includes a sequence that 98% or more identical (e.g., 99% or more, or 99.5% or more) to SEQ ID NO: 1 and does not include mascRNA (e.g., does not include SEQ ID NO: 4).

In some cases, the MALAT1 triplex structure includes a sequence that 90% or more identical (e.g., 93% or more, 95% or more, 97% or more, 98% or more, 99% or more, or 99.5% or more) to SEQ ID NO: 2. In some cases, the MALAT1 triplex structure includes a sequence that 95% or more identical (e.g., 97% or more, 98% or more, 99% or more, or 99.5% or more) to SEQ ID NO: 2. In some cases, the MALAT1 triplex structure includes a sequence that 98% or more identical (e.g., 99% or more, or 99.5% or more) to SEQ ID NO: 2.

In some cases, the MALAT1 triplex structure includes a sequence that 90% or more identical (e.g., 93% or more, 95% or more, 97% or more, 98% or more, 99% or more, or 99.5% or more) to SEQ ID NO: 2 and does not include mascRNA (e.g., does not include SEQ ID NO: 4). In some cases, the MALAT1 triplex structure includes a sequence that 95% or more identical (e.g., 97% or more, 98% or more, 99% or more, or 99.5% or more) to SEQ ID NO: 2 and does not include mascRNA (e.g., does not include SEQ ID NO: 4). In some cases, the MALAT1 triplex structure includes a sequence that 98% or more identical (e.g., 99% or more, or 99.5% or more) to SEQ ID NO: 2 and does not include mascRNA (e.g., does not include SEQ ID NO: 4).

In some embodiments, a MALAT1 triplex sequence in a nucleic acid of the present disclosure is positioned 3′ of the one or more nucleotide sequences encoding one or more polypeptides and 5′ of the nucleotide sequences encoding the gRNA array. In other words, the nucleic acid of the present disclosure includes, in 5′ to 3′ order, the polypeptide-encoding sequence(s), the MALAT1 triplex sequence, and the gRNA array sequence. In some cases, the start of the gRNA array sequence is positioned within 40 nucleotides of the end of the MALAT1 triplex sequence (e.g., the gRNA array sequence is 40 nucleotides or less, 35 nucleotides or less, 32 nucleotides or less, 30 nucleotides or less, 28 nucleotides or less, 25 nucleotides or less, 22 nucleotides or less, 20 nucleotides or less, 18 nucleotides or less, 15 nucleotides or less, 12 nucleotides or less, 10 nucleotides or less, 8 nucleotides or less, 6 nucleotides or less, 4 nucleotides or less, 3 nucleotides or less, 2 nucleotides or less, or 1 nucleotide or less) from the end of the MALAT1 triplex sequence. In some cases, the start of the gRNA array sequence is positioned within 10 nucleotides (e.g., 5 nucleotides or less) of the end of the MALAT1 triplex sequence. In some embodiments, the start of the gRNA array sequence is immediately adjacent to the end of the MALAT1 triplex sequence. In some embodiments, the nucleic acid of the present disclosure does not include the 50 nt sequence CTCACCGAGGCAGTTCCATAGGATGGCAAGATCCTGGTATTGGTCTGCGA (SEQ ID NO: 3) between the end of the MALAT1 triplex sequence and the start of the gRNA array sequence. In some cases, the nucleic acid includes a nucleotide sequence encoding a regulatory element (e.g., a Woodchuck Hepatitis Virus (WHV) Posttranscriptional Regulatory Element (WPRE), encoded by e.g., SEQ ID NO: 137, and as further described below) positioned between the MALAT1 triplex sequence and the gRNA array sequence.

In some embodiments, the nucleic acid of the present disclosure includes one or more RNA stabilization motifs positioned 3′ of the gRNA array. In some cases, the one or more RNA stabilization motifs is positioned 3′ of a reverse transcription handle (e.g., CS1, CS2). In some instances, the one or more RNA stabilization motifs is positioned at the 3′ terminus of the nucleic acid. Stably structured RNA elements are known in the art (see, e.g., Mendez-Mancilla, A. et al. Cell Chem. Biol. 1-7 (2021), which are incorporated herein by reference). In certain embodiments, the RNA stabilization motif is a MALAT1-triplex structure (see Brown, J. A. et al. Proc. Natl. Acad. Sci. U.S.A 109, 19202-19207 (2012), which is incorporated herein by reference). In certain embodiments, the RNA stabilization motif is a NEAT1 (MENB) structure (see Brown, J. A. et al. Proc. Natl. Acad. Sci. U.S.A 109, 19202-19207 (2012), which is incorporated herein by reference). In certain embodiments, the RNA stabilization motif is a spnpreQ1 structure (see Kang, M. et al. Proc. Natl. Acad. Sci. U.S.A. 111, (2014), which is incorporated herein by reference). In certain embodiments, the RNA stabilization motif is an mpknot element (see Anzalone, A. V. et al. Nat. Methods 13, 453-458 (2016), which is incorporated herein by reference). In certain embodiments, the RNA stabilization motif is a ZIKA virus-derived xrRNA1 dumbbell (see Akiyama, B. M. et al. Science. 354, 1148-1152 (2016), which is incorporated herein by reference). In certain embodiments, the RNA stabilization motif is a evopreQ1 pseudoknot (see, e.g., Nelson, J. W. et al. Nat. Biotechnol. (2021) doi: 10.1038/s41587-021-01039-7).

In some embodiments, the nucleic acid of the present disclosure includes regulatory elements that control expression of one or more nucleotide sequences of the nucleic acid. As would be understood to one of ordinary skill in the art, any of a number of suitable transcription and translation control elements, including constitutive and inducible promoters, transcription enhancer elements, transcription terminators, etc. may be used in the nucleic acid. In some cases, the nucleotide sequence(s) encoding the component(s) (e.g., one or more polypeptides, a gRNA array, a 5′ PCR handle, a barcode) of the nucleic acid of the present disclosure is operably linked to a control element, e.g., a transcriptional control element, such as a promoter. In cases where the nucleic acid includes multiple components (e.g., one or more polypeptides, a gRNA array, a 5′ PCR handle, a barcode), the nucleotide sequences encoding the components may be operably linked to a single promoter. In some cases, the nucleotide sequences encoding the components are operably linked to two or more different promoters. Thus, e.g., in some cases, nucleotide sequences encoding the components are each operably linked to a different promoter.

The transcriptional control element can be a promoter. A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Promoters can be used to drive expression by any RNA polymerase (e.g., Pol I, Pol II, Pol III). Exemplary promoters include, but are not limited to the SV40 early or late promoter, retrovirus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31 (17)), a human H1 promoter (H1), mouse metallothionein-I, and the like. Selection of the appropriate vector and promoter is well within the level of ordinary skill in the art.

In some embodiments, the promoter is a Pol II promoter. One skilled in the art would recognize appropriate Pol II promoters for use in the methods and compositions of the present disclosure. Examples of Pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1a (i.e., EF1α) promoter. In some cases, the Pol II promoter is EF1a. In some embodiments, the nucleic acid of the present disclosure includes, in 5′ to 3′ order, a Pol II promoter, one or more polypeptide sequence(s), the MALAT1 triplex sequence, and the gRNA array sequence, such that the polypeptide sequence(s), the MALAT1 triplex sequence, and the gRNA array sequence are operably linked to the Pol II promoter. In some embodiments, the nucleic acid of the present disclosure includes, in 5′ to 3′ order, a Pol-II promoter, a gRNA array sequence, a nucleotide sequence encoding the 5′ PCR handle site, and a nucleotide sequence encoding the barcode, such that the gRNA array sequence, the nucleotide sequence encoding the 5′ PCR handle site, and the nucleotide sequence encoding the barcode are operably linked to the Pol II promoter. In some embodiments, a nucleic acid of the present disclosure does not include a Pol III promoter (e.g., U6, U3, H1, and 7 SL).

Regulatory elements comprise but are not limited to: promoter; enhancer; transcription factor; transcription terminator; efficient RNA processing signals such as splicing and polyadenylation signals (polyA); sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. Also, see Goeddel; Gene Expression Technology: Methods in Enzymology 185, Academic Press, San Diego, CA (1990). Regulatory sequences include those which direct constitutive expression of a nucleic acid sequence in many types of target cell and those which direct expression of the nucleic acid sequence only in certain target cells (e.g., tissue-specific regulatory sequences). Furthermore, a nucleic acid of the present disclosure may include a regulatory sequence to direct synthesis of its components (e.g., one or more polypeptides, a MALAT1 triplex structure, a gRNA array) at specific intervals, or over a specific time period. It will be appreciated by those skilled in the art that the design of the vector can depend on such factors as the choice of the target cell, the level of expression desired, and the like.

In some embodiments, a nucleic acid of the present disclosure has one or more modifications, e.g., a base modification or substitution, a backbone modification, a modified internucleoside linkage, a nucleic acid mimetic, a modified sugar moiety, a conjugate moiety, etc., to provide the nucleic acid with a new or enhanced feature (e.g., improved stability). Nucleic acid modifications are well known and may be readily selected by those skilled in the art (e.g., see international patent application publication No. WO2024112479).

In some instances, the present disclosure provides a plurality of nucleic acids each encoding a gRNA array. In some cases, each nucleic acid includes a different gRNA array, i.e., the gRNA array of each nucleic acid includes one or more different gRNAs than the gRNA array of another nucleic acid of the plurality. In some embodiments, a library of CRISPR-Cas gRNA arrays is provided which includes multiple different arrays.

In embodiments of the present disclosure that include a plurality of nucleic acids encoding different gRNA arrays, it may be desirable to determine which gRNA array is associated with a particular nucleic acid and/or identify the gRNAs within the array. For example, if a plurality of cells is contacted with a plurality of nucleic acids of the present disclosure, it may be desirable to identify the gRNA array and/or the specific gRNAs that were introduced into an individual cell. As such, in some embodiments, a nucleic acid of the present disclosure includes barcode sequences and/or sequences that facilitate capturing and identifying nucleic acid sequences.

As such, in some embodiments, a nucleic acid of the present disclosure includes a 5′ PCR handle site (i.e., a 5′ PCR primer binding site) (also referred to as a “5′ PCR handle”). The term “PCR handle site” (also referred to as a “PCR handle”) refers to a primer binding site capable of binding to a primer to facilitate PCR. The PCR handle has a length of 15-100 nucleotides (nt) (e.g., 15-80, 15-70, 15-60, 15-50, 15-40, 15-35, 15-30, 15-25, 15-22, 17-100, 17-80, 17-70, 17-60, 17-50, 17-40, 17-35, 17-30, 17-25, 17-22, 18-100, 18-80, 18-70, 18-60, 18-50, 18-40, 18-35, 18-30, 18-25, 18-22, 19-100, 19-80, 19-70, 19-60, 19-50, 19-40, 19-35, 19-30, 19-25, or 19-22 nt), or a length within a range of any two of the foregoing lengths. In some cases, the PCR handle has a length of from 18-35 nt (e.g., 18-30, 18-25, 18-22), The design and selection of appropriate PCR primers and primer binding sites are known in the art and routine, and any convenient primers and primer binding sites can be used.

By including a 5′ PCR handle site, the nucleic acid, or a portion thereof, or a cDNA thereof, can be amplified using a 5′ PCR primer (and, e.g., a 3′ primer that binds to a 3′ PCR handle, e.g., provide by the capturing nucleic acid) and sequenced, thereby identifying the nucleotide sequences of the nucleic acid. In some cases, the 5′ PCR handle site is located 5′ of the gRNA array, and the identity of a gRNA array and/or specific gRNAs in the array can be determined by sequencing at least the portion of the nucleic acid or cDNA thereof containing the nucleotide sequence encoding the gRNA array. In some cases, the 5′ PCR handle site is located 3′ of the gRNA array.

In certain embodiments, the 5′ PCR handle site is a primer binding site to initiate extension in a PCR of a complementary strand that includes a barcode sequence. In some embodiments, a nucleic acid of the present disclosure includes a barcode sequence. A barcode is a region that can have a variable base composition or sequence as compared to other labelled nucleic acids in the plurality. As such, each barcoded (i.e., labelled) nucleic acid is associated with a unique nucleotide sequence. The labelled nucleic acid can thereby be identified by sequencing the barcode (e.g., one can extrapolate presence of a given gRNA array based on identification of the associated barcode).

In some instances, a barcode of a nucleic acid of the present disclosure identifies the gRNA array of said nucleic acid, i.e., each barcode is associated with a specific gRNA array, such that sequencing the barcode identifies the gRNA array, which reveals the specific gRNAs in the array. In other words, the barcode, or barcode sequence, is a unique DNA sequence that corresponds to a specific array, such that the barcode encodes the collective identity of the perturbations included in the array. In some embodiments, a nucleic acid of the present disclosure includes, in 5′ to 3′ order, a 5′ PCR handle site and a barcode. By including a 5′ PCR handle site 5′ of a barcode, the portion of the nucleic acid, or cDNA thereof, containing the barcode can be amplified using a 5′ PCR primer and sequenced, thereby identifying the gRNA array and/or specific gRNAs in the array associated with said barcode. In some cases, the 5′ PCR handle site and barcode are positioned 3′ of the nucleotide sequence encoding the gRNA array. In some embodiments, the 5′ PCR handle site and barcode are 5 nucleotides or more 3′ of the 3′-most direct repeat of the gRNA array, such as, e.g., 6 nucleotides or more, 7 nucleotides or more, 8 nucleotides or more, 9 nucleotides or more, 10 nucleotides or more, 12 nucleotides or more, 14 nucleotides or more, 15 nucleotides or more, 18 nucleotides or more, 20 nucleotides or more, 25 nucleotides or more, or 30 nucleotides or more. In some embodiments, the start of the 5′ PCR handle site is 16 nucleotides 3′ of the 3′-most direct repeat of the gRNA array. In some embodiments, the start of the 5′ PCR handle site is within 16 nucleotides (i.e., is 16 nt or less) 3′ of the 3′-most direct repeat of the gRNA array. In some embodiments, the start of the 5′ PCR handle site is 15-30 nt (e.g., 15-25, 15-10, 15-18, 16-30, 16-25, 16-20, or 16-18 nt) 3′ of the 3′-most direct repeat of the gRNA array.

The number of different barcodes in a population of nucleic acids in the plurality will be dependent on the number of bases in the barcode as well as the potential number of different bases that can be present at each position. Thus, a population of nucleic acids having barcodes with two base positions, where each position can be any one of A, C, G and T, will have potentially 16 different DBRs (AA, AC, AG, etc.). A barcode may thus include 1, 2, 3, 4, 5, 6, 7, 8, 9, 10 or more bases, including 15 or more, 20 or more, 25 or more, etc. In some embodiments, the barcode comprises 8 to 15 nucleotides. In certain embodiments, the barcode is 24 bases in length.

The barcodes may be designed to be randomly generated. Alternatively, barcodes can also be selected to avoid sequences that hybridize within one another or other molecules within a reaction, to avoid sequences subject to sequencing errors, or to avoid sequences subject to confusion with sequences of other barcodes. Additionally, barcodes may be designed so that each may have a minimum Hamming distance from the other barcodes, thereby decreasing the likelihood that base-resolution mutations or read errors may interfere with the proper identification of the barcode. In some cases, barcodes are selected such that there is a Hamming distance of at least 2, 3, 4 or 5 nucleotides between each barcode in the plurality. In some embodiments, the barcodes are selected such that there is a Hamming distance of at least 4 nucleotides between each barcode in the plurality.

In some embodiments, a nucleic acid of the present disclosure includes a reverse transcription (RT) handle to facilitate capture, reverse transcription, and sequencing of RNA transcribed from the nucleic acid. A “reverse transcription handle” refers to a sequence of the nucleic acid that is a primer binding site to facilitate reverse transcription. Thus, in a suitable reverse transcription reaction, extension of the primer forms a polynucleotide that includes a sequence complementary to the template nucleic acid. In certain embodiments, the RT handle has a length of at least 4 nucleotides, 5 nucleotides, 10 nucleotides, 15 nucleotides, 20 nucleotides, 25 nucleotides, 30 nucleotides, 35 nucleotides, 40 nucleotides, 45 nucleotides, 50 nucleotides, 60 nucleotides, 70 nucleotides, 80 nucleotides, 90 nucleotides, 100 nucleotides, or a length within a range of any two of the foregoing lengths. In certain embodiments, the RT handle includes a poly(A) sequence (or tail), where poly(dT) primers would be suitable for initiating reverse transcription. In certain embodiments, the RT handle is capable of hybridizing with a polynucleotide on a substrate, e.g., an isolation bead. In one embodiment, the RT handle has a length of 7, 12, 17, or 22 nucleotides. In another embodiment, the RT handle includes a 10× Genomics Capture Sequence, or variant thereof. In one embodiment, the RT handle includes a 10× Genomics Capture Sequence CS1. In another embodiment, the RT handle includes a 10× Genomics Capture Sequence CS2.

### CRISPR-Cas Genetic Modification

As described above, the methods and compositions of the present disclosure include a nucleic acid encoding a gRNA array. In some embodiments, such nucleic acids may be used to perform CRISPR-Cas gene editing. CRISPR-Cas systems to increase expression or decrease expression of a target gene (e.g., via CRISPRa or CRISPRi, respectively) or to edit a target gene (e.g., induce a mutation in a target gene) are known in the art and any convenient system can be used. For example, when using CRISPRa or CRISPRi to modulate expression of a target gene, a guide RNA can be used that hybridizes at or near a transcription start site to inhibit (CRISPRi) or activate (CRISPRa) expression of the target gene.

As noted above, a nucleic acid that binds to a class 2 CRISPR-Cas effector protein (e.g., a Cas9 protein; a type V or type VI CRISPR-Cas protein; a Cas12 protein; etc.) (thereby forming a ribonucleoprotein complex (RNP)) and targets the complex to a specific location within a target nucleic acid is referred to herein as a “guide RNA” or “CRISPR-Cas guide nucleic acid” or “CRISPR-Cas guide RNA.” A guide RNA can be used to guide the protein to the target sequence. It is to be understood that in some cases, a hybrid DNA/RNA can be made such that a guide RNA includes DNA bases in addition to RNA bases—but the term “guide RNA” is still used herein to encompass such hybrid molecules. Suitable and exemplary guide RNAs are provided herein and design of such to target a particular nucleic acid will be readily apparent to one of skill in the art.

A wild type CRISPR-Cas effector protein (e.g., a Cas9 protein, a Cas12 protein) normally has nuclease activity that cleaves a target nucleic acid (e.g., a double stranded DNA (dsDNA)) at a target site defined by the region of complementarity between the guide sequence of the guide RNA and the target nucleic acid. In some cases, site-specific targeting to the target nucleic acid occurs at locations determined by both (i) base-pairing complementarity between the guide nucleic acid and the target nucleic acid; and (ii) a short motif referred to as the “protospacer adjacent motif” (PAM) in the target nucleic acid. For example, when a Cas9 protein binds to (in some cases cleaves) a dsDNA target nucleic acid, the PAM sequence that is recognized (bound) by the Cas9 protein is present on the non-complementary strand (the strand that does not hybridize with the targeting segment of the guide nucleic acid) of the target DNA. For Cas9, the PAM is immediately 3′ of the target sequence (i.e., is positioned downstream of the targeted sequence). In some cases, a PAM sequence has a length in a range of from 1 nt to 15 nt (e.g., 1 nt to 14 nt, 1 nt to 13 nt, 1 nt to 12 nt, 1 nt to 11 nt, 1 nt to 10 nt, 1 nt to 9 nt, 1 nt to 9 nt, 1 nt to 8 nt, 1 nt to 7 nt, 1 nt to 6 nt, 1 nt to 5 nt, 1 nt to 4 nt, 1 nt to 3 nt, 2 nt to 15 nt, 2 nt to 14 nt, 2 nt to 13 nt, 2 nt to 12 nt, 2 nt to 11 nt, 2 nt to 10 nt, 2 nt to 9 nt, 2 nt to 8 nt, 2 nt to 7 nt, 2 nt to 6 nt, 2 nt to 5 nt, 2 nt to 4 nt, 2 nt to 3 nt, 2 nt, or 3 nt).

CRISPR-Cas effector proteins (e.g., Cas9) from different species can have different PAM sequence requirements. For example, in some embodiments (e.g., when the Cas9 protein is derived from S. pyogenes or a closely related Cas9 is used; see for example, Chylinski et al., RNA Biol. 2013 May; 10(5):726-37; and Jinek et al., Science. 2012 Aug. 17; 337(6096):816-21; both of which are hereby incorporated by reference in their entirety), the PAM sequence can be NRG because the S. pyogenes Cas9 PAM (PAM sequence) is NAG or NGG (or NRG where “R” is A or G). For example, a Cas9 PAM sequence for S. pyogenes Cas9 can be: NGG, NAG, AGG, CGG, GGG, TGG, AAG, CAG, GAG, and TAG. In some cases, the PAM is NGG.

For Cas12a proteins, the PAM is immediately 5′ of the target sequence (i.e., is positioned upstream of the targeted sequence) (5′ of the non-complementary strand of the target DNA, where the complementary strand hybridizes to the guide sequence of the guide RNA while the non-complementary strand does not directly hybridize with the guide RNA). For example, the wild type protein of LbCas12a has a PAM preference of 5′-TTTV-3′, where V is A, C, or G. As such, LbCas12a can utilize the following PAMs: 5′-TTTA-3′, 5′-TTTC-3′, and 5′-TTTG-3′.

### Example PAMs for Wild Type Cas12a Proteins

PAM: 5′-TTTV-3′: LbCas12a (statistically calculated from experimental results)
PAM: 5′-TTTV-3′: AsCas12a
PAM: 5′-TTN-3′: FnCas12a, PmCas12a, MbCas12a, Mb2Cas12a,
Mb3Cas12a, TsCas12a, and BsCas12a
   where N=(A, C, G, or T); V=(A, C, or G); Y=(C or T); K=(G or T); S=(C or G)

In some cases, different CRISPR-Cas effector proteins (i.e., Cas9, Cas12, Cas13) may be advantageous to use in the various provided methods in order to capitalize on a desired feature (e.g., specific enzymatic characteristics of different CRISPR-Cas effector proteins). Different CRISPR-Cas effector proteins from different species may require different PAM sequences in the target DNA. Thus, for a particular CRISPR-Cas effector protein of choice, the PAM sequence requirement may be different than the PAM sequences described above. Various methods (including in silico and/or wet lab methods) for identification of the appropriate PAM sequence are known in the art and are routine, and any convenient method can be used.

In class 2 CRISPR systems, the functions of the effector complex (e.g., the cleavage of target DNA) are carried out by a single protein (which can be referred to as a CRISPR-Cas effector protein)—where the natural protein is an endonuclease (e.g., see Zetsche et al, Cell. 2015 Oct. 22; 163(3):759-71; Makarova et al, Nat Rev Microbiol. 2015 November; 13(11):722-36; Shmakov et al., Mol Cell. 2015 Nov. 5; 60(3):385-97; Shmakov et al., Nat Rev Microbiol. 2017 March; 15(3):169-182: “Diversity and evolution of class 2 CRISPR-Cas systems”; Koonin et al., Curr Opin Microbiol. 2017 June:37:67-78; and Makarova et al., Nat Rev Microbiol. 2020 February; 18(2):67-83). As such, the term “class 2 CRISPR-Cas protein” or “CRISPR-Cas effector protein” is used herein to encompass the effector protein from class 2 CRISPR systems—for example, type II CRISPR-Cas proteins (e.g., Cas9), type V CRISPR-Cas proteins (e.g., Cpf1/Cas12a, C2c1/Cas12b, C2C3/Cas12c, Cas12d/CasY, Cas12e/CasX), and type VI CRISPR-Cas proteins (e.g., C2c2/Cas13a, C2C7/Cas13c, C2c6/Cas13b). Class 2 CRISPR-Cas effector proteins include type II, type V, and type VI CRISPR-Cas proteins, but the term is also meant to encompass any class 2 CRISPR-Cas protein suitable for binding to a corresponding guide RNA and forming a ribonucleoprotein (RNP) complex.

Examples of CRISPR-Cas effector proteins will be readily available to one of ordinary skill in the art, and any convenient CRISPR-Cas effector protein can be used. In some embodiments, a subject CRISPR-Cas effector protein will be a Cas9 protein (e.g., Staphylococcus aureus Cas9 (saCas9), Streptococcus pyogenes Cas9 (SpyCas9), Neisseria meningitidis Cas9 (nmCas9), Streptococcus thermophilus Cas9 (stCas9), etc.). In some cases, a subject CRISPR-Cas effector protein will be a Cas12a protein (e.g., Acidaminococcus sp., strain BV3L6 (AsCas12a), LbCas12a, and FnoCas12a). Sequences for these proteins are readily available to one of ordinary skill in the art. As would be understood by one of ordinary skill in the art, many variant forms of CRISPR-Cas effector proteins are known in the art, e.g., those harboring mutations that increase specificity (e.g., decrease off-targeting), and any convenient variant can be used (e.g., HiFi Cas9 and AsCas12a ultra nuclease); see, e.g., as further described herein.

Type V CRISPR/Cas effector proteins are a subtype of Class 2 CRISPR/Cas effector proteins. For examples of type V CRISPR/Cas systems and their effector proteins (e.g., Cas12 family proteins such as Cas12a), see, e.g., Shmakov et al., Nat Rev Microbiol. 2017 March; 15(3):169-182: “Diversity and evolution of class 2 CRISPR-Cas systems.” Examples include, but are not limited to: Cas12 family (Cas12a, Cas12b, Cas12c), C2c4, C2c8, C2c5, C2c10, and C2c9; as well as CasX (Cas12e) and CasY (Cas12d). Also see, e.g., Koonin et al., Curr Opin Microbiol. 2017 June; 37:67-78: “Diversity, classification and evolution of CRISPR-Cas systems.”

As such in some cases, a subject type V CRISPR/Cas effector protein is a Cas12 protein (e.g., Cas12a, Cas12b, Cas12c). In some cases, a subject type V CRISPR/Cas effector protein is a Cas12 protein such as Cas12a, Cas12b, Cas12c, Cas12d, Cas12e, Cas12d, or Cas12e. In some cases, a subject type V CRISPR/Cas effector protein is a Cas12a protein. In some cases, a subject type V CRISPR/Cas effector protein is a Cas12b protein. In some cases, a subject type V CRISPR/Cas effector protein is a Cas12c protein. In some cases, a subject type V CRISPR/Cas effector protein is a Cas12d protein. In some cases, a subject type V CRISPR/Cas effector protein is a Cas12e protein. In some cases, a subject type V CRISPR/Cas effector protein is protein selected from: Cas12 (e.g., Cas12a, Cas12b, Cas12c, Cas12d, Cas12e), C2c4, C2c8, C2c5, C2c10, and C2c9. In some cases, a subject type V CRISPR/Cas effector protein is protein selected from: C2c4, C2c8, C2c5, C2c10, and C2c9. In some cases, a subject type V CRISPR/Cas effector protein is protein selected from: C2c4, C2c8, and C2c5. In some cases, a subject type V CRISPR/Cas effector protein is protein selected from: C2c10 and C2c9.

In some cases, the subject type V CRISPR/Cas effector protein is a naturally-occurring protein (e.g., naturally occurs in prokaryotic cells). In other cases, the Type V CRISPR/Cas effector protein is not a naturally-occurring polypeptide (e.g., the effector protein is a variant protein, a chimeric protein, includes a fusion partner, and the like). Examples of naturally occurring Type V CRISPR/Cas effector proteins include, but are not limited to, proteins encoded by any of SEQ ID NOs: 80-92, 103-112. Any Type V CRISPR/Cas effector protein can be suitable for the compositions (e.g., nucleic acids, kits, etc.) and methods of the present disclosure (e.g., as long as the Type V CRISPR/Cas effector protein forms a complex with a guide RNA and exhibits ssDNA cleavage activity of non-target ssDNAs once it is activated (by hybridization of and associated guide RNA to its target DNA).

In some cases, a type V CRISPR/Cas effector protein comprises an amino acid sequence having 20% or more sequence identity (e.g., 30% or more, 40% or more, 50% or more, 60% or more, 70% or more, 80% or more, 85% or more, 90% or more, 95% or more, 97% or more, 98% or more, 99% or more, or 100% sequence identity) with a Cas12 protein (e.g., Cas12a, Cas12b, Cas12c) (e.g., a Cas12 protein of SEQ ID NOs: 80-92, 103-112). For example, in some cases a type V CRISPR/Cas effector protein comprises an amino acid sequence having 50% or more sequence identity (e.g., 60% or more, 70% or more, 80% or more, 85% or more, 90% or more, 95% or more, 97% or more, 98% or more, 99% or more, or 100% sequence identity) with a Cas12 protein (e.g., Cas12a, Cas12b, Cas12c) (e.g., a Cas12 protein of SEQ ID NOs: 80-92, 103-112). In some cases a type V CRISPR/Cas effector protein comprises an amino acid sequence having 80% or more sequence identity (e.g., 85% or more, 90% or more, 95% or more, 97% or more, 98% or more, 99% or more, or 100% sequence identity) with a Cas12 protein (e.g., Cas12a, Cas12b, Cas12c) (e.g., a Cas12 protein of SEQ ID NOs: 80-92, 103-112). In some cases a type V CRISPR/Cas effector protein comprises an amino acid sequence having 90% or more sequence identity (e.g., 95% or more, 97% or more, 98% or more, 99% or more, or 100% sequence identity) with a Cas12 protein (e.g., Cas12a, Cas12b, Cas12c) (e.g., a Cas12 protein of SEQ ID NOs: 80-92, 103-112). In some cases a type V CRISPR/Cas effector protein comprises a Cas12 amino acid sequence (e.g., Cas12a, Cas12b, Cas12c) of SEQ ID NOs: 80-92, 103-112.

In some cases, CRISPR-Cas effector proteins of the present disclosure are variant CRISPR-Cas effector proteins. A variant CRISPR-Cas effector protein of the present disclosure (i.e., a subject variant CRISPR-Cas effector protein) comprises an amino acid sequence having one or more amino acid substitutions relative to a corresponding wild-type CRISPR-Cas effector protein. In some cases, the one or more amino acid substitutions will modify the activity of the variant CRISPR-Cas effector protein relative to the wild type CRISPR-Cas effector protein.

As would be understood by one of ordinary skill in the art, many variant forms of CRISPR-Cas effector proteins are known in the art, e.g., those harboring mutations that increase specificity (e.g., decrease off-targeting), and any convenient variant can be used (e.g., AsCas12a ultra nuclease) in combination with the variants of the present disclosure. See, e.g., Vakulskas et al., Nat Med. 2018 August; 24(8): 1216-1224; Kleinstiver et al., Nature. 2016 Jan. 28; 529(7587):490-5; Yuen et al., Nucleic Acids Res. 2022 Feb. 22; 50(3):1650-1660; Wei et al., FASEB J. 2023 August; 37(8):e23060; Tan et al., Proc Natl Acad Sci USA. 2019 Oct. 15; 116(42):20969-20976; Kleinstiver et al., Nat Biotechnol. 2019 March; 37(3):276-282; DeWeirdt et al., Nat. Biotechnol. 2021 39, 94-104; and Zhang et al., Nat Commun. 2021 Jun. 23; 12(1):3908.

In the present disclosure, a subject CRISPR-Cas effector protein (e.g., a Cas9 protein, a Cas12 protein such as a Cas12a protein, a Cas13 protein, and the like) has reduced catalytic activity (e.g., a Cas9 protein with nickase activity (nCas9), a dead Cas9 protein (dCas9), a dead Cas12 protein (dCas12)). When the protein has nickase activity, it can be referred to as a nickase or an “nCas” (e.g., nCas9). When the protein binds to but does not cleave the target nucleic acid, it is referred to as “catalytic inactive”, which is used interchangeably with the term “dead”. A dead CRISPR-Cas effector protein can also be referred to as a “dCas” (e.g., dCas9, dCas12 such as dCas12a, dCas13, and the like).

For example, when a Cas9 protein has a mutation at one or more amino acid positions corresponding to D10, G12, G17, E762, H840, N854, N863, H982, H983, A984, D986, and/or a A987 of the Cas9 protein set forth in SEQ ID NO: 26 (e.g., D10A, G12A, G17A, E762A, H840A, N854A, N863A, H982A, H983A, A984A, and/or D986A), the variant Cas9 protein can still bind to target DNA in a site-specific manner (because it is still guided to a target DNA sequence by a guide RNA) as long as it retains the ability to interact with the guide RNA. In some cases, the CRISPR-Cas effector protein of a subject CRISPR-Cas fusion protein is a nickase (e.g., cleaves one strand of a double stranded target nucleic acid but not the other strand) (e.g., the Cas9 protein can be a nickase, e.g., can include one or more amino acid mutations that make it a nickase). For example, in some cases, a Cas9 protein of a subject CRISPR-Cas fusion protein has a mutation in a catalytic domain (e.g., a mutation in a RuvC or HNH domain).

For example, in some cases, a CRISPR-Cas effector protein (e.g., a Cas9 protein) can cleave the complementary strand of a target nucleic acid but has reduced ability to cleave the non-complementary strand of a target nucleic acid. For example, the Cas9 protein can have a mutation (amino acid substitution) that reduces the function of the RuvC domain. Thus, the Cas9 protein can be a nickase that cleaves the complementary strand, but does not cleave the non-complementary strand. As a non-limiting example, in some cases, a Cas9 protein has a mutation at residue D10 (e.g., D10A, aspartate to alanine) of SEQ ID NO: 26 (or the corresponding position of any Cas9 protein, e.g., any of the proteins set forth in SEQ ID NOs: 26-61) and can therefore cleave the complementary strand of a double stranded target nucleic acid but has reduced ability to cleave the non-complementary strand of a double stranded target nucleic acid (thus resulting in a single strand break (SSB) instead of a double strand break (DSB) when the variant Cas9 protein cleaves a double stranded target nucleic acid) (see, for example, Jinek et al., Science. 2012 Aug. 17; 337(6096):816-21). Examples of such amino acid positions in a RuvC domain can include: D10, G12, G17, E762, H982, H983, A984, D986, and/or A987 of the Cas9 protein set forth in SEQ ID NO: 26 (e.g., D10A, G12A, G17A, E762A, H982A, H983A, A984A, and/or D986A).

In some cases, a CRISPR-Cas effector protein (e.g., a Cas9 protein) can cleave the non-complementary strand of a target nucleic acid but has reduced ability to cleave the complementary strand of the target nucleic acid. For example, the Cas9 protein can have a mutation (amino acid substitution) that reduces the function of the HNH domain. Thus, the Cas9 protein can be a nickase that cleaves the non-complementary strand, but does not cleave the complementary strand (e.g., does not cleave a single stranded target nucleic acid). As a non-limiting example, in some embodiments, the Cas9 protein has a mutation at position H840 (e.g., an H840A mutation, histidine to alanine) of SEQ ID NO: 26 (or the corresponding position of any Cas9 protein, e.g., the Cas9 proteins set forth as SEQ ID NOs: 26-61 and can therefore cleave the non-complementary strand of the target nucleic acid but has reduced ability to cleave (e.g., does not cleave) the complementary strand of the target nucleic acid. Such a Cas9 protein has a reduced ability to cleave a target nucleic acid (e.g., a single stranded target nucleic acid). Examples of such amino acid positions in an HNH domain can include: H840, N854, and/or N863 of the Cas9 protein set forth in SEQ ID NO: 26 (e.g., H840A, N854A, and/or N863A).

In some cases, a CRISPR-Cas effector protein (e.g., a Cas9 protein) has a reduced ability to cleave both the complementary and the non-complementary strands of a double stranded target nucleic acid. In some cases, the CRIPSR-Cas effector protein is a dead Cas protein (also referred to as catalytically inactive) and thus can be referred to as a dCas (e.g., dCas9, dCas12, and the like). As a non-limiting example, in some cases, a dCas9 protein harbors mutations at residues D10 and H840 (e.g., D10A and H840A) of SEQ ID NO: 26 (or the corresponding residues of another Cas9 protein, e.g., any of the proteins set forth as SEQ ID NOs: 26-61) such that the polypeptide has a reduced ability to cleave (e.g., does not cleave) both the complementary and the non-complementary strands of a target nucleic acid. Such a Cas9 protein has a reduced ability to cleave a target nucleic acid (e.g., a single stranded or double stranded target nucleic acid) but retains the ability to bind a target nucleic acid. For example, a Cas9 protein of a subject Cas9 fusion polypeptide can have a mutation in one or more of amino acid positions in (i) a RuvC domain: corresponding to D10, G12, G17, E762, H982, H983, A984, D986, and/or A987 of the Cas9 protein set forth in SEQ ID NO: 26 (e.g., D10A, G12A, G17A, E762A, H982A, H983A, A984A, and/or D986A); and one or more of amino acid positions in (ii) an HNH domain: corresponding to H840, N854, and/or N863 of the Cas9 protein set forth in SEQ ID NO: 26 (e.g., H840A, N854A, and/or N863A). In some cases, the CRIPSR-Cas effector protein is a dCas12a protein (see, e.g., Cheng et al., Plant Commun. 2023 Jul. 10; 4(4):100601, which used a fusion protein of dCas12a fused to TadA8e). With respect to Cas12, examples of a single mutation in the nuclease domain, such as D832A or E925A or both (D832A/E925A), can cause it to become catalytically inactive (e.g., dCas12a). Another example is E993A of Cas12a. See, e.g., Kang et al., bioRxiv [Preprint]. 2023 May 8:2023.05.08.539911; Ciurkot et al., Nucleic Acids Research, Volume 49, Issue 13, 21 Jul. 2021, Pages 7775-7790; Campa et al., Nat Methods 16, 887-893 (2019).

In some cases, a CRISPR-Cas effector protein (e.g., a Cas9 protein) is a variant. In some cases, such a variant is a high fidelity (HF) protein such as a HF Cas9 protein (also referred to as SpCas9-HF1 or HF1- and also -HF2, -HF3, -HF4) (e.g., see Kleinstiver et al. (2016) Nature 529:490). For example, amino acids N497, R661, Q695, and Q926 of the amino acid sequence set forth as (SEQ ID NO: 26) (or the corresponding position of another Cas9 protein, e.g., a protein having the amino acid sequence of any of the sequences set forth as SEQ ID NOs: 26-61) can be substituted, e.g., with alanine. In some cases, a suitable parent Cas9 protein exhibits altered PAM specificity, e.g., in some cases the Cas9 is a VRVRFRR Cas9 (which recognizes an NG PAM instead of NGG; see, e.g., SEQ ID NO: 63) see, e.g., Nishimasu et al., Science. 2018 Sep. 21; 361(6408):1259-1262; and Kleinstiver et al. (2015) Nature 523:481. Additional examples of Cas9 variants that can be used, include, but are not limited to: HiFiCas9 (e.g., R691A), eSpCas9 (e.g., K810A, K1003A, R1060A), eSpCas9 (e.g., D1135E), HypaCas9 (e.g., N692A, M694A, Q695A, H698A), xCas9 (e.g., E108G, S217A, A262T, S4091, E480K, E543D, M6941, E1219V), Sniper-Cas9 (e.g., F539S, M763I, K890N), evoCas9 (e.g., M495V, Y515N, K526E, R661Q), SpartaCas (e.g., D23A, T67L, Y128V, D1251G), LZ3Cas9 (e.g., N690C, T7691, G915M, N980K), miCas9 (e.g., SV40 NLS linker fused with brex27 motif), SuperFi-Cas9 (e.g., Y1010D, Y1013D, Y1016D, V1018D, R1019D, Q1027D, K1031D) (see, e.g., Allemailem et al, Int J Mol Sci. 2023 Apr. 11; 24(8):7052) [relative to SEQ ID NO: 26].

A variant CRISPR-Cas effector protein (e.g., Cas9, Cas12, Cas13) can be fused to a heterologous protein having any desired activity such as nucleic acid-modifying activity (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity (e.g., reverse transcriptase activity), ligase activity, helicase activity, photolyase activity or glycosylase activity); transcription modulation activity (e.g., fusion to a transcription repressor or transcription activator); an activity that modifies a protein (e.g., a histone) that is associated with target DNA or RNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity). A heterologous polypeptide can also be referred to herein as a “fusion partner.”

In some such cases the CRISPR-Cas effector protein harbors a mutation that reduces the endogenous nuclease activity (e.g., in some cases renders it a nickase and in some cases renders it catalytically inactive (“dead), e.g., a dCas9). In some cases, a CRISPR-Cas effector protein (e.g., a nickase or ‘dead’ version) is fused to a heterologous protein that has transcription activation activity (e.g., includes a transcription activator domain) and thereby increases transcription (and therefore expression) of a target gene (referred to as CRISPRa). In some cases, a CRISPR-Cas effector protein (e.g., a nickase or ‘dead’ version) is fused to a heterologous protein that has transcription repressor activity (e.g., includes a transcription repression domain) and thereby reduces transcription (and therefore expression) of a target gene (referred to as CRISPRi).

Examples of proteins (or fragments thereof) that can be used to increase transcription (often referred to as CRISPRa when used with a CRISPR-Cas effector protein) include but are not limited to: transcriptional activators such as VP16, VP64, VP48, VP64, VP160, p65 subdomain (e.g., from NFkB), Rta, VPR (which is a fusion of VP64, p65, and Rta), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROS1, and the like. See, e.g., Chavez et al., Nat Methods. 2015 April; 12(4):326-328. In some cases, the CRISPRa system is a SAM system, which includes 3 components that form the DNA-binding complex: (1) a CRISPRa fusion protein (e.g., dCas9 fused to VP64), (2) MS2 aptamer(s) added to the guide RNA (forming a characteristic stem loop structure recognized by MS2), and (3) transcriptional activators P65 (Nuclear Factor NF-κB p65) and HSF1 (Heat Shock Factor 1) fused with an MS2-tag corresponding to the minimal aptamer-binding peptide of the MS2 coat protein. See, e.g., review articles such as Adli, Nat Commun. 2018 May 15; 9(1):1911; Becirovic, Cell Mol Life Sci. 2022 Feb. 12; 79(2):130; and Nidhi S, et al., Int J Mol Sci. 2021 Mar. 24; 22(7):3327.

Examples of proteins (or fragments thereof) that can be used as heterologous proteins to decrease transcription (often referred to as CRISPRi when used with a CRISPR-Cas effector protein) include but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In some cases, a programmable genome editing protein such as a CRISPR-Cas effector protein (e.g., Cas9, Cas12a, Cas13) is fused to one or more heterologous polypeptides (fusion partners) that provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an endoplasmic reticulum (ER) retention signal, and the like). In some cases, the CRISPR-Cas effector protein does not include an NLS.

In some cases, the heterologous protein fused to the CRISPR-Cas effector protein is one or more heterologous nuclear localization signals (NLSs). In some cases, 1 to 10 NLSs (e.g., 1-9, 1-8, 1-7, 1-6, 1-5, 2-10, 2-9, 2-8, 2-7, 2-6, 2-5, 4-10, 4-9, 4-8, 4-7, 5-10, 5-9, or 5-8 NLSs). In some cases, 2 to 5 NLSs (e.g., 2-4 NLSs, or 2-3 NLSs). In some cases, about 4 NLSs. In some cases, about 7 NLSs. Non-limiting examples of NLSs include an NLS sequence derived from: the NLS of the SV40 virus large T-antigen, having the amino acid sequence PKKKRKV (SEQ ID NO: 64); the NLS from nucleoplasmin (e.g., the nucleoplasmin bipartite NLS with the sequence KRPAATKKAGQAKKKK (SEQ ID NO: 65)); the c-myc NLS having the amino acid sequence PAAKRVKLD (SEQ ID NO: 66) or RQRRNELKRSP (SEQ ID NO: 67); the hRNPA1 M9 NLS having the sequence NQSSNFGPMKGGNFGGRSSGPYGGGGQYFAKPRNQGGY (SEQ ID NO: 68); the sequence RMRIZFKNKGKDTAELRRRRVEVSVELRKAKKDEQILKRRNV (SEQ ID NO: 69) of the IBB domain from importin-alpha; the sequences VSRKRPRP (SEQ ID NO: 70) and PPKKARED (SEQ ID NO: 71) of the myoma T protein; the sequence PQPKKKPL (SEQ ID NO:72) of human p53; the sequence SALIKKKKKMAP (SEQ ID NO: 73) of mouse c-abl IV; the sequences DRLRR (SEQ ID NO: 74) and PKQKKRK (SEQ ID NO: 75) of the influenza virus NS1; the sequence RKLKKKIKKL (SEQ ID NO: 76) of the Hepatitis virus delta antigen; the sequence REKKKFLKRR (SEQ ID NO: 77) of the mouse Mx1 protein; the sequence KRKGDEVDGVDEVAKKKSKK (SEQ ID NO: 78) of the human poly(ADP-ribose) polymerase; and the sequence RKCLQAGMNLEARKTKK (SEQ ID NO: 79) of the steroid hormone receptors (human) glucocorticoid.

In general, NLS (or multiple NLSs) are of sufficient strength to drive accumulation of a subject protein in the nucleus of a eukaryotic cell. If desired, detection of accumulation in the nucleus may be performed by any suitable technique. For example, a detectable marker may be fused to the polypeptide such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly. In some cases, the heterologous polypeptide can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some embodiments, a CRISPR-Cas effector protein (e.g., Cas9, Cas12a, Cas13) can be fused to a fusion partner via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G) n where n is an integer of at least one; glycine-serine polymers (including, for example, (GS) n, (GSGGS) n (SEQ ID NO: 133), (GGSGGS) n (SEQ ID NO: 134), (GGGGS) n (SEQ ID NO: 135), and (GGGS) n (SEQ ID NO: 136), where n is an integer of at least one; e.g., where n is an integer from 1 to 10); glycine-alanine polymers; and alanine-serine polymers. Exemplary linkers can comprise amino acid sequences including, but not limited to, GGSG (SEQ ID NO: 126), GGSGG (SEQ ID NO: 127), GSGSG (SEQ ID NO: 128), GSGGG (SEQ ID NO: 129), GGGSG (SEQ ID NO: 130), GSSSG (SEQ ID NO: 131), GGGGS (SEQ ID NO: 132), and the like. The ordinarily skilled artisan will recognize that design of a peptide conjugated to any desired element can include linkers that are all or partially flexible, such that the linker can include a flexible linker as well as one or more portions that confer less flexible structure.

In some cases, a nucleotide sequence encoding a CRISPR-Cas effector protein is codon optimized. This type of optimization can entail a mutation of a CRISPR-Cas effector protein-encoding nucleotide sequence to mimic the codon preferences of the intended host organism or cell while encoding the same protein. Thus, the codons can be changed, but the encoded protein remains unchanged. For example, if the intended target cell was a human cell, a human codon-optimized CRISPR-Cas effector protein-encoding nucleotide sequence could be used. As another non-limiting example, if the intended host cell were a mouse cell, then a mouse codon-optimized CRISPR-Cas effector protein-encoding nucleotide sequence could be generated. As another non-limiting example, if the intended host cell were a plant cell, then a plant codon-optimized CRISPR-Cas effector protein-encoding nucleotide sequence could be generated. As another non-limiting example, if the intended host cell were an insect cell, then an insect codon-optimized CRISPR-Cas effector protein-encoding nucleotide sequence could be generated.

Codon usage tables are readily available, for example, at the “Codon Usage Database” available at www[dot]kazusa[dot] or [dot]jp[forwardslash]codon. In some cases, a nucleic acid of the present disclosure comprises a CRISPR-Cas effector protein-encoding nucleotide sequence that is codon optimized for expression in a eukaryotic cell. In some cases, a nucleic acid of the present disclosure comprises a CRISPR-Cas effector protein-encoding nucleotide sequence that is codon optimized for expression in an animal cell. In some cases, a nucleic acid of the present disclosure comprises a CRISPR-Cas effector protein-encoding nucleotide sequence that is codon optimized for expression in a fungus cell. In some cases, a nucleic acid of the present disclosure comprises a CRISPR-Cas effector protein-encoding nucleotide sequence that is codon optimized for expression in a plant cell.

For additional information related to programmable gene editing tools (e.g., CRISPR-Cas RNA-guided proteins such as Cas9, CasX, CasY, Cas12a, Cas13, Zinc finger proteins such as Zinc finger nucleases, TALE proteins such as TALENs, CRISPR-Cas guide RNAs, PAMs, and the like) refer to, for example, Dreier, et al., (2001) J Biol Chem 276:29466-78; Dreier, et al., (2000) J Mol Biol 303:489-502; Liu, et al., (2002) J Biol Chem 277:3850-6); Dreier, et al., (2005) J Biol Chem 280:35588-97; Jamieson, et al., (2003) Nature Rev Drug Discov 2:361-8; Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29:183-212; Segal and Barbas, (2001) Curr Opin Biotechnol 12:632-7; Segal, et al., (2003) Biochemistry 42:2137-48; Beerli and Barbas, (2002) Nat Biotechnol 20:135-41; Carroll, et al., (2006) Nature Protocols 1:1329; Ordiz, et al., (2002) Proc Natl Acad Sci USA 99:13290-5; Guan, et al., (2002) Proc Natl Acad Sci USA 99:13296-301; Sanjana et al., Nature Protocols, 7:171-192 (2012); Zetsche et al, Cell. 2015 Oct. 22; 163(3):759-71; Makarova et al, Nat Rev Microbiol. 2015 November; 13(11):722-36; Shmakov et al., Mol Cell. 2015 Nov. 5; 60(3):385-97; Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al, Cell. 2013 Feb. 28; 152(5):1173-83; Wang et al., Cell. 2013 May 9; 153(4):910-8; Auer et. al., Genome Res. 2013 Oct. 31; Chen et. al., Nucleic Acids Res. 2013 Nov. 1; 41(20):e19; Cheng et. al., Cell Res. 2013 October; 23(10):1163-71; Cho et. al., Genetics. 2013 November; 195(3):1177-80; DiCarlo et al., Nucleic Acids Res. 2013 April; 41(7):4336-43; Dickinson et. al., Nat Methods. 2013 October; 10(10):1028-34; Ebina et. al., Sci Rep. 2013; 3:2510; Fujii et. al, Nucleic Acids Res. 2013 Nov. 1; 41(20):e187; Hu et. al., Cell Res. 2013 November; 23(11):1322-5; Jiang et. al., Nucleic Acids Res. 2013 Nov. 1; 41(20):e188; Larson et. al., Nat Protoc. 2013 November; 8(11):2180-96; Mali et. at., Nat Methods. 2013 October; 10(10):957-63; Nakayama et. al., Genesis. 2013 December; 51(12):835-43; Ran et. al., Nat Protoc. 2013 November; 8(11):2281-308; Ran et. al., Cell. 2013 Sep. 12; 154(6):1380-9; Upadhyay et. al., G3 (Bethesda). 2013 Dec. 9; 3(12):2233-8; Walsh et. al., Proc Natl Acad Sci USA. 2013 Sep. 24; 110(39):15514-5; Xie et. al., Mol Plant. 2013 Oct. 9; Yang et. al., Cell. 2013 Sep. 12; 154(6):1370-9; Briner et al., Mol Cell. 2014 October 23; 56(2):333-9; Burstein et al., Nature. 2016 Dec. 22-Epub ahead of print; Gao et al., Nat Biotechnol. 2016 July 34(7):768-73; Shmakov et al., Nat Rev Microbiol. 2017 March; 15(3):169-182; Makarova et al., Nat Rev Microbiol. 2020 February; 18(2):67-83; as well as international patent application publication Nos. WO2002099084; WO00/42219; WO02/42459; WO2003062455; WO03/080809; WO05/014791; WO05/084190; WO08/021207; WO09/042186; WO09/054985; and WO10/065123; U.S. patent application publication Nos. 20030059767, 20030108880, 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; 20140377868; 20150166983; and 20160208243; and U.S. Pat. Nos. 6,140,466; 6,511,808; 6,453,242 8,685,737; 8,906,616; 8,895,308; 8,889,418; 8,889,356; 8,871,445; 8,865,406; 8,795,965; 8,771,945; and 8,697,359; all of which are hereby incorporated by reference in their entirety.

In some cases, a gene editing composition includes a CRISPR-Cas base editor (e.g. Komor et al (2016) Nature. 533(7603):420-424. doi: 10.1038/nature17946). In some cases, a gene editing composition includes a CRISPR-Cas prime editor (e.g., Anzalone et al (2019) Nature 576:149-157 https://doi.org/10.1038/s41586-019-1711-4).

In some embodiments, the present disclosure provides a CRISPR-Cas effector polypeptide as a protein. In some cases, the present disclosure provides a cell engineered to express the CRISPR-Cas effector protein (e.g., a mammalian cell line that stably expresses Cas12). In some instances, the present disclosure provides a nucleic acid comprising a nucleotide sequence encoding a CRISPR-Cas effector polypeptide. In some cases, the present disclosure provides a nucleic acid encoding one or more polypeptides and a gRNA array. In some instances, the one or more polypeptides includes a CRISPR-Cas effector polypeptide. In some cases, the one or more polypeptides includes a cell marker (e.g., a fluorescent protein such as BFP, a selectable marker such as a puromycin resistance gene). In some embodiments, the nucleic acid includes a sequence encoding an RNA stabilizing element. In some cases, the RNA stabilizing element is a MALAT1 triplex structure. In some embodiments, the nucleic acid of the present disclosure does not include SEQ ID NO: 3 between the end of the MALAT1 triplex sequence and the start of the gRNA array sequence. In some cases, the start of the gRNA array sequence is positioned within 40 nucleotides of the end of the MALAT1 triplex sequence. In some cases, the nucleotide sequence(s) encoding the component(s) (e.g., a CRISPR-Cas effector polypeptide, a cell marker, a MALAT1 triplex structure, a gRNA array) of the nucleic acid of the present disclosure is operably linked to a single promoter. In some embodiments, the promoter is a Pol II promoter.

In some cases, the CRISPR-Cas effector polypeptide is a Type II CRISPR-Cas effector polypeptide. In some cases, the CRISPR-Cas effector polypeptide is a Cas9 polypeptide. In some cases, the Cas9 polypeptide is a Streptococcus pyogenes Cas9 (spyCas9) polypeptide. In some cases, the CRISPR-Cas effector protein is a Cas12a. Examples of Cas12a proteins include, but are not limited to, those of SEQ ID NOs: 80-92. In some cases, the CRISPR-Cas effector protein is a Cas13. Examples of Cas13 proteins include, but are not limited to, those of SEQ ID NOs: 93-102.

Introducing Components into a Target Cell

A gRNA array of the present disclosure (e.g., a gRNA array encoded by a nucleic acid of the present disclosure) and/or a CRISPR-Cas effector protein (or a nucleic acid encoding a CRISPR-Cas effector polypeptide) can be introduced into a host cell by any of a variety of well-known methods.

A gRNA array can be introduced, e.g., as a DNA molecule encoding the guide RNA array, or can be provided directly as an RNA molecule (or a hybrid molecule when applicable). In some cases, a CRISPR-Cas effector protein is provided as a nucleic acid (e.g., an mRNA, a DNA, a plasmid, an expression vector, a viral vector, etc.) that encodes the protein. In some cases, the CRISPR-Cas effector protein is provided directly as a protein (e.g., without an associated guide RNA or with an associate guide RNA, i.e., as a ribonucleoprotein complex-RNP). Like a gRNA array, a CRISPR-Cas effector protein can be introduced into a cell (provided to the cell) by any convenient method; such methods are known to those of ordinary skill in the art. As an illustrative example, a CRISPR-Cas effector protein can be injected directly into a cell (e.g., with or without a guide RNA array or nucleic acid encoding a guide RNA array). As another example, a preformed complex of a CRISPR-Cas effector protein and a guide RNA (an RNP) can be introduced into a cell (e.g., eukaryotic cell) (e.g., via injection, via nucleofection; via a protein transduction domain (PTD) conjugated to one or more components, e.g., conjugated to the CRISPR-Cas effector protein, conjugated to a guide RNA; etc.).

Methods of introducing a nucleic acid and/or protein into a cell are known in the art, and any convenient method can be used to introduce a subject nucleic acid (e.g., an expression construct/vector) into a target cell (e.g., prokaryotic cell, eukaryotic cell, plant cell, animal cell, mammalian cell, human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: 50169-409X(12) 00283-9. doi: 10.1016/j.addr.2012.09.023), and the like.

Nucleic acids may be provided to the cells using well-developed transfection techniques; see, e.g. Angel and Yanik (2010) PLOS ONE 5(7):e11756, and the commercially available TransMessenger® reagents from Qiagen, Stemfect™ RNA Transfection Kit from Stemgent, and TransIT®-mRNA Transfection Kit from Mirus Bio LLC. See also Beumer et al. (2008) PNAS 105(50):19821-19826.

Vectors may be provided directly to a target host cell. In other words, in some cases, a target host cell is contacted with one or more recombinant expression vectors comprising the subject nucleic acids (e.g., recombinant expression vectors having the encoding the guide RNA array; recombinant expression vectors encoding the CRISPR-Cas effector polypeptide or cell marker polypeptides; etc.) such that the vectors are taken up by the cells. Methods for contacting cells with nucleic acid vectors that are plasmids, include electroporation, calcium chloride transfection, microinjection, and lipofection are well known in the art. For viral vector delivery, cells can be contacted with viral particles comprising the subject viral expression vectors.

Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

In some cases, a nucleic acid (e.g., a guide RNA array; a nucleic acid comprising a nucleotide sequence encoding a CRISPR-Cas effector protein; etc.) and/or a polypeptide (e.g., a CRISPR-Cas effector protein) is delivered to a cell (e.g., a target host cell) in a particle, or associated with a particle. The terms “particle” and “nanoparticle” can be used interchangeably, as appropriate. As a non-limiting example, a gRNA array and/or a CRISPR-Cas effector protein can be combined with a lipid. As another non-limiting example, a gRNA array and/or a CRISPR-Cas effector protein can be combined with a particle, or formulated into a particle.

Introducing a nucleic acid (e.g., a guide RNA array; a nucleic acid comprising a nucleotide sequence encoding a CRISPR-Cas effector protein; etc.) and/or a polypeptide (e.g., a CRISPR-Cas effector protein) into cells can occur in any culture media and under any culture conditions that promote the survival of the cells. Introducing the nucleic acid and/or the polypeptide into a target cell can be carried out in vivo or ex vivo. Introducing the nucleic acid and/or the polypeptide into a target cell can be carried out in vitro.

### Cells

Suitable target cells (which can comprise target nucleic acids such as genomic DNA) (i.e., host cells) include, but are not limited to: a bacterial cell; an archaeal cell; a cell of a single-cell eukaryotic organism; a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell from a vertebrate animal (e.g., a fish, an amphibian, a reptile, a bird, a mammal); a cell from a mammal (e.g., a cell from a rodent; a cell from a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuna, a sheep, a goat, etc.); a cell of a marine mammal (e.g., a whale, a seal, an elephant seal, a dolphin, a sea lion; etc.) and the like. Any type of cell may be of interest (e.g. a stem cell, e.g. an embryonic stem (ES) cell, an induced pluripotent stem (iPS) cell, a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.).

Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in vitro. Target cells can be unicellular organisms and/or can be grown in culture. If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. can be conveniently harvested by biopsy. In some cases, the cell line comprises cells that are engineered to stably express a CRISPR-Cas effector protein (e.g., Cas9, Cas12, Cas13).

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

A cell can be an in vitro cell (e.g., established cultured cell line). A cell can be an ex vivo cell (cultured cell from an individual). A cell can be and in vivo cell (e.g., a cell in an individual). A cell can be an isolated cell. A cell can be a cell inside of an organism. A cell can be an organism (e.g., a single cell eukaryotic organism).

### Methods

The present disclosure provides methods of using a nucleic acid, e.g., a DNA molecule encoding a gRNA array, a 5′ PCR handle, and a barcode, e.g., as described above.

In some embodiments, a nucleic acid of the present disclosure can be used to express, under the control of a single promoter, a polypeptide and one or more gRNAs from a CRISPR-Cas gRNA array (e.g., in some cases, for the purpose of performing a CRISPR based screen). In some cases, the components of the nucleic acid (e.g., one or more polypeptides, a MALAT1 triplex structure, a gRNA array, a barcode) are operably linked to a single promoter. In certain embodiments, the promoter is a Pol II promoter (e.g., an EF1a promoter). As such, methods of the present disclosure include initiating expression (e.g., of one or more polypeptides, of a MALAT1 triplex structure, of a gRNA array, of a barcode) from the Pol II promoter (e.g., EF1a) of a nucleic acid of the present disclosure. In some cases, the polypeptides expressed from the Pol II promoter include, e.g., a fluorescent protein, an antibiotic resistance protein, and/or a CRISPR-Cas effector protein. In some embodiments, the methods include expressing two or more polypeptides (e.g., a fluorescent protein and an antibiotic resistance protein) from the Pol II promoter. In some embodiments, initiating expression from a Pol II promoter of a nucleic acid of the present disclosure stabilizes expression of the one or more polypeptides and/or stabilizes or increases production of the gRNAs of the array.

In some cases, methods of the present disclosure include introducing a nucleic acid of the present disclosure and/or other components (e.g., a nucleic acid encoding a CRISPR-Cas effector polypeptide, a CRISPR-Cas effector protein) into a cell. Methods of introducing a nucleic acid and/or other components into a cell, as well as suitable cell types, are known in the art and described in greater detail above. In some instances, the cell expresses a CRISPR-Cas effector protein. In some cases, the methods include providing a CRISPR-Cas effector protein or a nucleotide sequence encoding a CRISPR-Cas effector protein to the cell.

Methods of the present disclosure also include the use of a nucleic acid, e.g., as described above, to perform CRISPR-Cas gene editing or modification, such as, e.g., in CRISPR-Cas systems to increase expression or decrease expression of a target gene (e.g., via CRISPRa or CRISPRi, respectively) or to edit a target gene (e.g., induce a mutation in a target gene). As described above, a nucleic acid of the present disclosure can be used to express one or more gRNAs from a CRISPR-Cas gRNA array. In combination with the appropriate CRISPR-Cas effector protein (e.g., a Cas9 protein, a Cas12 protein, a Cas13 protein, or a variant thereof), which is readily identified and selected by those skilled in the art, each gRNA expressed from a nucleic acid of the present disclosure binds to a CRISPR-Cas effector protein, forming a ribonucleoprotein (RNP) complex, and targets the complex to a specific target sequence within a target nucleic acid. The CRISPR-Cas effector protein may be provided as a nucleotide sequence encoding the polypeptide as part of the nucleic acid encoding the gRNAs, as a nucleotide sequence as part of a separate nucleic acid, or as a protein, or the cell may express a CRISPR-Cas effector protein. Depending on the selection of the CRISPR-Cas effector protein, various types of gene editing and genetic modification can be achieved, e.g., as described above, which can be useful in a variety of methods including, but not limited to, treating a disease associated with an abnormal RNA; screening functional RNA(s); knocking-down, detecting, or editing a target RNA; or detecting or editing splicing, alternative isoforms, intron retention or differential UTR usage, or binding but not degrading the target.

Also provided are compositions and methods for multiplex CRISPR-based screening. Such methods can be used to identify target sequences (e.g., genes) of interest that play roles in phenotypes such as cell survival, cell death, cell proliferation, etc. and systematically interrogate gene function and regulatory networks. For more information related to multiplex CRISPR-based screening, see, e.g., Wessels, H H., et al. Efficient combinatorial targeting of RNA transcripts in single cells with Cas13 RNA Perturb-seq. Nat Methods 20, 86-94 (2023); Snetkova V. et al. Degron-modified Cas12a enhances single-cell CRISPR screening. bioRxiv 2024.12.08.627374 (2024). doi: https://doi.org/10.1101/2024.12.08.627374; as well as US patent application publication No. US20240124873A1; which are incorporated herein by reference.

The method includes providing to a cell a subject nucleic acid comprising a CRISPR-Cas gRNA array as described herein and a CRISPR-Cas effector protein, wherein the CRISPR-Cas effector protein, guided by the gRNAs of the array, introduces one or more perturbations in the cell transcriptome. In some cases, the CRISPR-Cas effector protein is the polypeptide that is operably linked to the same promoter as the gRNA array. In some case, the CRISPR-Cas effector protein is separate from the gRNA (i.e., is not operably linked to the same promoter) (e.g., can be provided on a different nucleic acid), and in some such cases, a different protein such as cell marker protein is operably linked to the same Pol-II promoter as the gRNA array.

In some embodiments, a plurality of nucleic acids of the present disclosure, each encoding a gRNA array, is contacted with a plurality of cells (introduced to a plurality of cells). In some cases, each nucleic acid includes a different gRNA array, i.e., the gRNA array of each nucleic acid includes one or more different gRNAs than the gRNA array of another nucleic acid of the plurality. In each cell, in combination with a CRISPR-Cas effector protein (either provided to, or expressed by, the cell, e.g., as described above), the gRNAs of each nucleic acid form RNP complexes and target the complexes to one or more target sequences in one or more target nucleic acids, whereupon the RNP complexes introduces a gene edit or modification (e.g., as determined by the selection of a CRISPR-Cas effector protein, as noted above). In other words, each gRNA array encodes multiple perturbations that, in conjunction with a CRISPR-Cas effector protein, allows for the simultaneous targeting of multiple target sequences and/or multiple genes, and each cell of the plurality may be modified with a different set of perturbations than another cell of the plurality.

In some embodiments, the method reduces level of one or more of target RNA(s) in a target cell. In a further embodiment, the method functionally knocks down or knocks out one or more gene(s) expressing the target RNA(s). In yet a further embodiment, the method knocks down or knocks out one or more gene(s) in a plurality of targets cells in parallel.

In some embodiments, the gRNA arrays of the nucleic acids of the present disclosure may include gRNAs selected from a nucleic acid library. The nucleic acid library can comprise a targeted collection of gRNAs for targeting a desired set or type of genes or genomic loci. For example, the nucleic acid library can comprise gRNAs designed for exon-targeting, intron targeting, 5′ and/or 3′ UTR targeting, gene pair targeting library, dual-targeting of individual genes library, enhancer targeting library, promoter targeting library and/or non-coding RNA targeting. Accordingly, in one embodiment, the nucleic acid library is selected from an exon-targeting library, an intron-targeting library, a 5′ and/or 3′ UTR targeting library, a paralog targeting library, a chromosome targeting library, gene pair targeting library, dual-targeting of individual genes library, enhancer targeting library, promoter targeting library and/or a non-coding RNA (ncRNA) targeting library and the like (e.g. a selected set for example based on gene function or pathway).

In certain embodiments, multiple CRISPR-Cas gRNA arrays are provided, which include various combinations of gRNAs to be tested. For example, in some embodiments, two, three, or more gRNAs are included which target different gene transcripts, for testing combinatorial targeting. In other embodiments, dose dependency may be tested, such as by including one, two, three, or more copies of a single gRNA in different CRISPR arrays and assessing the effects. In still other embodiments, positional dependency of gRNAs can be tested, by changing the location of various crRNAs in the CRISPR arrays.

Where a plurality of nucleic acids each comprising different gRNA arrays is used to introduce perturbations in a plurality of cells, it is useful to determine which gRNA array (and thereby the individual gRNAs of the array) is associated with a particular cell. In some embodiments, the methods comprise performing a multiplex CRISPR-based screen using a nucleic acid of the present disclosure comprising a barcode, e.g., as described above. In some cases, the nucleic acid includes a barcode 3′ of the gRNA array. In some instances, the nucleic acid includes a 5′ PCR handle site 3′ of the gRNA array and 5′ of the barcode. In some embodiments, the method comprises isolating RNA from the cell and capturing the RNA comprising the barcode sequence. In some cases, capturing comprises contacting an RNA sample with a nucleotide sequence (e.g., poly(dT)) specific for a reverse transcription handle (e.g., poly(A)) and reverse transcribing the RNA sample (including the barcode sequence). PCR can then be performed, e.g., using a 5′ primer that targets a 5′ PCR handle and a 3′ primer that targets a 3′ PCR handle. In some embodiments, the gRNA array, the nucleotide sequence encoding a 5′ PCR handle site, the nucleotide sequence encoding a barcode are all operably linked to a Pol II promoter (e.g., EF1a) (and therefore are all transcribed as part of the same RNA transcript). In some instances, the capturing nucleotide sequence (the capture sequence) is a poly(dT) sequence (a poly(dT) capture primer), and the reverse transcription handle (the sequence of the transcript that is captured) is a poly(A) tail. In some cases, the method comprises amplifying the barcode sequence using a primer specific for the 5′ PCR handle (and e.g., a 3′ primer specific for a 3′ PCR handle, which can be provided as part of the nucleic acid with the capture sequence, as part of the nucleic acid molecule having the poly(dT)). The method further includes identifying the barcode sequence, e.g., by sequencing. In some embodiments, the method includes sequencing the nucleic acids of the present disclosure to identify the gRNA array and individual gRNAs associated with a particular barcode sequence. Methods of capturing, amplifying, and sequencing nucleic acids are familiar to those of skill in the art (see, e.g., U.S. Pat. Nos. 10,815,525, 10,392,662, and US patent application publication No. US20240124873A1, the disclosures of which are incorporated herein by reference).

The method can further include assessing the modified cell for changes in, e.g., cell viability, cell proliferation, cell apoptosis, cell death, cell phenotype, existence or concentration of a molecule (for example, the target RNA(s)), protein or cell marker expression, or response to a stimulus of a target cell, or a function which may be achieved by the cell culture, tissue, or subject comprising the target cell(s). In some embodiments, the method includes detecting expression of one or more transcripts or gene products. In some embodiments, the method further includes the use of one or more of flow cytometric analysis, cell-hashing, single-cell sequencing analysis, single cell RNA sequencing (scRNA-seq), Perturb-seq, CROP-seq, CRISP-seq, ECCITE-seq., and cellular indexing of transcriptomes and epitopes (CITE-seq).

In some embodiments, the RNA or DNA sequencing occurs by methods that include, without limitation, whole transcriptome analysis, whole genome analysis, barcoded sequencing of whole or targeted regions of the genome, and combinations thereof. In some embodiments, the method comprises detection of cell surface proteins using, e.g. flow cytometry. In certain embodiments, the methods, comprise detection or identification of a CRISPR array as provided herein in combination with profiling additional molecular modalities using methods described in the art, including for example single-cell sequencing analysis (e.g. 10× Genomics Multiome platform), single-cell RNA-sequencing (scRNA-seq) (See, e.g., Haque et al. A practical guide to single-cell RNA-sequencing for biomedical research and clinical applications, Genome Medicine, 9, Article number: 75 (2017); Hwang et al. Single-cell RNA sequencing technologies and bioinformatics pipelines. Exp Mol Med. 2018 Aug. 7; 50(8):96), cell-hashing (See, e.g., Stoeckius et al. Cell Hashing with barcoded antibodies enables multiplexing and doublet detection for single cell genomics. Genome Biol. 2018; 19:224), Perturb-Seq. (See, e.g., Dixit et al. Perturb-seq: Dissecting molecular circuits with scalable single cell RNA profiling of pooled genetic screens. Cell. 2016 Dec. 15; 167(7):1853-1866.e17), CROP-seq (See, e.g., Datlinger et al. Pooled CRISPR screening with single-cell transcriptome readout Nat Methods. 2017 March; 14(3):297-301), CRISP-seq (See, e.g., Jaitin et al. Dissecting Immune Circuits by Linking CRISPR-Pooled Screens with Single-Cell RNA-Seq Cell. 2016 December 15; 167(7):1883-1896.e15), Expanded CRISPR-compatible CITE-seq (ECCITE-seq) (See, e.g., Mimitou et al. Multiplexed detection of proteins, transcriptomes, clonotypes and CRISPR perturbations in single cells. Nat Methods. 2019 May; 16(5):409-41), and cellular indexing of transcriptomes and epitopes-seq (CITE-seq) (See, e.g., Stoeckius et al. Simultaneous epitope and transcriptome measurement in single cells. Nat Methods. 2017 September; 14(9):865-868).

### Kits

Provided are kits/systems for carrying out a subject method. Such kits comprise various combinations of components useful in any of the methods described elsewhere herein.

A kit can further include one or more additional reagents, where such additional reagents can be any convenient reagent. Components of a subject kit can be in separate containers; or can be combined in a single container. In some cases one or more of a kit's components are pharmaceutically formulated for administration to a human.

In addition to above-mentioned components, a subject kit can further include instructions for using the components of the kit to practice the subject methods (e.g., dosing instructions, instructions to administer the component(s) to an individual. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In some embodiments, the instructions are present as an electronic storage data file present on a suitable computer readable storage medium, e.g. CD-ROM, diskette, flash drive, etc. In some embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.
1. A method for identifying CRISPR-Cas guide RNAs (gRNAs) produced in a single cell, comprising:
   capturing a transcript of a CRISPR-Cas guide RNA array using a poly(dT) capture sequence, amplifying a barcode using a 5′ PCR primer that binds to a 5′ PCR handle site, and sequencing the barcode;
      wherein the guide RNA array transcript is expressed from a DNA molecule comprising in 5′ to 3′ order:
         (i) a Pol-II promoter,
         (ii) the CRISPR-Cas guide RNA (gRNA) array,
         (iii) a nucleotide sequence encoding the 5′ PCR handle site, and
         (iv) a nucleotide sequence encoding the barcode;
      wherein (ii), (iii), and (iv) are operably linked to the Pol-II promoter, and
   wherein the start of the 5′ PCR handle site is positioned 5 nucleotides or more 3′ of the 3′-most direct repeat of the CRISPR-Cas gRNA array.
2. The method of 1, wherein the start of the 5′ PCR handle site is positioned 10 nucleotides or more 3′ of the 3′-most direct repeat of the CRISPR-Cas gRNA array.
3. The method of any of 1 to 2, wherein the start of the 5′ PCR handle site is positioned 15 nucleotides or more 3′ of the 3′-most direct repeat of the CRISPR-Cas gRNA array.
4. The method of any of 1 to 3, wherein the cell expresses a CRISPR-Cas effector protein.
5. The method of any of 1 to 3, comprising providing a CRISPR-Cas effector protein or a nucleotide sequence encoding a CRISPR-Cas effector protein to the cell.
6. The method of any of 4 to 5, wherein the CRISPR-Cas effector protein is a Cas12 effector protein or a Cas13 effector protein.
7. The method of any of 1 to 6, further comprising sequencing the DNA molecule.
8. A method for performing multiplex CRISPR screens, comprising:
   contacting a cell population with a plurality of DNA molecules, each DNA molecule comprising in 5′ to 3′ order:
      (i) a Pol-II promoter,
      (ii) a CRISPR-Cas guide RNA (gRNA) array,
      (iii) a nucleotide sequence encoding a 5′ PCR handle site, and
      (iv) a nucleotide sequence encoding a barcode;
      wherein (ii), (iii), and (iv) are operably linked to the Pol-II promoter, and
      wherein the DNA molecule does not comprise a Pol-III promoter.
9. The method of 8, comprising identifying a gRNA array present in individual cells of the cell population, wherein said identifying comprises capturing transcripts expressed from the DNA molecule in a single cell using a poly(dT) capture sequence, amplifying the barcode using a 5′ PCR primer that binds to the 5′ PCR handle site, and sequencing the barcode.
10. The method of any of 8 to 9, comprising sequencing a plurality of transcripts of each cell.
11. The method of any of 8 to 10, comprising sequencing the plurality of DNA molecules.
12. The method of any of 8 to 11, wherein the start of the 5′ PCR handle site is positioned 5 nucleotides or more 3′ of the 3′-most direct repeat of the CRISPR-Cas gRNA array.
13. The method of any of 8 to 12, wherein the start of the 5′ PCR handle site is positioned 10 nucleotides or more 3′ of the 3′-most direct repeat of the CRISPR-Cas gRNA array.
14. The method of any of 8 to 13, wherein the start of the 5′ PCR handle site is positioned 15 nucleotides or more 3′ of the 3′-most direct repeat of the CRISPR-Cas gRNA array.
15. The method of any of 8 to 17, comprising preparing the plurality of DNA molecules.
16. The method of any of 8 to 15, wherein the cells of the cell population express a CRISPR-Cas effector protein.
17. The method of any of 8 to 15, comprising providing a CRISPR-Cas effector protein or a nucleotide sequence encoding a CRISPR-Cas effector protein to the cells of the cell population.
18. The method of any of 16 to 17, wherein the CRISPR-Cas effector protein is a Cas12 effector protein or a Cas13 effector protein.
19. The method of any of 8 to 11, wherein the CRISPR-Cas gRNA array comprises from 1 to 24 gRNAs.
20. The method of any of 8 to 19, wherein the CRISPR-Cas gRNA array comprises gRNAs targeting two or more genes.
21. The method of any of 8 to 20, wherein the CRISPR-Cas gRNA array comprises two or more gRNAs targeting the same gene.
22. The method of any of 8 to 21, wherein the CRISPR-Cas gRNA array comprises 6 gRNAs.
23. The method of any of 8 to 22, wherein the CRISPR-Cas gRNA array comprises gRNAs targeting 6 genes with 1 gRNA per gene, 3 genes with 2 gRNAs per gene, or 2 genes with 3 gRNAs per gene.
24. The method of any of 8 to 23, wherein the barcode of each DNA molecule has a minimum Hamming distance of 4 nucleotides from the barcode of any other DNA molecule.
25. The method of any of 8 to 24, wherein each DNA molecule comprises one or more nucleotide sequences encoding one or more polypeptides 3′ of the Pol-II promoter and 5′ of the gRNA array.
26. The method of any of 8 to 25, wherein each DNA molecule comprises a nucleotide sequence encoding a fluorescent protein.
27. The method of any of 8 to 26, wherein each DNA molecule comprises a nucleotide sequence encoding an antibiotic resistance protein.
28. The method of any of 8 to 27, wherein each DNA molecule comprises a MALAT1 triplex sequence 5′ of the gRNA array.
29. The method of any of 8 to 28, wherein each DNA molecule comprises an RNA stabilization motif 3′ of the gRNA array.
30. The method of any of 8 to 30, wherein the cell population comprises mammalian cells.
31. The method of any of 8 to 30, wherein the cell population comprises human cells.
32. A composition comprising:
   a nucleic acid comprising in 5′ to 3′ order:
      (i) a Pol-II promoter;
      (ii) a CRISPR-Cas guide RNA (gRNA) array;
      (iii) a nucleotide sequence encoding a 5′ PCR handle site; and
      (iv) a nucleotide sequence encoding a barcode;
      wherein (ii), (iii), and (iv) are operably linked to the Pol-II promoter, and
      wherein the start of the 5′ PCR handle site is positioned 5 nucleotides or more 3′ of the 3′-most direct repeat of the CRISPR-Cas gRNA array.
33. The composition of 32, wherein the start of the 5′ PCR handle site is positioned 10 nucleotides or more 3′ of the 3′-most direct repeat of the CRISPR-Cas gRNA array.
34. The composition of any of 32 to 33, wherein the start of the 5′ PCR handle site is positioned 15 nucleotides or more 3′ of the 3′-most direct repeat of the CRISPR-Cas gRNA array.
35. The composition of any of 32 to 34, wherein the nucleic acid does not comprise a Pol-III promoter.
36. The composition of any of 32 to 35, wherein the CRISPR-Cas gRNA array comprises from 1 to 24 gRNAs.
37. The composition of any of 32 to 36, wherein the nucleic acid comprises one or more nucleotide sequences encoding one or more polypeptides 3′ of the Pol-II promoter and 5′ of the gRNA array.
38. The composition of any of 32 to 37, wherein the nucleic acid comprises a nucleotide sequence encoding a fluorescent protein.
39. The composition of any of 32 to 38, wherein the nucleic acid comprises a nucleotide sequence encoding an antibiotic resistance protein.
40. The composition of any of 32 to 39, wherein the nucleic acid comprises a MALAT1 triplex sequence 5′ of the gRNA array.
41. The composition of any of 32 to 40, wherein the nucleic acid comprises an RNA stabilization motif 3′ of the gRNA array.
42. The composition of any of 32 to 41, comprising a plurality of the nucleic acids.
43. The composition of any of 32 to 42, comprising a 5′ PCR primer that hybridizes to the 5′ PCR handle site.
44. The composition of any of 32 to 43, comprising a cell.
45. The composition of 44, wherein the cell expresses a CRISPR-Cas effector protein.
46. The composition of any of 32 to 43, comprising a CRISPR-Cas effector protein or a nucleotide sequence encoding a CRISPR-Cas effector protein.
47. The composition of 45 or 46, wherein the CRISPR-Cas effector protein is a Cas12 effector protein.

### EXPERIMENTAL EXAMPLES

The following examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

General methods in molecular and cellular biochemistry can be found in such standard textbooks as Molecular Cloning: A Laboratory Manual, 3rd Ed. (Sambrook et al., Cold Spring Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like

### Example 1: Pol II-Driven Cas12a Guide Array Expression

Based on observations in the literature that an EF1a promoter can generate uniform guide production across a 20-guide array (Campa et al., 2019), the inventors hypothesized that porting expression of a Cas12a guide array from a Pol III promoter (e.g. mU6, hU6) to a Pol II promoter (e.g. EF1a), may lead to increased levels of guide production, particularly of 3′-positioned guides.

To test this hypothesis, the inventors used a pre-existing lentivirus transfer plasmid containing an mU6 promoter with appropriate guide cloning sites and an EF1a promoter driving expression of puromycin resistance and BFP selection markers. Rather than introducing a second Pol II promoter, the inventors elected to test whether the existing EF1a-PuroR-BFP cassette could be appropriate for expression of a Cas12a guide array.

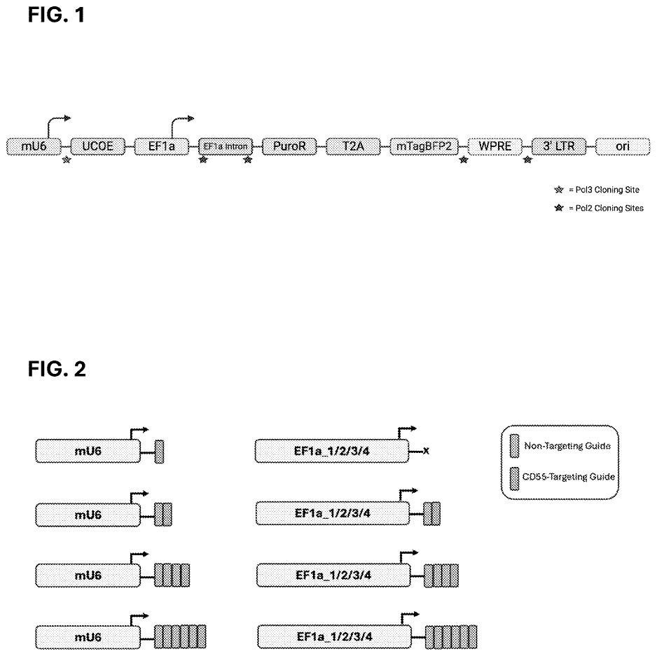

The inventors identified the EF1a promoter intron and the 3′ untranslated region (UTR) as candidate regions for guide array insertion. Cloning into the intron was intended to leverage intronic splicing, with the hope that a guide array embedded within a spliced product could be further processed by the Cas12a enzyme into functional guides. In total, four sites of interest were identified: two within the EF1a intron, and two within the 3′ UTR (FIG. 1). Site-directed mutagenesis (SDM) was used to introduce Blpl and Sphl recognition sequences to each of the four sites of interest. These recognition sequences were selected due to the high fidelity of their corresponding restriction enzymes and the absence of these recognition sequences in the vector backbone. Primers for SDM were designed using NEBaseChanger software and generation of the desired vectors confirmed via whole-plasmid sequencing.

Once the desired vectors were generated, guide arrays were designed and cloned into the target sites. As the literature indicated that the 3′ slot corresponded to the lowest guide production, it was hypothesized that this slot would most sensitively signal changes in guide production arising from changes in array length and/or promoter usage. Thus, a CD55-targeting guide was positioned in the 3′ slot of each array and non-targeting guides in all remaining slots (FIG. 2). 1-, 2-, 4-, and 6-guide arrays were cloned into the mU6 site, and 2-, 4-, and 6-guide arrays into the EF1a_1, EF1a 2, EF1a_3, and EF1a_4 sites. 1-guide arrays were not cloned into the EF1a sites, as the literature indicates that sequences 5′ to the guide array can affect guide processing (Magnusson et al., 2021). The inventors reasoned that it would not be possible to attribute any observed functional differences in 1-guide arrays to the sites themselves rather than the influence of preceding 5′ sequences. The 2-, 4-, and 6-guide arrays do not suffer from this confusion, as the sequence 5′ to the 3′ CD55-targeting guide is held constant across the arrays.

Once the desired plasmids were generated, lentivirus was produced and used to transduce K562 cells expressing Cas12a CRISPRi machinery. Transduction was performed at an MOI of less than 0.3 to achieve primarily single copy integrants. At 7 days post-transduction, a CD55 functional assay was performed. This assay labels cells with a CD55 antibody conjugated to a fluorophore and measures fluorescence intensity via flow cytometry. The proportion of the cell population that was transduced with the guide vector (as measured by BFP) displaying a significant reduction in CD55 expression (as measured by the conjugated fluorophore) is quantified as the % CD55 KD. % KD can vary based on guide sequence and guide abundance; as the same CD55-targeting guide sequence was used across all vectors, the % CD55 KD is interpreted as a proxy for guide abundance.

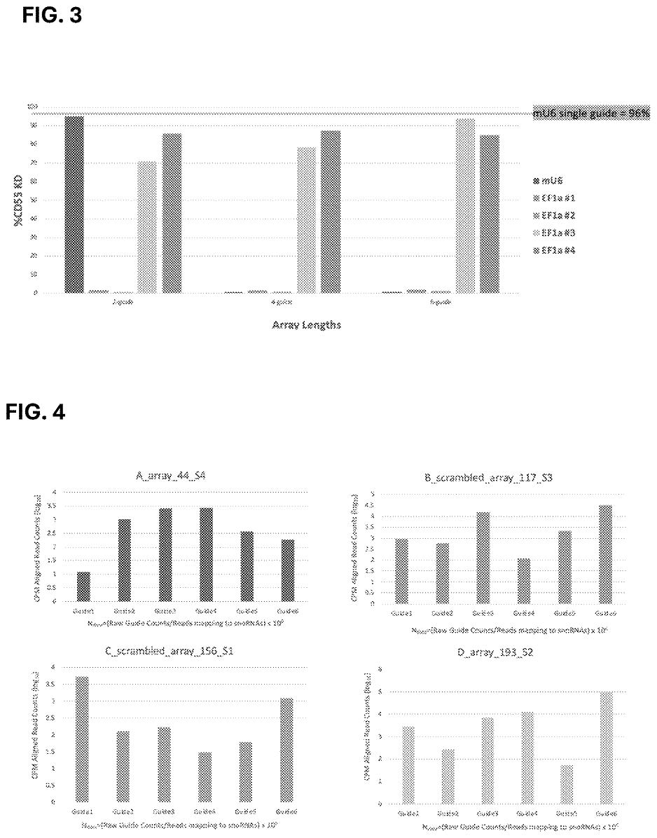

Flow cytometry data (FIG. 3) indicated greater than 95% CD55 KD in cells transduced with mU6-driven 1- and 2-guide arrays, but less than 1% CD55 KD in cells transduced with mU6-driven 4- and 6-guide arrays. In contrast, cells transduced with EF1a_3-driven 2-, 4-, and 6-guide arrays exhibited greater than 70% CD55 KD, while cells transduced with EF1a_4-driven 2-, 4-, and 6-guide arrays exhibited greater than 85% CD55 KD. These data indicate that the EF1a_3 and EF1a_4 sites can produce functional guides out of the 3′ slot of guide arrays of up to 6 guides, while productive 3′ guide expression out of the mU6 site appears limited to arrays of up to 2 (or potentially 3) guides. Cells transduced with EF1a_1-driven and EF1a_2-driven 2-, 4-, and 6-guide arrays exhibited less than 2% CD55 KD, indicating minimal guide production from these sites. These sites were consequently excluded from further study. As the flow cytometry data indicated that the EF1a_4 site was the most productive EF1a site, this was identified as the preferred EF1a cloning site.

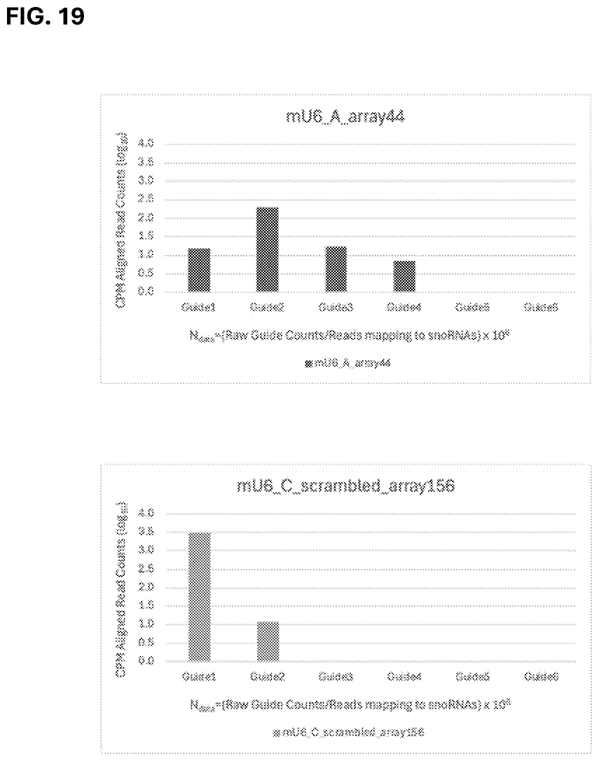

To quantitatively assess individual guide production within a 6-guide array, a small RNA-seq protocol was developed to capture individual guides. four 6-guide arrays, bearing 24 different guide sequences, were cloned into the EF1a_4 site. Lentivirus was produced and used to transduce K562 cells expressing Cas12a CRISPRi machinery. Small RNA-seq libraries were then generated and sequenced on an Illumina NextSeq 550. Reads were aligned to individual guide sequences and normalized by the number of reads in each sample aligning to a snoRNA reference transcriptome (FIG. 4). These data indicated approximately uniform guide abundance across all slots of a 6-guide array and is consistent with published small RNA-seq data (Campa et al., 2019). There do not appear to be clear positional biases influencing guide abundance. It is hypothesized that observed variabilities in guide abundance are due in part to sequence-dependent variability in small RNA capture efficiency. Experiments were also performed to quantify guide production from mU6-driven arrays (FIG. 19). Read counts of individual guides within 6-guide arrays expressed from the mU6 site were determined by small RNA-seq. Reads were aligned to guide sequences and guide read counts normalized by the number of reads within each sample aligning to a snoRNA reference transcriptome. The sequences of mU6-driven guide arrays A and C are identical to corresponding EF1a4-driven guide arrays in FIG. 4. These experiments demonstrate minimal recovery of mU6-driven guides in slots 3, 4, 5, and 6.

### Example 2: Engineering an RNA Secondary Structure to Stabilize Expression of Selection Markers

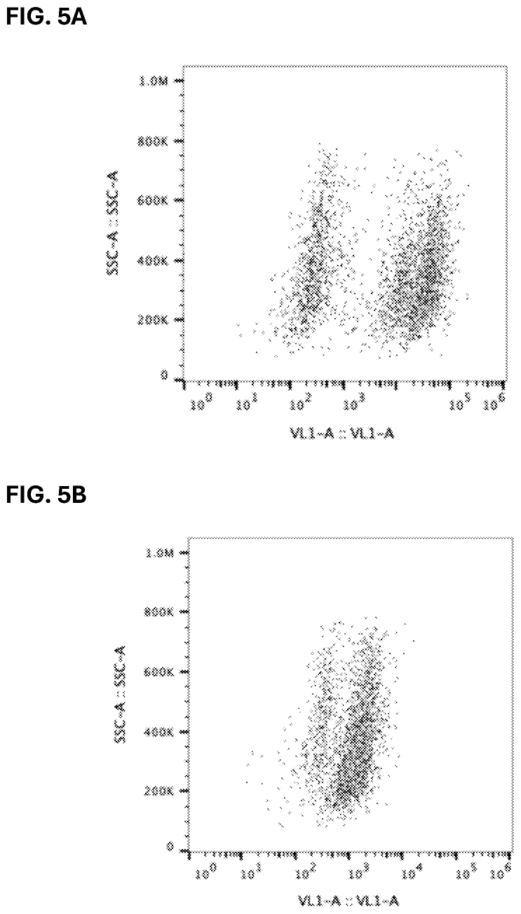

While the EF1a_4 site showed promise in its ability to express functional guides from each slot in a 6-guide array, this site is located in the 3′ UTR of an EF1a cassette expressing puromycin resistance and BFP selection markers. Expression of these selection markers is integral for selecting guide-transduced cells from non-transduced cells. Upon insertion of a 6-guide array into the EF1a_4 site, a greater than 90% decrease was observed in BFP production as measured by flow cytometry (FIG. 5). Reduction in BFP expression was hypothesized to arise from Cas12a-based guide processing, which cleaves the RNA transcript, effectively separating the poly(A) sitail from the protein coding sequences and adversely affecting their stability, nuclear export, and translation.

Despite the observed decrease in BFP expression, there remained sufficient expression to separate guide-transduced cells from non-transduced cells by FACS. However, a greater than 90% decrease in BFP expression (and presumably that of the puromycin resistance gene) poses potential challenges for other experimental modalities, such as antibiotic-resistance and imaging-based selection. To address this issue, an RNA secondary structure was identified from the literature. Termed a triplex structure, the RNA secondary structure forms the 3′ end of the mature MALAT1 non-coding RNA, a non-polyadenylated RNA transcript that is expressed at higher levels than many protein coding genes (Wilusz et al., 2012). The MALAT1 gene encodes both the MALAT1 and mascRNA (MALAT1-associated small cytoplasmic RNA) sequences; RNAse P-mediated cleavage at the junction of these sequences liberates the 3′ end of the MALAT1 RNA, which forms a protective triple helical structure, termed a triplex. Studies in the literature have successfully employed the triplex structure to stabilize a de-polyadenylated Cas12a protein transcript (Campa et al., 2019), indicating its suitability for stabilizing protein coding transcripts. It was thus hypothesized that the triplex structure could potentially help recover expression of the puromycin resistance and BFP selection markers.

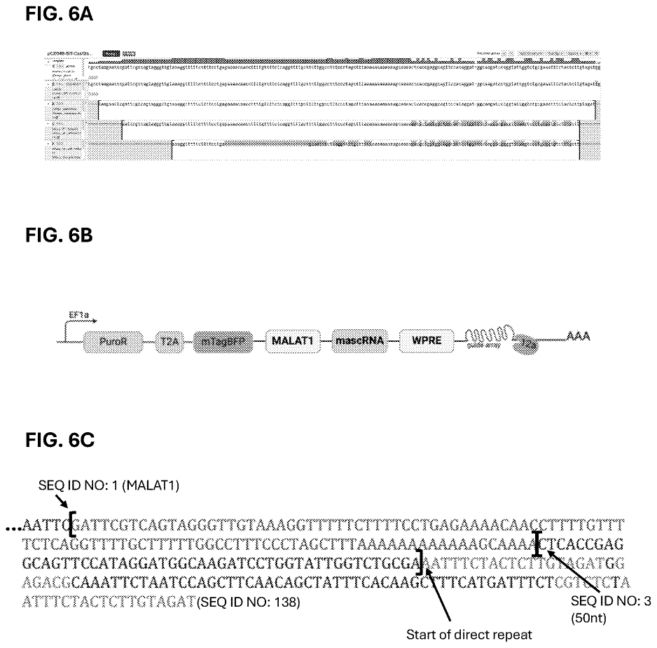

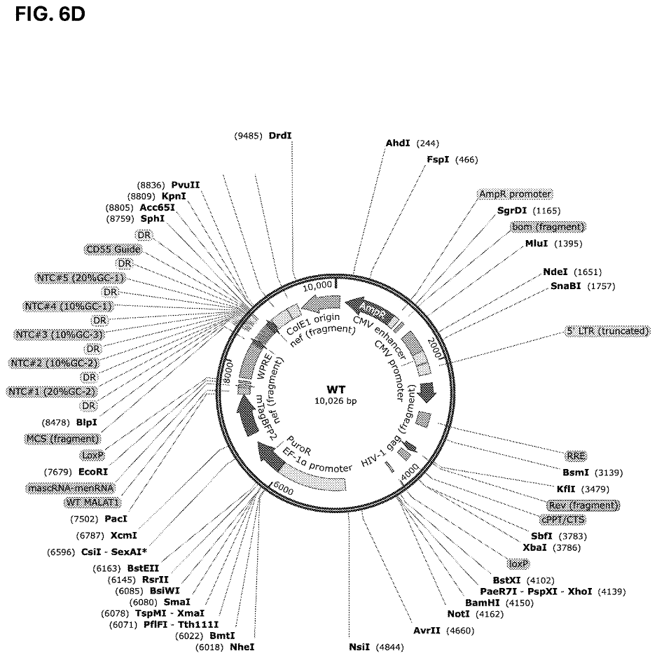

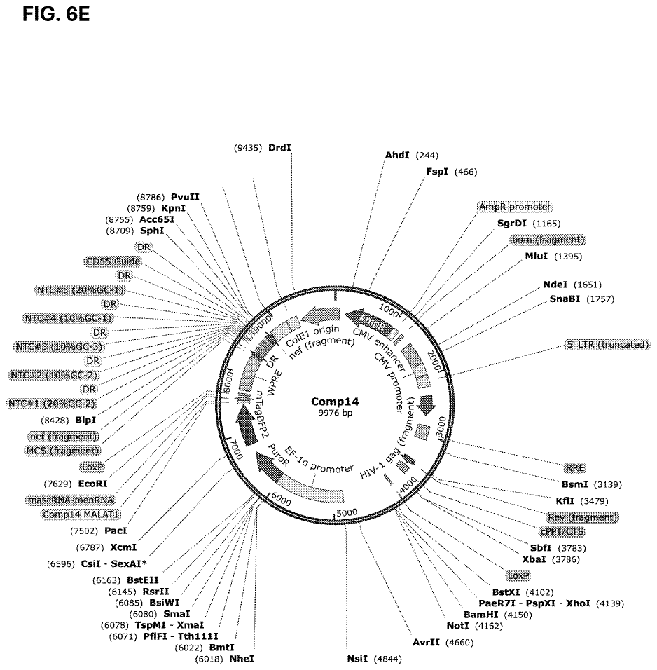

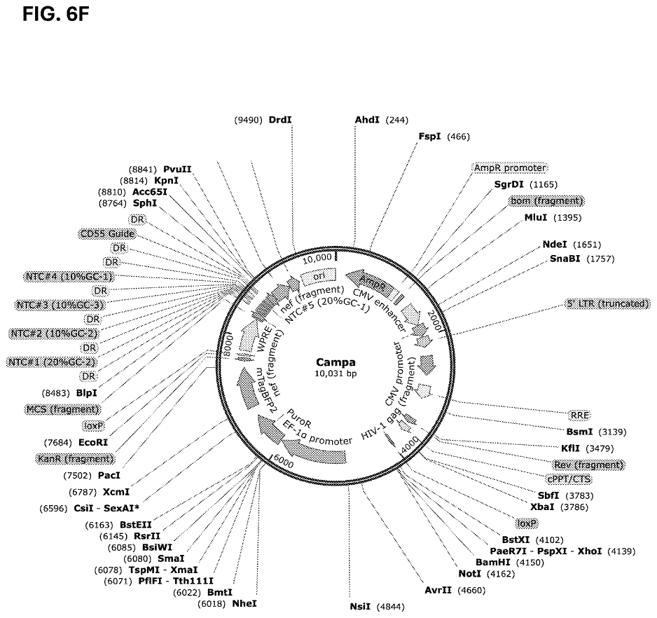

Three different triplex sequences were selected for testing, termed WT (Wilusz et al., 2012), Comp14 (Wilusz et al., 2012), and Campa (Campa et al., 2019). The Campa triplex was identified from an alignment of two vectors (pCE048-SiT-Cas12a, Addgene #128124; pCI152-pLenti-SiT-Cas12a, Addgene #128405) in Campa et al., 2019. The alignment of all three triplex sequences is shown in FIG. 6A. Also see FIG. 6C. The triplex sequence was cloned directly 3′ to the BFP reporter gene, leaving an approximately 800 bp stretch and a WPRE element between the BFP-triplex element and the EF1a_4 site containing a 6-guide array composed of 5 non-targeting guides and a CD55-targeting guide in the 3′ guide slot (FIG. 6B). This guide array was driven under an EF1a promoter along with PuroR and BFP. For each triplex construct, the inventors generated lentivirus and transduced K562 cells expressing Cas12a CRISPRi machinery.

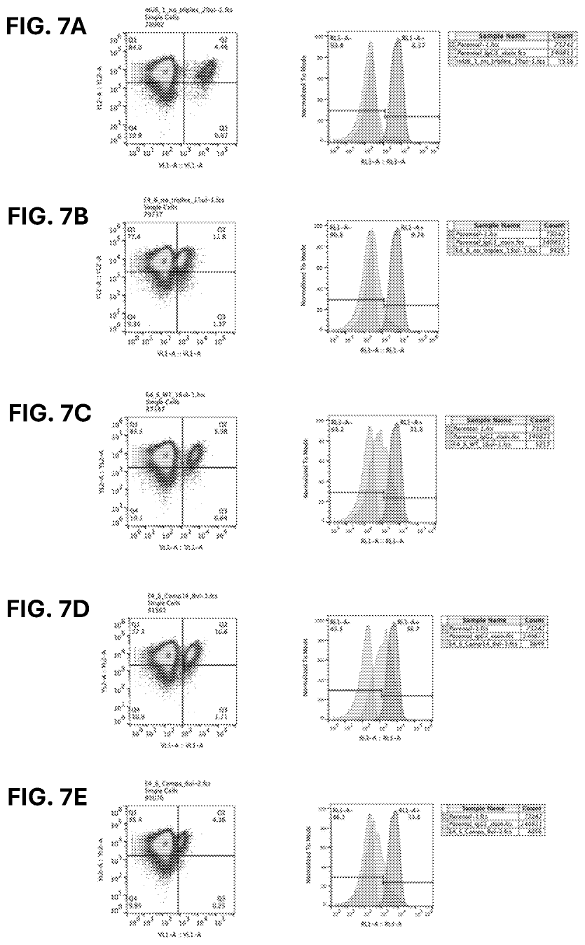

The inventors then measured BFP fluorescence and performed CD55 functional assays (FIG. 7). While the WT and Comp14 triplex constructs led to partial recovery of BFP expression, this coincided with a decrease in % CD55 KD, indicating a decrease in guide production. In contrast, the Campa triplex construct did not lead to recovery of BFP expression, but maintained similar levels of % CD55 KD as the EF1a_4 no triplex 6-guide construct. As such, the WT and Comp14 sequences produced superior transcript stabilization and protein expression when compared to the Campa construct.

Based on Brown et al., 2012, appropriate base pairings to form a triple helical structure are produced by the sequence [ . . . ]AAAAAGCAAAA-3′ at the 3′ terminus of MALAT1. The inventors therefore determined that extraneous bases beyond this sequence may interfere with triple helical structure formation. An excerpt of the sequence used in Campa et al., Nat Methods 16, 887-893 (2019) (see “Supplementary Note 1” of Campa et al.) is shown in FIG. 6C. As indicated in the figure, a 50 nt sequence was present between the MALAT1 sequence (SEQ ID NO: 1 herein) and the first AsCas12a Direct Repeat. This indicates that the Campa sequence as published included extraneous bases at the 3′ terminus of the triplex, potentially interfering with optimal base pairing and formation of a triple helical structure. Thus, the poor performance of the Campa sequence is likely due to the additional 50 nt sequence present at the 3′ terminus.

FIG. 7 demonstrates that while the WT and Comp14 constructs (both retaining a mascRNA sequence) produce stronger BFP signal than the no_triplex and Campa constructs, they result in decreased guide activity as measured by % CD55 knockdown. Although the WT and Comp14 sequences as published (both retaining the MASC sequence) provide a path to produce the desired 3′ terminus sequence and ensuing improved BFP signal, they do not succeed in avoiding losses to guide activity. Therefore, a major challenge is in maintaining production of the desired 3′ terminus sequence without incurring losses in guide activity.

### Example 3: Engineering an RNA Secondary Structure to Stabilize Expression of Guide Arrays

Close inspection of the triplex sequences revealed that both the WT and Comp14 sequences encoded the mascRNA sequence, forming a MALAT1-mascRNA cleavage site. In contrast, the Campa sequence did not encode the mascRNA sequence. It was hypothesized that introduction of the additional cleavage site in the WT and Comp14 triplex constructs may have led to accelerated separation of the 5′ cap from the Cas12a guide array, potentially destabilizing the array and leading to decreased guide production. To address this issue, the inventors hypothesized that removal of the mascRNA sequence and positioning of the guide array directly 3′ to the MALAT1 sequence (removing the 50 nt sequence used in Campa, discussed above) could potentially avoid introduction of an additional cleavage site by using the pre-existing cleavage site of the first guide within the array to liberate the 3′ end of the MALAT1 sequence to form a triplex structure.

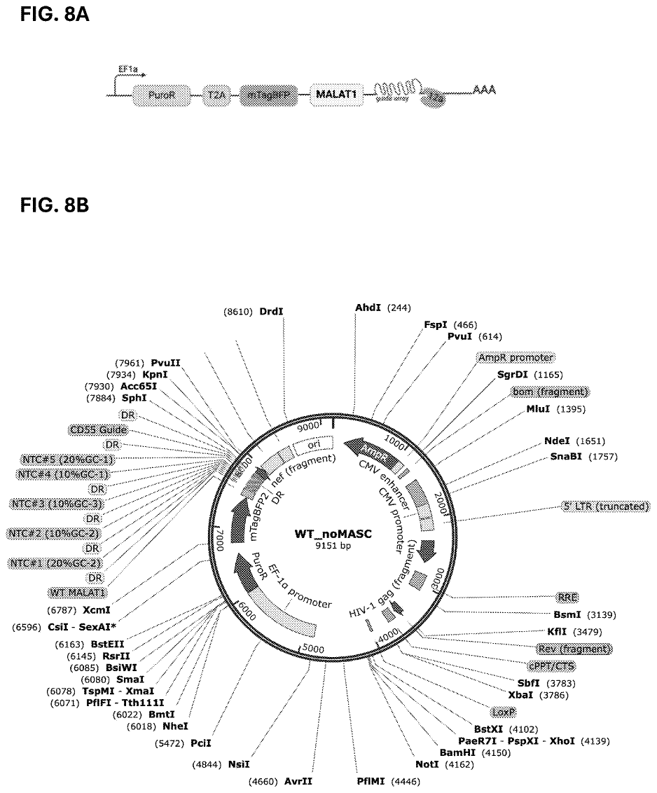

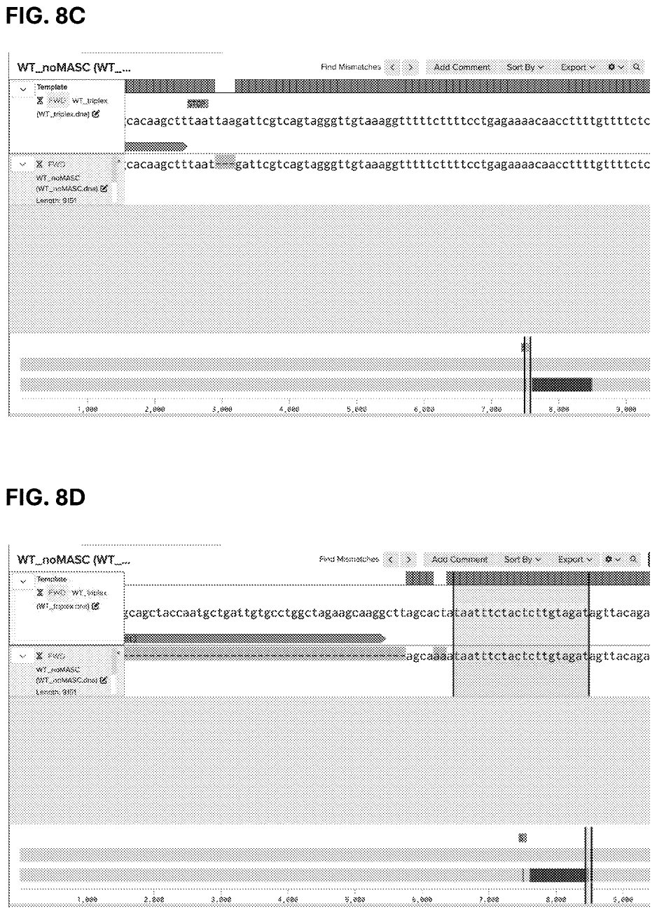

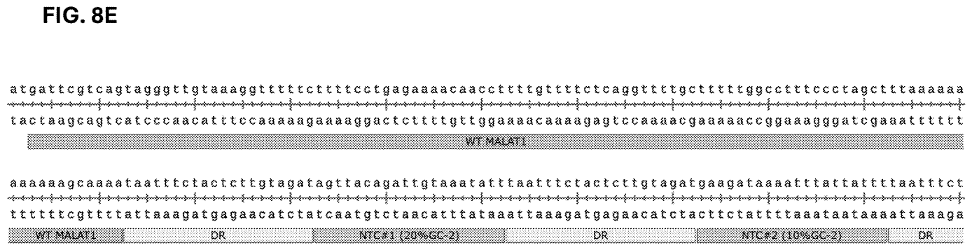

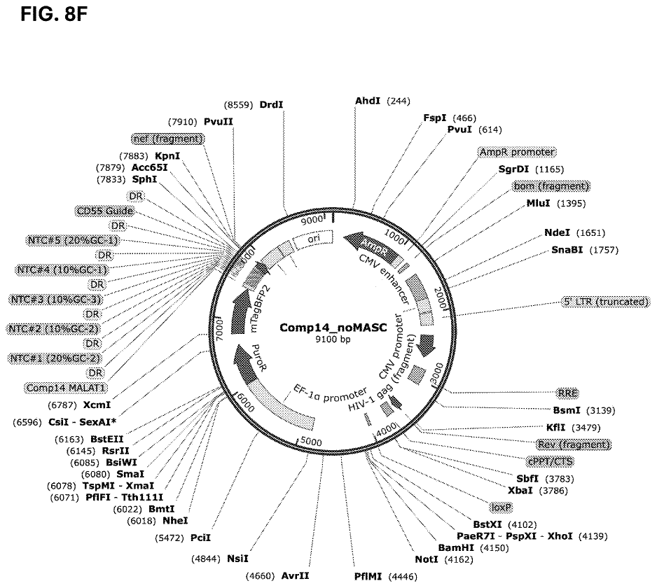

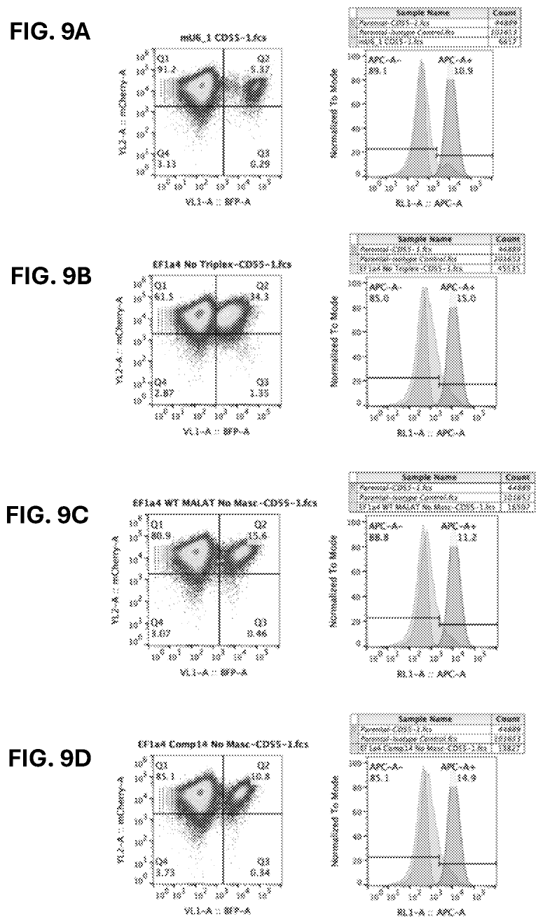

To test this, the inventors focused on the WT and Comp14 triplex sequences and designed and cloned WT_noMASC and Comp14_noMASC constructs. In these constructs, the mascRNA and WPRE elements have been removed, and the EF1a_4 site driving a 6-guide array with a 3′ CD55-targeting guide is positioned directly 3′ to the MALAT1 sequence (FIG. 8). See, e.g., FIG. 8E, which shows an annotated excerpt of the WT_noMASC construct used in the present studies. For each construct, the inventors generated lentivirus and transduced K562 cells expressing Cas12a CRISPRi machinery. The inventors then measured BFP fluorescence and performed CD55 functional assays (FIG. 9). We observed that both the WT_noMASC and Comp14_noMASC constructs led to partial recovery of BFP expression, and, promisingly, did not lead to a decrease in % CD55 KD relative to the EF1a_4 no triplex 6-guide construct. These data indicate that the WT_noMASC and Comp14_noMASC triplex constructs lead to partial recovery of selection marker expression with minimal impact on guide production. Although the BFP signal of WT_noMASC and Comp14_noMASC is not compared directly against the Campa construct, it is noted that the BFP signal of the Campa construct is similar to that of the no_triplex construct (see, e.g., FIG. 7). As the BFP signals from the WT_noMASC and Comp14_noMASC constructs are substantially improved over the no_triplex construct, it can be extrapolated that the WT_noMASC and Comp14_noMASC constructs are likewise improved over the Campa construct.

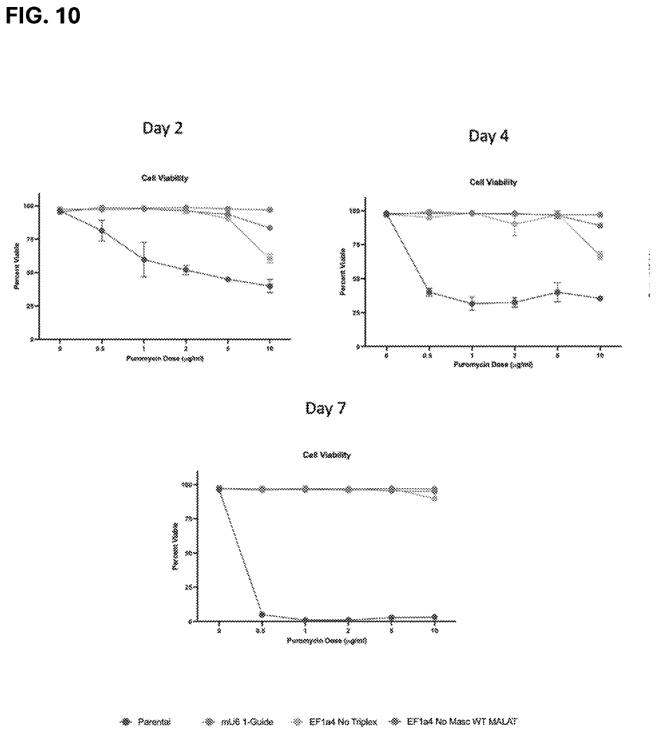

In addition to demonstrating that the WT_noMASC and Comp14_noMASC constructs led to partial recovery of BFP expression, puromycin kill curve assays were also performed that demonstrated the suitability of both constructs for puromycin-based selection and increased puromycin resistance of these constructs relative to the no triplex construct (FIG. 10).

Results from the experiments shown in FIG. 7 and FIG. 9 additionally indicate potential improvements in guide activity in the WT_noMASC construct compared to the no_triplex and/or Campa constructs. As such, it is likely that the WT_noMASC construct produces improved guide activity relative to the no_triplex and/or Campa constructs, and the WT_noMASC sequence can be used to both stabilize protein expression and enhance guide activity.

Therefore, to solve the challenge of maintaining production of the desired 3′ terminus sequence without incurring losses in guide activity, the inventors identified two major insights: a) the importance of producing the desired 3′ terminus sequence, and b) the innovation of co-opting the first cleavage site within the Cas12a array to generate the desired 3′ terminus sequence. Without this insight, production of the 3′ terminus through inclusion of the MASC sequence as originally published in (Wilusz et al., 2012), is not sufficient for maintaining guide activity. The inventors demonstrated that application of these insights is successful in producing improved BFP signal with minimal losses to guide activity as measured by % CD55 knockdown (FIG. 9).

In sum, this invention presents a novel architecture for tandem expression of protein selection markers and Cas12a guide arrays under a single Pol II promoter, attaining sufficient expression of selection markers for fluorescence- and antibiotic-based selection while maintaining suitable levels of guide production.

### Example 4: 5′ PCR Handle Identification for Poly(A)-Based Cas12a Guide Array Capture in Single Cells

Having demonstrated the suitability of the guide vector for driving Cas12a guide arrays of up to 6 guides, additional guide capture capabilities were engineered to allow single-cell Cas12a CRISPR screens. Successful development of this technology facilitates simultaneous Cas12a guide array and mRNA transcriptome capture at the single cell level, providing a high-throughput and information-rich approach to investigating transcriptome-wide effects of multiplexed genetic perturbations.

When developing a strategy for poly(A)-based capture of our Cas12a guide array, we identified appropriate methods to introduce 5′ and 3′ PCR handles. For the 3′ PCR handle, we used a primer containing a poly(dT) homopolymer coupled to a defined constant sequence. For the 5′ PCR handle, a notable advantage of expressing our guide array within an existing EF1a transcript is the existence of unique constant sequences bracketing the guide array. This allows us to bypass use of a TSO and instead use custom primers to generate a 5′ PCR handle. This approach would not be feasible for U6-driven guide arrays, whose 5′ termini typically consist of a Cas12a Direct Repeat (DR) element repeated throughout the array, and whose 3′ termini typically consist of the targeting sequence of the last guide within the array.

Having determined a strategy for introducing a 5′ PCR handle, we identified appropriate priming sites within our transcript sequence. In one approach, the primer is positioned 5′ to the guide array such that the guide array is incorporated in the amplicon. As this approach directly captures and sequences the guide array, it has the benefit of detecting recombinant guide arrays resulting from plasmid or lentiviral recombination events. A significant challenge inherent to this approach lies in the guide processing activity of Cas12a, which catalytically cleaves guide arrays to produce functional single guides. Cleavage at any site along the array results in separation of the 5′ priming site from the poly(A)-tail, precluding PCR amplification.

In a second approach, the primer is positioned 3′ to the guide array, removing the possibility of cleavage-based separation of the priming site from the poly(A) sitail. However, amplification from this site does not directly capture the guide array, a challenge that can be addressed by encoding a set of barcodes 3′ to the priming site. Barcodes can be captured and assigned to single cells, and the corresponding guide arrays determined through deep sequencing of the guide array-barcode plasmid library used in lentivirus generation.

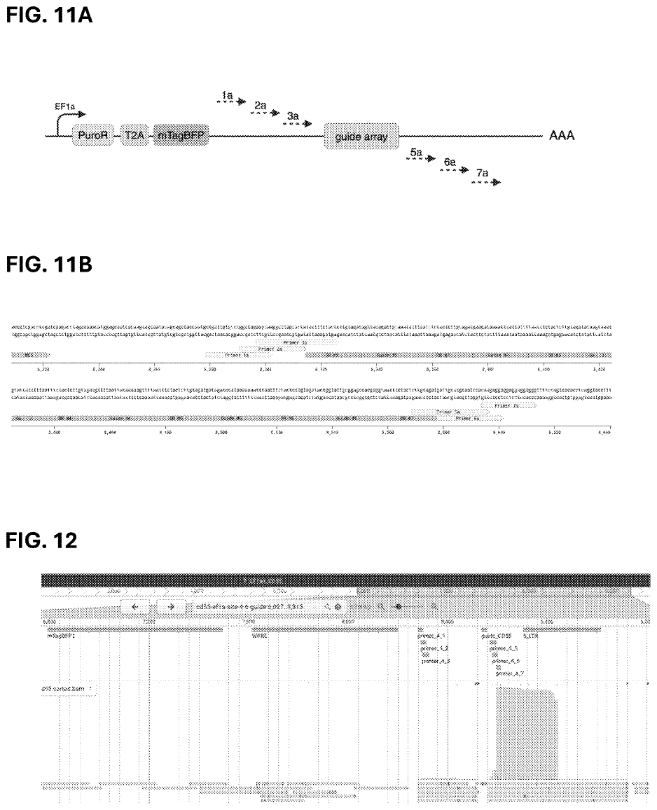

To test these approaches, we designed and tested six different custom primers (1a, 2a, 3a, 5a, 6a, 7a). Primers 1a, 2a, and 3a were positioned 5′ to the guide array, while primers 5a, 6a, and 7a were positioned 3′ to the guide array (FIG. 11A-11B). For this initial set of experiments, we did not introduce barcodes 3′ to the guide array, as they were deemed unnecessary for assessing transcript capture. If transcript capture from primers 5a, 6a, or 7a appeared promising, barcodes would be introduced for subsequent experiments.

The table below shows how far the Start and End base of each primer is, relative to the closest DR.

TABLE 1

| | Primer Start | Primer End |
|---|---|---|
| Primer 1a | −36 nt from DR #1 | −12 nt from DR #1 |
| Primer 2a | −24 nt from DR #1 | 0 nt from DR #1 |
| Primer 3a | −18 nt from DR #1 | +12 nt into DR #1 |
| Primer 5a | −9 nt into DR #7 | +19 nt from DR #7 |
| Primer 6a | 0 nt from DR #7 | +24 nt from DR #7 |
| Primer 7a | +16 nt from DR #7 | +36 nt from DR #7 |

We generated lentivirus from a guide vector containing a 6-guide array with a 3′ CD-55 targeting guide driven out of the EF1a_4 site and transduced a Cas12a CRISPRi-expressing K562 pool. We then ran our cells using the 10× Genomics Chromium Next GEM Single Cell 3′ Reagent Kit v3.1. This kit is designed for mRNA transcriptome capture. We made several modifications to the protocol to account for our use of a custom primer rather than a TSO, along with changes in expected product sizes.

In Step 2.2 (cDNA Amplification) of the protocol, we spiked in 1 μl of a 2.5 μM equimolar mixture of primers 1a, 2a, 3a, 5a, 6a, and 7a to the cDNA amplification master mix. The master mix contains TSO, allowing us to capture mRNA along with the guide array. We followed the 10× protocol for the remaining steps, other than minor modifications to append Illumina adapter sequences and account for changes in expected product sizes. Libraries were sequenced on an Illumina MiSeq. Sequencing reads from the guide array library were aligned against the guide vector map (FIG. 12). Data indicated that, of the 5′ primers, primer 2a produced the highest number of aligned reads, while of the 3′ primers, primer 7a produced the highest number of aligned reads. Primers 2a and 7a were selected for further testing.

### Example 5: Guide Array Barcoding for Poly(A)-Based Capture

We next ran an experiment using a more diverse guide array library to test our ability to assign guide arrays at the single cell level. While use of primer 2a did not require additional engineering of the guide vector, use of primer 7a necessitated introduction of a barcode 3′ to the priming site to facilitate barcode capture and single cell guide array assignment. Associations between guide arrays and barcodes can be either pre-defined or randomly generated. In pre-defined associations, a unique barcode is designated for each guide array. Pre-defined associations can be produced by ordering the guide array and barcode within the same oligonucleotide. The oligonucleotide is cloned into the guide vector as a single element, maintaining the pre-defined guide array-barcode association. In randomly generated associations, guide arrays are not assigned designated barcodes, but instead randomly associate with barcodes within a pool. This can be accomplished through sequential rounds of cloning. In the first round, barcodes are cloned into the vector, generating a pool of barcoded vectors. In the second round, guide arrays are cloned into the pool of barcoded vectors, with each guide array randomly associating with a barcoded vector. Associations are then identified through deep sequencing of the guide array-barcode plasmid library.

We decided to pursue use of random guide array-barcode associations due to length limitations presented by commercial oligo synthesis platforms. Due to cost considerations, when ordering our guide arrays as oligo pools, we limited our oligos to lengths of 300 nt. This allowed us to encode up to six guides per oligo. Use of pre-defined guide array-barcode associations would require addition of both the 7a priming site and the barcode to each oligo, reducing the number of guides encoded within each oligo. In contrast, use of randomly generated guide array-barcode associations would maintain the ability to encode six guides within each oligo, as the guide arrays and barcodes would be ordered and cloned as two separate oligo pools.

To clone the barcodes, we began by ordering 100 nmol of a single oligonucleotide sequence from IDT. This sequence was composed of appropriate restriction digest sites bracketing a 24 bp randomer barcode (N₁N₂N₃. . . N₂₄), representing 424 possible barcode sequences. We undertook two sequential rounds of cloning. We first PCR amplified the barcode oligonucleotide and cloned the product into the guide vector, producing a pool of barcoded guide vectors. We then PCR amplified a 201-element Cas12a guide array oligo pool and cloned the product into our pool of barcoded guide vectors, producing a pool of guide array-barcode vectors. Lentivirus was generated and transduced into Cas12a CRISPRi-expressing K562 cells.

### Example 6: Sequencing and Identification of Guide Arrays Using Poly(A)-Based Capture

We then ran these cells on the 10× platform using a 10× Genomics Chromium Next GEM Single Cell 3′ Reagent Kit v3.1, using our modified protocol. We ran primers 2a and 7a in separate lanes, adding 1 μL of 2.5 μM primer in Step 2.2 (cDNA Amplification). Both the gene expression and guide array (2a) or barcode (7a) libraries were recovered and sequenced on an Illumina 550.

We analyzed sequencing data using a two-step process. First, we used 10× CellRanger software to align sequencing reads to reference transcriptomes and assign transcripts to single cells. We then used the Seurat package to analyze cells by guide array assignments and quantify changes in gene expression. The libraries generated by primers 2a and 7a were analyzed separately.

The 2a gene expression library was analyzed using standard CellRanger inputs. The 2a guide array library was analyzed by appending the 201-element list of guide array sequences to the CellRanger transcriptome reference. CellRanger was used to align sequencing reads against this reference transcriptome and tabulate the number of unique molecular identifiers (UMIs) observed per guide array per cell. This analysis indicated that primer 2a did not capture a sufficient number of guide arrays UMIs per cell to allow single cell guide array assignment. Primer 2a was consequently discarded as a candidate primer for poly(A)-based guide array capture.

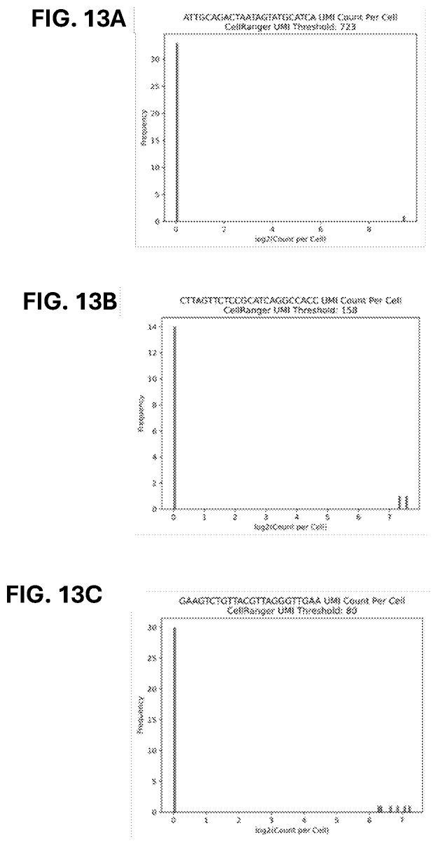

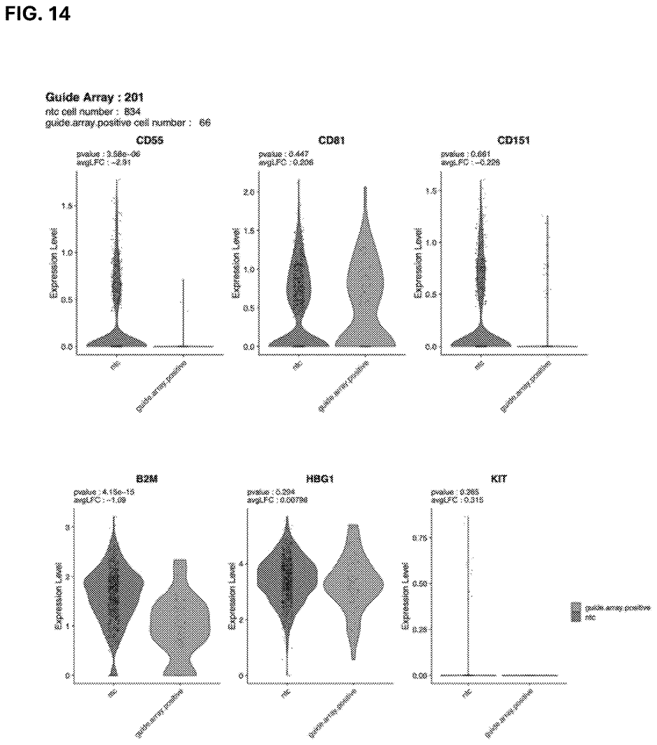

The 7a gene expression library was analyzed using standard CellRanger inputs. The 7a barcode library was analyzed by generating a feature reference file containing all 24 nt barcodes observed within the guide array-barcode plasmid library as determined by deep sequencing of the plasmid library. CellRanger was used to align sequencing reads against this reference file and tabulate the number of UMIs observed per barcode per cell. This analysis indicated that primer 7a captured a sufficient number of barcode UMIs per cell to allow single cell barcode assignment (FIG. 13). Single cell barcode assignments were then converted to single cell guide array assignments on the basis of guide array-barcode assignments determined through deep sequencing of the plasmid library.

While use of primer 7a proved successful in allowing poly(A)-based Cas12a single cell guide array assignment, we encountered a significant challenge stemming from our decision to use a 24 nt randomer as our barcode. Use of a randomer resulted in observation of 200,000+ unique 24 nt barcodes within the plasmid pool, far greater than the typical number of features included in CellRanger feature references files. This resulted in significant computational load and required batch processing. Furthermore, use of a randomer does not permit enforcement of minimum Hamming distances between barcodes. Consequently, PCR and sequencing errors that result in spurious 1 nt deviations are incorrectly identified as valid barcodes. This can decrease the signal-to-noise needed for accurate single cell barcode assignment. To address these issues, we elected to transition to a defined set of 100,000 barcodes, enforcing a minimum Hamming distance of 4 nt between barcodes.

### Example 7: CRISPRi Transcriptional Repression and Guide Array Identification Using Poly(A)-Based Capture

Along with validating our technology for single cell guide array capture and assignment, we sought to understand the ability of our system to affect CRISPRi-based transcriptional repression. We designed and generated a 201-element guide array library targeting highly expressed genes in K562. The guide library consisted of arrays targeting 6 (1 guide per gene), 3 (2 guides per gene), or 2 (3 guides per gene) genes, an array of empirically validated guides (Hsiung et al., 2024), and arrays of non-targeting guides. Lentivirus was produced from this library and transduced into Cas12a CRISPRi-expressing K562 cells. Gene expression and guide array libraries were generated using the 7a primer, following our modified 10× protocol. Sequencing libraries were sequenced on an Illumina NextSeq 550.

Preliminary analysis of the 7a libraries observed 4576 cells assigned single guide arrays and indicated statistically significant transcriptional repression arising from all six slots within an array. We observed that the slots producing downregulation of their targeted genes varied across arrays, and no arrays produced downregulation across all six slots (FIG. 14).

The system described herein can also be used with newly engineered variants of the Cas12a CRISPRi machinery. This initial experiment used a ddAsCas12aUltra (catalytically inactive AsCas12a, Ultra variant). Recently published data (Hsiung et al., 2024) indicate that multiAsCas12a (nickase AsCas12a, Enhanced variant) outperforms catalytically inactive variants, an observation we have corroborated. Transcriptional repression levels may also be increased by transducing the machinery at MOI=5 to produce multi-copy integrants (Hsiung et al., 2024). Finally, the multiAsCas 12a variant benefits from a more permissive PAM than the ddAsCas12aUltra variant, allowing for improved placement of the guide in relation to the TSS.

Taken together, these data demonstrate a novel technology allowing poly(A)-based single cell Cas12a guide array capture and assignment via capture of a 3′ barcode. Initial experiments demonstrate the ability to generate single cell guide array assignments while effecting transcriptional repression from all six slots within a guide array. We expect the functionality of the system to improve with further refinements, including use of improved Cas12a CRISPRi machinery and improvements in guide design. This technology is compatible with any poly(A)-based single cell RNA-seq platform and does not rely on platform-specific capture sequences. This technology should be readily deployable for a wide range of applications benefitting from transcriptomic profiling of multiplexed genetic perturbations.

### Example 8: Inclusion of MALAT1 Triplex in Context of Poly(A)-Based Guide Array Capture

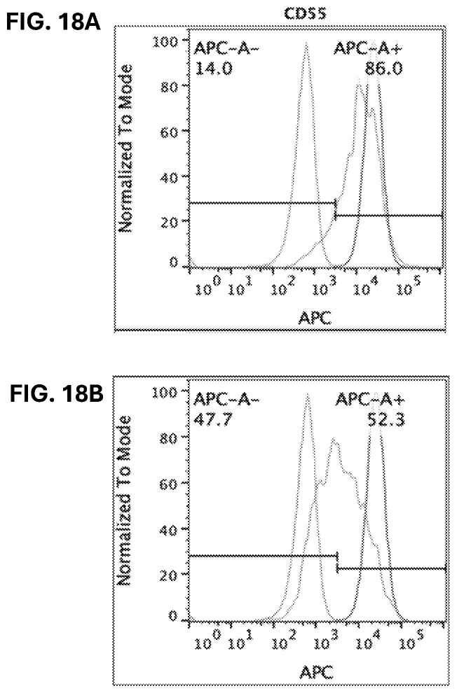

Polypeptide expression and gRNA production can also be increased by including the MALAT1 triplex sequence in vectors used for poly(A)-based capture. This has the benefit of increasing BFP/PuroR expression, which aids in producing a purer population of transduced cells for single cell experiments, where each cell has a non-negligible cost associated. Additionally, our data suggest that inclusion of MALAT1 may increase guide production. In FIG. 9, we observed 88.8% CD55 knockdown with the WT_MALAT1_noMASC construct, compared to 85.0% CD55 knockdown with the “no triplex” construct. These experiments were performed with a cell pool transduced with Cas12a machinery at high enough MOI to generate multiple integrants per cell, essentially boosting the KD capabilities of the cell. In FIG. 18, additional CD55 targeting experiments were performed using the same guide vectors as used in FIGS. 9B and 9C, but using a different cell pool. This cell pool was transduced with Cas12a machinery at an MOI <0.3 to generate primarily single integrant cells to weaken the system and increase the dynamic range so the effects of the triplex on % KD could be better assessed. CD55 KD data from this experiment indicate that the WT_noMASC triplex construct produces greater CD55 KD relative to the no triplex construct.

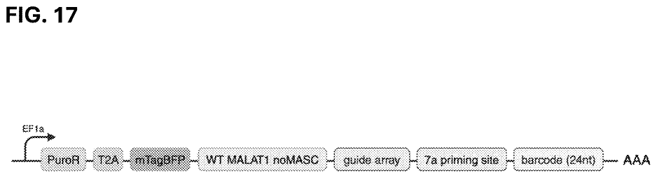

The WT_MALAT1_noMASC architecture can also be used in combination with poly(A)-based guide array capture. FIG. 17 is a diagram of this vector architecture.

### Example 9: CS1-Based Cas12a Guide Array Capture in Single Cells

In Cas9 Perturb-seq, a CS1 is incorporated into each guide constant region, with each guide transcript containing a CS1 element. However, due to commercial oligo synthesis length limitations, this approach is unfavorable for Cas12a guide arrays, as incorporation of one CS1 element per guide would decrease the number of guides that could be encoded within each oligonucleotide. We elected instead to incorporate a single CS1 3′ to the guide array, linking it to a barcode. As with the poly(A)-based approach, this strategy aims to generate single cell barcode assignments and convert these assignments into single cell guide array assignments on the basis of guide array-barcode associations determined through deep sequencing of the plasmid library.

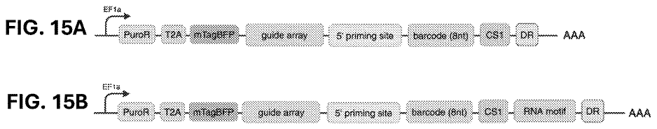

To test our capture strategy, we generated a vector containing a 6-guide array with a 3′ CD55-targeting guide driven out of the EF1a_4 site. We incorporated a CS1 3′ to the guide array. On the 10× platform, CS1 is captured by CS1-complementary oligos coupled to a defined constant sequence. Upon binding of this oligo to CS1, the defined constant sequence acts as a 3′ PCR handle. To generate a 5′ PCR handle, we incorporated a defined constant sequence 5′ to the CS1 and 3′ to the guide array (FIG. 15). This constant sequence can be captured with a custom primer, allowing us to bypass use of a TSO and the associated losses in transcript capture.

In this initial architecture, CS1 is located at the 3′ terminus of the transcript. However, a 3′-terminus CS1 is susceptible to exonucleolytic activity, potentially leading to reduced binding of the complementary oligo and losses in transcript capture. Incorporation of structured secondary RNA motifs 3′ to the CS1 could reduce nucleolytic activity. This hypothesis was supported by experimental data indicating that incorporation of the evopreQ1 RNA motif 3′ to the CS1 resulted in an approximately 10-fold increase in transcript capture.

Based on this observation, we identified seven different structured RNA motifs to test. Of these seven, three were derived from (Wessels et al., 2023), one was derived from (Nelson et al., 2022), and three were designed internally. We cloned these structured RNA motifs into a vector containing a 6-guide array with a 3′ CD-55 targeting guide driven out of the EF1a_4 site and a 5′ custom priming site and 3′ CS1 located 3′ to the guide array. The secondary structures were cloned 3′ to CS1 (FIG. 15). Due to the chemistry of the 10×3′ capture kit, sequences 3′ to CS1 are not captured and RNA motif identities are not captured within the transcript. In order to identify the RNA motif associated with each CS1 capture, we cloned 8 nt barcodes 5′ to CS1, with each RNA motif assigned a unique 8 nt barcode. In total, we designed and generated eight different vector architectures, composed of one architecture with CS1 at the 3′ terminus and seven architectures with a structured RNA motif at the 3′ terminus (FIG. 15).

Lentivirus was generated from each guide vector and transduced into Cas12a CRISPRi-expressing K562 pools, resulting in eight different pools. Cells from each pool were pooled at equal representation and run on a single lane of 10× using the 10× Genomics Chromium Next GEM Single Cell 3′ Reagent Kit v3.1 with Feature Barcoding Technology for CRISPR Screening. This kit is designed from mRNA transcriptome and CS1 capture; we made several modifications to the protocol to account for our use of a custom primer for CS1 capture, along with changes in expected product sizes.

In Step 2.2 (cDNA Amplification) of the protocol, we spiked in 1 μL of 2.5 μM custom primer to the cDNA amplification master mix. The master mix contains TSO, allowing us to capture mRNA along with CS1. We followed the 10× protocol for the remaining steps, other than minor modifications to append Illumina adapter sequences and account for changes in expected product sizes. Both the gene expression and barcode libraries were sequenced on an Illumina NextSeq 550.

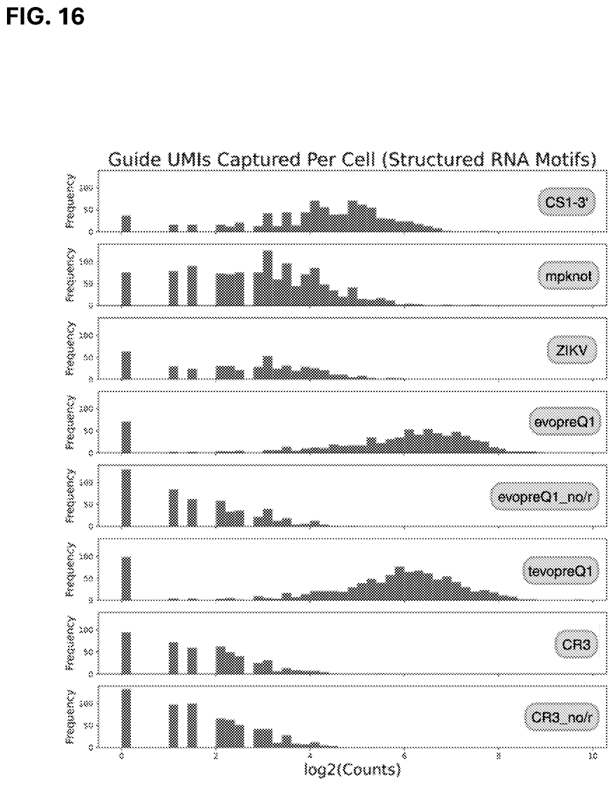

The gene expression library was analyzed using standard CellRanger inputs. The barcode library was analyzed by generating a feature reference file containing the 8 nt barcodes corresponding to the. CellRanger was used to align sequencing reads against this reference file and tabulate the number of UMIs observed per barcode per cell (FIG. 16). Analysis indicated that use of the structured RNA motifs evopreQ1 (Wessels et al., 2023) and tevopreQ1 (Nelson et al., 2022) increased CS1 capture relative to a 3′ CS1, in concordance with published data. Use of these two elements produced sufficiently high numbers of UMIs per cell for single cell barcode assignment. In this proof-of-concept experiment, barcodes were used to test prospective RNA motifs, identify the best-performing motifs, and determine the UMI capture rate for a given motif. Having identified the best-performing RNA motif, application of this capture strategy would no longer employ barcodes to determine associated motifs, but would instead employ barcodes to determine associated guide arrays. The evopreQ1 element would be encoded 3′ to the CS1 and barcode and barcode-guide array pools would be produced through sequential rounds of cloning. CS1-based capture of the barcode would facilitate single cell barcode assignments, which would then be converted into single cell guide array assignments on the basis of guide array-barcode associations determined through deep sequencing of the plasmid library.

The inclusion of RNA motifs without CS1 may further stabilize a guide array for poly(A)-based capture. In the context of the poly(A)-based capture strategy using Primer 7a, RNA motifs can be incorporated 3′ to the guide array and either 5′ or 3′ to the Primer 7a binding site.

### REFERENCES

Brown J. A., Valenstein M. L., Yario T. A., et al. Formation of triple-helical structures by the 3′-end sequences of MALAT1 and MENB noncoding RNAs. PNAS 109(47), 19202-19207 (2012).
Campa, C. C., Weisbach, N. R., Santinha, A. J. et al. Multiplexed genome engineering by Cas12a and CRISPR arrays encoded on single transcripts. Nat Methods 16, 887-893 (2019).
Datlinger, P., Rendeiro, A., Schmidl, C. et al. Pooled CRISPR screening with single-cell transcriptome readout. Nat Methods 14, 297-301 (2017).
Esmaeili Anvar, N., Lin, C., Ma, X. et al. Efficient gene knockout and genetic interaction screening using the in4mer CRISPR/Cas12a multiplex knockout platform. Nat Commun 15, 3577 (2024).
Gilbert, L. A., Horlbeck, M. A., Adamson B., et al. Genome-scale CRISPR-mediated control of gene repression and activation. Cell 159(3), 647-661 (2014).
Horlbeck, M. A., Gilbert, L. A., Villalta, J. E., et al. Compact and highly active next-generation libraries for CRISPR-mediated gene repression and activation. eLife 5:e19760 (2016).
Hsiung, C. CS., Wilson, C. M., Sambold, N. A. et al. Engineered CRISPR-Cas12a for higher-order combinatorial chromatin perturbations. Nat Biotechnol (2024).
Magnusson, J. P., Rios, A. R., Wu, L. et al. Enhanced Cas12a multi-gene regulation using a CRISPR array separator. eLife 10:e66406 (2021).
Nelson J. W., Randolph P. B., Shen S. P., et al. Engineering pegRNAs improve prime editing efficiency. Nat Biotechnol 40(3), 402-410 (2022).
Replogle, J. M., Norman, T. M., Xu, A. et al. Combinatorial single-cell CRISPR screens by direct guide RNA capture and targeted sequencing. Nat Biotechnol 38, 954-961 (2020).
Replogle, J. M., Saunders, R. A., Pogson A. N., et al. Mapping information-rich genotype-phenotype landscapes with genome-scale Perturb-seq. Cell 185(14), 2559-2575.
Wessels, HH., Mendez-Mancilla, A., Hao, Y. et al. Efficient combinatorial targeting of RNA transcripts in single cells with Cas13 RNA Perturb-seq. Nat Methods 20, 86-94 (2023).
Wilusz E. J., JnBaptiste C. K., Lu L. Y. et al. A triple helix stabilizes the 3′ ends of long noncoding RNAs that lack poly(A) tails. Genes Dev 26, 2392-2407 (2012).
Zetsche, B., Heidenreich, M., Mohanraju, P. et al. Multiplex gene editing by CRISPR-Cpf1 using a single crRNA array. Nat Biotechno/35, 31-34 (2017).
10× Chromium Next GEM Single Cell 3′ Reagent Kits v3.1 (Dual Index) User Guide.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method for identifying CRISPR-Cas guide RNAs (gRNAs) produced in a single cell, comprising:
capturing a transcript of a CRISPR-Cas guide RNA array using a poly(dT) capture sequence, amplifying a barcode using a 5′ PCR primer that binds to a 5′ PCR handle site, and sequencing the barcode;
wherein the guide RNA array transcript is expressed from a DNA molecule comprising in 5′ to 3′ order:
(i) a Pol-II promoter,
(ii) the CRISPR-Cas guide RNA (gRNA) array,
(iii) a nucleotide sequence encoding the 5′ PCR handle site, and
(iv) a nucleotide sequence encoding the barcode;
wherein (ii), (iii), and (iv) are operably linked to the Pol-II promoter, and
wherein the start of the 5′ PCR handle site is positioned 5 nucleotides or more 3′ of the 3′-most direct repeat of the CRISPR-Cas gRNA array.

2. The method of claim 1, wherein the start of the 5′ PCR handle site is positioned 10 nucleotides or more 3′ of the 3′-most direct repeat of the CRISPR-Cas gRNA array.

3. (canceled)

4. The method of claim 1, wherein the cell expresses a CRISPR-Cas effector protein.

5-7. (canceled)

8. A method for performing multiplex CRISPR screens, comprising:
contacting a cell population with a plurality of DNA molecules, each DNA molecule comprising in 5′ to 3′ order:
(i) a Pol-II promoter,
(ii) a CRISPR-Cas guide RNA (gRNA) array,
(iii) a nucleotide sequence encoding a 5′ PCR handle site, and
(iv) a nucleotide sequence encoding a barcode;
wherein (ii), (iii), and (iv) are operably linked to the Pol-II promoter, and
wherein the DNA molecule does not comprise a Pol-III promoter.

9. The method of claim 8, comprising identifying a gRNA array present in individual cells of the cell population, wherein said identifying comprises capturing transcripts expressed from the DNA molecule in a single cell using a poly(dT) capture sequence, amplifying the barcode using a 5′ PCR primer that binds to the 5′ PCR handle site, and sequencing the barcode.

10. The method of claim 8, comprising sequencing a plurality of transcripts of each cell.

11. (canceled)

12. The method of claim 8, wherein the start of the 5′ PCR handle site is positioned 5 nucleotides or more 3′ of the 3′-most direct repeat of the CRISPR-Cas gRNA array.

13-15. (canceled)

16. The method of claim 8, wherein the cells of the cell population express a CRISPR-Cas effector protein.

17. (canceled)

19. The method of claim 8, wherein the CRISPR-Cas gRNA array comprises from 1 to 24 gRNAs.

20. The method of claim 8, wherein the CRISPR-Cas gRNA array comprises gRNAs targeting two or more genes.

21-24. (canceled)

25. The method of claim 8, wherein each DNA molecule comprises one or more nucleotide sequences encoding one or more polypeptides 3′ of the Pol-II promoter and 5′ of the gRNA array.

26. The method of claim 8, wherein each DNA molecule comprises a nucleotide sequence encoding a fluorescent protein or an antibiotic resistance protein.

27. (canceled)

28. The method of claim 8, wherein each DNA molecule comprises: (a) a MALAT1 triplex sequence 5′ of the gRNA array, (b) an RNA stabilization motif 3′ of the gRNA array, or both (a) and (b).

29-31. (canceled)

32. A composition comprising:
a nucleic acid comprising in 5′ to 3′ order:
(i) a Pol-II promoter;
(ii) a CRISPR-Cas guide RNA (gRNA) array;
(iii) a nucleotide sequence encoding a 5′ PCR handle site; and
(iv) a nucleotide sequence encoding a barcode;
wherein (ii), (iii), and (iv) are operably linked to the Pol-II promoter, and
wherein the start of the 5′ PCR handle site is positioned 5 nucleotides or more 3′ of the 3′-most direct repeat of the CRISPR-Cas gRNA array.

33. The composition of claim 32, wherein the start of the 5′ PCR handle site is positioned 10 nucleotides or more 3′ of the 3′-most direct repeat of the CRISPR-Cas gRNA array.

34. (canceled)

35. The composition of claim 32, wherein the nucleic acid does not comprise a Pol-III promoter.

36. The composition of claim 32, wherein the CRISPR-Cas gRNA array comprises from 1 to 24 gRNAs.

37. The composition of claim 32, wherein the nucleic acid comprises one or more nucleotide sequences encoding one or more polypeptides 3′ of the Pol-II promoter and 5′ of the gRNA array.

38-39. (canceled)

40. The composition of claim 32, wherein the nucleic acid comprises: (a) a MALAT1 triplex sequence 5′ of the gRNA array, (b) an RNA stabilization motif 3′ of the gRNA array, or (c) both (a) and (b).

41-42. (canceled)

43. The composition of claim 32, comprising a 5′ PCR primer that hybridizes to the 5′ PCR handle site.

44-47. (canceled)
